SEQUENCE LISTING (63 sequences)

SEQ ID NO: 1 - DNA, 2008 bp, Cricetulus griseus
aacagaaacttattttcctgtgtggctaactagaaccagagtacaatgtttccaattctttgagctccgagaagacagaagggagttgaaactctgaaaatgcgggcatggactggttcctggcgttggattatgctcattctttttgcctgggggaccttattgttttatataggtggtcatttggttcgagataatgaccaccctgaccattctagcagagaactctccaagattcttgcaaagctggagcgcttaaaacaacaaaatgaagacttgaggagaatggctgagtctctccgaataccagaaggccctattgatcaggggacagctacaggaagagtccgtgttttagaagaacagcttgttaaggccaaagaacagattgaaaattacaagaaacaagctaggaatgatctgggaaaggatcatgaaatcttaaggaggaggattgaaaatggagctaaagagctctggttttttctacaaagtgaattgaagaaattaaagaaattagaaggaaacgaactccaaagacatgcagatgaaattcttttggatttaggacatcatgaaaggtctatcatgacagatctatactacctcagtcaaacagatggagcaggtgagtggcgggaaaaagaagccaaagatctgacagagctggtccagcggagaataacatatctgcagaatcccaaggactgcagcaaagccagaaagctggtatgtaatatcaacaaaggctgtggctatggatgtcaactccatcatgtggtttactgcttcatgattgcttatggcacccagcgaacactcatcttggaatctcagaattggcgctatgctactggaggatgggagactgtgtttagacctgtaagtgagacatgcacagacaggtctggcctctccactggacactggtcaggtgaagtgaaggacaaaaatgttcaagtggtcgagctccccattgtagacagcctccatcctcgtcctccttacttacccttggctgtaccagaagaccttgcagatcgactcctgagagtccatggtgatcctgcagtgtggtgggtatcccagtttgtcaaatacttgatccgtccacaaccttggctggaaagggaaatagaagaaaccaccaagaagcttggcttcaaacatccagttattggagtccatgtcagacgcactgacaaagtgggaacagaagcagccttccatcccattgaggaatacatggtacacgttgaagaacattttcagcttctcgaacgcagaatgaaagtggataaaaaaagagtgtatctggccactgatgacccttctttgttaaaggaggcaaagacaaagtactccaattatgaatttattagtgataactctatttcttggtcagctggactacacaaccgatacacagaaaattcacttcggggcgtgatcctggatatacactttctctcccaggctgacttccttgtgtgtactttttcatcccaggtctgtagggttgcttatgaaatcatgcaaacactgcatcctgatgcctctgcaaacttccattctttagatgacatctactattttggaggccaaaatgcccacaaccagattgcagtttatcctcaccaacctcgaactaaagaggaaatccccatggaacctggagatatcattggtgtggctggaaaccattggaatggttactctaaaggtgtcaacagaaaactaggaaaaacaggcctgtacccttcctacaaagtccgagagaagatagaaacagtcaaataccctacatatcctgaagctgaaaaatagagatggagtgtaagagattaacaacagaatttagttcagaccatctcagccaagcagaagacccagactaacatatggttcattgacagacatgctccgcaccaagagcaagtgggaaccctcagatgctgcactggtggaacgcctctttgtgaagggctgctgtgccctcaagcccatg

SEQ ID NO: 2 - DNA, 1728 bp, Mus musculus
atgcgggcatggactggttcctggcgttggattatgctcattctttttgcctgggggaccttgttattttatataggtggtcatttggttcgagataatgaccaccctgatcactccagcagagaactctccaagattcttgcaaagcttgaacgcttaaaacagcaaaatgaagacttgaggcgaatggctgagtctctccgaataccagaaggccccattgaccaggggacagctacaggaagagtccgtgttttagaagaacagcttgttaaggccaaagaacagattgaaaattacaagaaacaagctagaaatggtctggggaaggatcatgaaatcttaagaaggaggattgaaaatggagctaaagagctctggttttttctacaaagcgaactgaagaaattaaagcatttagaaggaaatgaactccaaagacatgcagatgaaattcttttggatttaggacaccatgaaaggtctatcatgacagatctatactacctcagtcaaacagatggagcaggggattggcgtgaaaaagaggccaaagatctgacagagctggtccagcggagaataacatatctccagaatcctaaggactgcagcaaagccaggaagctggtgtgtaacatcaataaaggctgtggctatggttgtcaactccatcacgtggtctactgtttcatgattgcttatggcacccagcgaacactcatcttggaatctcagaattggcgctatgctactggtggatgggagactgtgtttagacctgtaagtgagacatgtacagacagatctggcctctccactggacactggtcaggtgaagtaaatgacaaaaacattcaagtggtcgagctccccattgtagacagcctccatcctcggcctccttacttaccactggctgttccagaagaccttgcagaccgactcctaagagtccatggtgaccctgcagtgtggtgggtgtcccagtttgtcaaatacttgattcgtccacaaccttggctggaaaaggaaatagaagaagccaccaagaagcttggcttcaaacatccagttattggagtccatgtcagacgcacagacaaagtgggaacagaagcagccttccaccccatcgaggagtacatggtacacgttgaagaacattttcagcttctcgcacgcagaatgcaagtggataaaaaaagagtatatctggctactgatgatcctactttgttaaaggaggcaaagacaaagtactccaattatgaatttattagtgataactctatttcttggtcagctggactacacaatcggtacacagaaaattcacttcggggtgtgatcctggatatacactttctctcacaggctgactttctagtgtgtactttttcatcccaggtctgtcgggttgcttatgaaatcatgcaaaccctgcatcctgatgcctctgcgaacttccattctttggatgacatctactattttggaggccaaaatgcccacaatcagattgctgtttatcctcacaaacctcgaactgaagaggaaattccaatggaacctggagatatcattggtgtggctggaaaccattgggatggttattctaaaggtatcaacagaaaacttggaaaaacaggcttatatccctcctacaaagtccgagagaagatagaaacagtcaagtatcccacatatcctgaagctgaaaaatag

SEQ ID NO: 3 - DNA, 9196 bp, Cricetulus griseus
tctagaccaggctggtctcgaactcacagagaaccacctgcctctgccacctgagtgctgggattaaaggtgtgcaccaccaccgcccggcgtaaaatcatatttttgaatattgtgataatttacattataattgtaagtaaaaattttcagcctattttgttatacatttttgcgtaaattattcttttttgaaagttttgttgtccataatagtctagggaaacataaagttataatttttgtctatgtatttgcatatatatctatttaatctcctaatgtccaggaaataaatagggtatgtaatagcttcaacatgtggtatgatagaatttttcagtgctatataagttgttacagcaaagtgttattaattcatatgtccatatttcaattttttatgaattattaaattgaatccttaagctgccagaactagaattttattttaatcaggaagccccaaatctgttcattctttctatatatgtggaaaggtaggcctcactaactgattcttcacctgttttagaacatggtccaagaatggagttatgtaaggggaattacaagtgtgagaaaactcctagaaaacaagatgagtcttgtgaccttagtttctttaaaaacacaaaattcttggaatgtgttttcatgttcctcccaggtggataggagtgagtttatttcagattatttattacaactggctgttgttacttgtttctatgtctttatagaaaaacatattttttttgccacatgcagcttgtccttatgattttatacttgtgtgactcttaactctcagagtataaattgtctgatgctatgaataaagttggctattgtatgagacttcagcccacttcaattattggcttcattctctcagatcccaccacctccagagtggtaaacaacttgaaccattaaacagactttagtctttatttgaatgatagatggggatatcagatttataggcacagggttttgagaaagggagaaggtaaacagtagagtttaacaacaacaaaaagtatactttgtaaacgtaaaactatttattaaagtagtagacaagacattaaatattccttgggattagtgctttttgaattttgctttcaaataatagtcagtgagtatacccctcccccattctatattttagcagaaatcagaataaatggtgtttctggtacattcttttgtagagaatttattttctttgggtttttgtgcatttaaagtcaataaaaattaaggttcagtaatagaaaaaaaactctgatttttggaatcccctttcttcagcttttctatttaatctcttaatgataatttaatttgtggccatgtggtcaaagtatatagccttgtatatgtaaatgttttaaccaacctgcctttacagtaactatataattttattctataatatatgacttttcttccatagctttagagttgcccagtcactttaagttacattttcatatatgttctttgtgggaggagataattttatttctaagagaatcctaagcatactgattgagaaatggcaaacaaaacacataattaaagctgataaagaacgaacatttggagtttaaaatacatagccaccctaagggtttaactgttgttagccttcttttggaatttttattagttcatatagaaaaatggattttatcgtgacatttccatatatgtatataatatatttacatcatatccacctgtaattattagtgtttttaaatatatttgaaaaaataatggtctggtttgatccatttgaaccttttgatgtttggtgtggttgccaattggttgatggttatgataacctttgcttctctaaggttcaagtcagtttgagaatatgtcctctaaaaatgacaggttgcaagttaagtagtgagatgacagcgagatggagtgatgagaatttgtagaaatgaattcacttatactgagaacttgttttgcttttagataatgaacatattagcctgaagtacatagccgaattgattaattattcaaagatataatcttttaatccctataaaagaggtattacacaacaattcaagaaagatagaattagacttccagtattggagtgaaccatttgttatcaggtagaaccctaacgtgtgtggttgacttaaagtgtttactttttacctgatactgggtagctaattgtctttcagcctcctggccaaagataccatgaaagtcaacttacgttgtattctatatctcaaacaactcagggtgtttcttactctttccacagcatgtagagcccaggaagcacaggacaagaaagctgcctccttgtatcaccaggaagatctttttgtaagagtcatcacagtataccagagagactaattttgtctgaagcatcatgtgttgaaacaacagaaacttattttcctgtgtggctaactagaaccagagtacaatgtttccaattctttgagctccgagaagacagaagggagttgaaactctgaaaatgcgggcatggactggttcctggcgttggattatgctcattctttttgcctgggggaccttattgttttatataggtggtcatttggttcgagataatgaccaccctgaccattctagcagagaactctccaagattcttgcaaagctggagcgcttaaaacaacaaaatgaagacttgaggagaatggctgagtctctccggtaggtttgaaatactcaaggatttgatgaaatactgtgcttgacctttaggtatagggtctcagtctgctgttgaaaaatataatttctacaaaccgtctttgtaaaattttaagtattgtagcagactttttaaaagtcagtgatacatctatatagtcaatataggtttacatagttgcaatcttattttgcatatgaatcagtatatagaagcagtggcatttatatgcttatgttgcatttacaattatgtttagacgaacacaaactttatgtgatttggattagtgctcattaaatttttttattctatggactacaacagagacataaattttgaaaggcttagttactcttaaattcttatgatgaaaagcaaaaattcattgttaaatagaacagtgcatccggaatgtgggtaattattgccatatttctagtctactaaaaattgtggcataactgttcaaagtcatcagttgtttggaaagccaaagtctgatttaaatggaaaacataaacaatgatatctatttctagatacctttaacttgcagttactgagtttacaagttgtctgacaactttggattctcttacttcatatctaagaatgatcatgtgtacagtgcttactgtcactttaaaaaactgcagggctagacatgcagatatgaagactttgacattagatgtggtaattggcactaccagcaagtggtattaagatacagctgaatatattactttttgaggaacataattcatgaatggaaagtggagcattagagaggatgccttctggctctcccacaccactgtttgcatccattgcatttcacactgcttttagaactcagatgtttcatatggtatattgtgtaactcaccatcagttttatctttaaatgtctatggatgataatgttgtatgttaacacttttacaaaaacaaatgaagccatatcctcggtgtgagttgtgatggtggtaattgtcacaataggattattcagcaaggaactaagtcagggacaagaagtgggcgatactttgttggattaaatcattttactggaagttcatcagggagggttatgaaagttgtggtctttgaactgaaattatatgtgattcattattcttgatttaggccttgctaatagtaactatcatttattgggaatttgtcatatgtgccaatttgtcatgggccagacagcgtgttttactgaatttctagatatctttatgagattctagtactgttttcagccattttacagatgaagaatcttaaaaaatgttaaataatttagtttgcccaagattatacgttaacaaatggtagaaccttctttgaattctggcagtatggctacacagtccgaactcttatcttcctaagctgaaaacagaaaaagcaatgacccagaaaattttatttaaaagtctcaggagagacttcccatcctgagaagatctcttttcccttttataatttaggctcctgaataatcactgaattttctccatgttccatctatagtactgttatttctgttttccttttttcttaccacaaagtatcttgtttttgctgtatgaaagaaaatgtgttattgtaatgtgaaattctctgtccctgcagggtcccacatccgcctcaatcccaaataaacacacagaggctgtattaattatgaaactgttggtcagttggctagggcttcttattggctagctctgtcttaattattaaaccataactactattgtaagtatttccatgtggtcttatcttaccaaggaaagggtccagggacctcttactcctctggcgtgttggcagtgaagaggagagagcgatttcctatttgtctctgcttattttctgattctgctcagctatgtcacttcctgcctggccaatcagccaatcagtgttttattcattagccaataaaagaaacatttacacagaaggacttcccccatcatgttatttgtatgagttcttcagaaaatcatagtatcttttaatactaatttttataaaaaattaattgtattgaaaattatgtgtatatgtgtctgtgtgtcgatttgtgctcataagtagcatggagtgcagaagagggaatcagatctttttttaagggacaaagagtttattcagattacattttaaggtgataatgtatgattgcaaggttatcaacatggcagaaatgtgaagaagctggtcacattacatccagagtcaagagtagagagcaatgaattgatgcatgcattcctgtgctcagctcacttttcctggagctgagctgattgtaagccatctgatgtctttgctgggaactaactcaaaggcaagttcaaaacctgttcttaagtataagccatctctccagtccctcatatggtctcttaagacactttctttatattcttgtacatagaaattgaattcctaacaactgcattcaaattacaaaatagtttttaaaagctgatataataaatgtaaatacaatctagaacatttttataaataagcatattaactcagtaaaaataaatgcatggttattttccttcattagggaagtatgtctccccaggctgttctctagattctactagtaatgctgtttgtacaccatccacaggggttttattttaaagctaagacatgaatgatggacatgcttgttagcatttagacttttttccttactataattgagctagtatttttgtgctcagtttgatatctgttaattcagataaatgtaatagtaggtaatttctttgtgataaaggcatataaattgaagttggaaaacaaaagcctgaaatgacagtttttaagattcagaacaataattttcaaaagcagttacccaactttccaaatacaatctgcagttttcttgatatgtgataaatttagacaaagaaatagcacattttaaaatagctatttactcttgatttttttttcaaatttaggctagttcactagttgtgtgtaaggttatggctgcaaacatctttgactcttggttagggaatccaggatgatttacgtgtttggccaaaatcttgttccattctgggtttcttctctatctaggtagctagcacaagttaaaggtgtggtagtattggaaggctctcaggtatatatttctatattctgtatttttttcctctgtcatatatttgctttctgttttattgatttctactgttagtttgatacttactttcttacactttctttgggatttattttgctgttctaagatttcttagcaagttcatatcactgattttaacagttgcttcttttgtaatatagactgaatgccccttatttgaaatgcttgggatcagaaactcagatttgaacttttcttttttaatatttccatcaagtttaccagctgaatgtcctgatccaagaatatgaaatctgaaatgctttgaaatctgaaacttttagagtgataaagcttccctttaaattaatttgtgttctatattttttgacaatgtcaacctttcattgttatccaatgagtgaacatattttcaatttttttgtttgatctgttatattttgatctgaccatatttataaaattttatttaatttgaatgttgtgctgttacttatctttattattatttttgcttattttctagccaaatgaaattatattctgtattattttagtttgaattttactttgtggcttagtaactgccttttgttggtgaatgcttaagaaaaacgtgtggtctactgatattggttctaatcttatatagcatgttgtttgttaggtagttgattatgctggtcagattgtcttgagtttatgcaaatgtaaaatatttagatgcttgttttgttgtctaagaacaaagtatgcttgctgtctcctatcggttctggtttttccattcatctcttcaagctgttttgtgtgttgaatactaactccgtactatcttgttttctgtgaattaaccccttttcaaaggtttcttttctttttttttttaagggacaacaagtttattcagattacattttaagctgataatgtatgattgcaaggttatcaacatggcagaaatgtgaagaagctaggcacattacatccacatggagtcaagagcagagagcagtgaattaatgcatgcattcctgtggtcagctcacttttcctattcttagatagtctaggatcataaacctggggaatagtgctaccacaatgggcatatccacttacttcagttcatgcaatcaaccaaggcacatccacaggaaaaactgatttagacaacctctcattgagactcttcccagatgattagactgtgtcaagttgacaattaaaactatcacacctgaagccatcactagtaaatataatgaaaatgttgattatcaccataattcatctgtatccctttgttattgtagattttgtgaagttcctattcaagtccctgttccttccttaaaaacctgttttttagttaaataggttttttagtgttcctgtctgtaaatacttttttaaagttagatattattttcaagtatgttctcccagtctttggcttgtattttcatcccttcaatacatatatttttgtaatttattttttttatttaaattagaaacaaagctgcttttacatgtcagtctcagttccctctccctcccctcctcccctgctccccacctaagccccaattccaactcctttcttctccccaggaagggtgaggccctccatgggggaaatcttcaatgtctgtcatatcatttggagcagggcctagaccctccccagtgtgtctaggctgagagagtatccctctatgtggagagggctcccaaagttcatttgtgtactaggggtaaatactgatccactatcagtggccccatagattgtccggacctccaaactgacttcctccttcagggagtctggaacagttctatgctggtttcccagatatcagtctggggtccatgagcaaccccttgttcaggtcagttgtttctgtaggtttccccagcccggtcttgacccctttgctcatcacttctccctctctgcaactggattccagagttcagctcagtgtttagctgtgggtgtctgcatctgcttccatcagctactggatgagggctctaggatggcatataaggtagtcatcagtctcattatcagagaagggcttttaaggtagcctcttgattattgcttagattgttagttggggtcaaccttgtaggtctctggacagtgacagaattctctttaaacctataatggctccctctgtggtggtatcccttttcttgctctcatccgttcctcccctgactagatcttcctgctccctcatgtcctcctctcccctccccttctccccttctctttcttctaactccctctcccctccacccacgatccccattagcttatgagatcttgtccttattttagcaaaacctttttggctataaaattaattaatttaatatgcttatatcaggtttattttggctagtatttgtatgtgtttggttagtgtttttaaccttaattgacatgtatccttatatttagacacagatttaaatatttgaagttttttttttttttttttttaaagatttatttattttttatgtcttctgcctgcatgccagaagagggcaccagatctcattcaaggtggttgtgagccaccatgtggttgctgggaattgaactcaggacctctggaagaacagtcagtgctcttaaccgctgagccatctctccagcccctgaagtgtttcttttaaagaggatagcagtgcatcatttttccctttgaccaatgactcctaccttactgaattgttttagccatttatatgtaatgctgttaccaggtttacattttcttttatcttgctaaatttcttccctgtttgtctcatctcttatttttgtctgttggattatataggcttttatttttctgtttttacagtaagttatatcaaattaaaattattttatggaatgggtgtgttgactacatgtatgtctgtgcaccatgtgctgacctggtcttggccagaagaaggtgtcatattctctgaaactggtattgtggatgttacgaactgccatagggtgctaggaatcaaaccccagctcctctggaaaagcagccactgctctgagccactgagtcctctcttcaagcaggtgatgccaacttttaatggttaccagtggataagagtgcttgtatctctagcacccatgaaaatttatgcattgctatatgggcttgtcacttcagcattgtgtgacagagacaggaggatcccaagagctc

SEQ ID NO: 4 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 5 - protein, 10 aa, Mus musculus
RASSSVSYIH

SEQ ID NO: 6 - protein, 7 aa, Mus musculus
ATSNLAS

SEQ ID NO: 7 - protein, 9 aa, Mus musculus
QQWTSNPPT

SEQ ID NO: 8 - protein, 5 aa, Mus musculus
SYNMH

SEQ ID NO: 9 - protein, 17 aa, Mus musculus
AIYPGNGDTSYNQKFKG

SEQ ID NO: 10 - protein, 12 aa, Mus musculus
STYYGGDWYFNV

SEQ ID NO: 11 - DNA, 384 bp, Mus musculus
atggattttcaggtgcagattatcagcttcctgctaatcagtgcttcagtcataatgtccagaggacaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaa

SEQ ID NO: 12 - protein, 128 aa, Mus musculus
MDFQVQIISFLLISASVIMSRGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIK

SEQ ID NO: 13 - DNA, 420 bp, Mus musculus
atgggttggagcctcatcttgctcttccttgtcgctgttgctacgcgtgtcctgtcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgca

SEQ ID NO: 14 - protein, 140 aa, Mus musculus
MGWSLILLFLVAVATRVLSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSA

SEQ ID NO: 15 - DNA, 91 bp, Artificial Sequence
  Description of Artificial Sequense Synthetic DNA
caggaaacagctatgacgaattcgcctcctcaaaatggattttcaggtgcagattatcagcttcctgctaatcagtgcttcagtcataatg

SEQ ID NO: 16 - DNA, 91 bp, Artificial Sequence
  Description of Artificial Sequense Synthetic DNA
gtgaccttctcccctggagatgcagacaggattgctggagactgggagagaacaatttgtcctctggacattatgactgaagcactgatta

SEQ ID NO: 17 - DNA, 90 bp, Artificial Sequence
  Description of Artificial Sequense Synthetic DNA
ctccaggggagaaggtcacaatgacttgcagggccagctcaagtgtaagttacatccactggttccagcagaagccaggatcctccccca

SEQ ID NO: 18 - DNA, 89 bp, Artificial Sequence
  Description of Artificial Sequense Synthetic DNA
ccagacccactgccactgaagcgaacagggactccagaagccaggttggatgtggcataaatccagggtttgggggaggatcctggctt

SEQ ID NO: 19 - DNA, 91 bp, Artificial Sequence
  Description of Artificial Sequense Synthetic DNA
tcagtggcagtgggtctgggacttcttactctctcaccatcagcagagtggaggctgaagatgctgccacttattactgccagcagtggac

SEQ ID NO: 20 - DNA, 90 bp, Artificial Sequence
  Description of Artificial Sequense Synthetic DNA
gttttcccagtcacgaccgtacgtttgatttccagcttggtcccccctccgaacgtgggtgggttactagtccactgctggcagtaataa

SEQ ID NO: 21 - DNA, 24 bp, Artificial Sequence
  Description of Artificial Sequense Synthetic DNA
gtctgaagcattatgtgttgaagc

SEQ ID NO: 22 - DNA, 23 bp, Artificial Sequence
  Description of Artificial Sequense Synthetic DNA
gtgagtacattcattgtactgtg

SEQ ID NO: 23 - protein, 575 aa, Cricetulus griseus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNDLGKDHEILRRRIENGAKELWFFLQSELKKLKKLEGNELQRHADEILLDLGHHERSIMTDLYYLSQTDGAGEWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVKDKNVQVVELPIVDSLHPRPPYLPLAVPEDLADRLLRVHGDPAVWWVSQFVKYLIRPQPWLEREIEETTKKLGFKHPVIGVHVRRTDKVGTEAAFHPIEEYMVHVEEHFQLLERRMKVDKKRVYLATDDPSLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHQPRTKEEIPMEPGDIIGVAGNHWNGYSKGVNRKLGKTGLYPSYKVREKIETVKYPTYPEAEK

SEQ ID NO: 24 - protein, 575 aa, Mus musculus
MRAWTGSWRWIMLILFAWGTLLFYIGGHLVRDNDHPDHSSRELSKILAKLERLKQQNEDLRRMAESLRIPEGPIDQGTATGRVRVLEEQLVKAKEQIENYKKQARNGLGKDHEILRRRIENGAKELWFFLQSELKKLKHLEGNELQRHADEILLDLGHHERSIMTDLYYLSQTDGAGDWREKEAKDLTELVQRRITYLQNPKDCSKARKLVCNINKGCGYGCQLHHVVYCFMIAYGTQRTLILESQNWRYATGGWETVFRPVSETCTDRSGLSTGHWSGEVNDKNIQVVELPIVDSLHPRPPYLPLAVPEDLADRLLRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPTLLKEAKTKYSNYEFISDNSISWSAGLHNRYTENSLRGVILDIHFLSQADFLVCTFSSQVCRVAYEIMQTLHPDASANFHSLDDIYYFGGQNAHNQIAVYPHKPRTEEEIPMEPGDIIGVAGNHWDGYSKGINRKLGKTGLYPSYKVREKIETVKYPTYPEAEK

SEQ ID NO: 25 - DNA, 99 bp, Artificial Sequence
  Description of Artificial Sequense Synthetic DNA
caggaaacagctatgacgcggccgcgacccctcaccatgggttggagcctcatcttgctcttccttgtcgctgttgctacgcgtgtcctgtcccaggta

SEQ ID NO: 26 - DNA, 98 bp, Artificial Sequence
  Description of Artificial Sequense Synthetic DNA
atgtgtagccagaagccttgcaggacatcttcactgaggccccagccttcaccagctcagccccaggctgctgcagttgtacctgggacaggacacgc

SEQ ID NO: 27 - DNA, 97 bp, Artificial Sequence
  Description of Artificial Sequense Synthetic DNA
caaggcttctggctacacatttaccagttacaatatgcactgggtaaaacagacacctggtcggggcctggaatggattggagctatttatcccgga

SEQ ID NO: 28 - DNA, 99 bp, Artificial Sequence
  Description of Artificial Sequense Synthetic DNA
gtaggctgtgctggaggatttgtctgcagtcaatgtggccttgcctttgaacttctgattgtaggaagtatcaccatttccgggataaatagctccaat

SEQ ID NO: 29 - DNA, 99 bp, Artificial Sequence
  Description of Artificial Sequense Synthetic DNA
aatcctccagcacagcctacatgcagctcagcagcctgacatctgaggactctgcggtctattactgtgcaagatcgacttactacggcggtgactggt

SEQ ID NO: 30 - DNA, 98 bp, Artificial Sequence
  Description of Artificial Sequense Synthetic DNA
gttttcccagtcacgacgggcccttggtggaggctgcagagacggtgaccgtggtccctgcgccccagacattgaagtaccagtcaccgccgtagtaa

SEQ ID NO: 31 - DNA, 25 bp, Artificial Sequence
  Description of Artificial Sequense Synthetic DNA
gagctggtgaagcctggggcctcag

SEQ ID NO: 32 - DNA, 28 bp, Artificial Sequence
  Description of Artificial Sequence Synthetic DNA
atggctcaagctcccgctaagtgcccga

SEQ ID NO: 33 - DNA, 27 bp, Artificial Sequence
  Description of Artificial Sequence Synthetic DNA
tcaagcgtttgggttggtcctcatgag

SEQ ID NO: 34 - DNA, 25 bp, Artificial Sequence
  Description of Artificial Sequence Synthetic DNA
tccggggatggcgagatgggcaagc

SEQ ID NO: 35 - DNA, 24 bp, Artificial Sequence
  Description of Artificial Sequence Synthetic DNA
cttgacatggctctgggctccaag

SEQ ID NO: 36 - DNA, 25 bp, Artificial Sequence
  Description of Artificial Sequence Synthetic DNA
ccacttcagtcggtcggtagtattt

SEQ ID NO: 37 - DNA, 24 bp, Artificial Sequence
  Description of Artificial Sequence Synthetic DNA
cgctcacccgcctgaggcgacatg

SEQ ID NO: 38 - DNA, 32 bp, Artificial Sequence
  Description of Artificial Sequence Synthetic DNA
ggcaggtgctgtcggtgaggtcaccatagtgc

SEQ ID NO: 39 - DNA, 24 bp, Artificial Sequence
  Description of Artificial Sequence Synthetic DNA
ggggccatgccaaggactatgtcg

SEQ ID NO: 40 - DNA, 25 bp, Artificial Sequence
  Description of Artificial Sequence Synthetic DNA
atgtggctgatgttacaaaatgatg

SEQ ID NO: 41 - DNA, 1504 bp, Cricetulus griseus
  CDS (1)..(1119)
atggctcacgctcccgctagctgcccgagctccaggaactctggggacggcgataagggcaagcccaggaaggtggcgctcatcacgggcatcaccggccaggatggctcatacttggcagaattcctgctggagaaaggatacgaggttcatggaattgtacggcgatccagttcatttaatacaggtcgaattgaacatttatataagaatccacaggctcatattgaaggaaacatgaagttgcactatggtgacctcaccgacagcacctgcctagtaaaaatcatcaatgaagtcaaacctacagagatctacaatcttggtgcccagagccatgtcaagatttcctttgacttagcagagtacactgcagatgttgatggagttggcaccttgcggcttctggatgcaattaagacttgtggccttataaattctgtgaagttctaccaggcctcaactagtgaactgtatggaaaagtgcaagaaataccccagaaagagaccacccctttctatccaaggtcgccctatggagcagccaaactttatgcctattggattgtagtgaactttcgagaggcttataatctctttgcggtgaacggcattctcttcaatcatgagagtcctagaagaggagctaattttgttactcgaaaaattagccggtcagtagctaagatttaccttggacaactggaatgtttcagtttgggaaatctggacgccaaacgagactggggccatgccaaggactatgtcgaggctatgtggctgatgttacaaaatgatgaaccagaggactttgtcatagctactggggaagttcatagtgtccgtgaatttgttgagaaatcattcatgcacattggaaagaccattgtgtgggaaggaaagaatgaaaatgaagtgggcagatgtaaagagaccggcaaaattcatgtgactgtggatctgaaatactaccgaccaactgaagtggacttcctgcagggagactgctccaaggcgcagcagaaactgaactggaagccccgcgttgcctttgacgagctggtgagggagatggtgcaagccgatgtggagctcatgagaaccaaccccaacgcctgagcacctctacaaaaaaattcgcgagacatggactatggtgcagagccagccaaccagagtccagccactcctgagaccatcgaccataaaccctcgactgcctgtgtcgtccccacagctaagagctgggccacaggtttgtgggcaccaggacggggacactccagagctaaggccacttcgcttttgtcaaaggctcctctcaatgattttgggaaatcaagaagtttaaaatcacatactcattttacttgaaattatgtcactagacaacttaaatttttgagtcttgagattgtttttctcttttcttattaaatgatctttctatgacccagcaaaaaaaaaaaaaaaaagggatataaaaaaaaaaaaaaaaaaaaaa

SEQ ID NO: 42 - DNA, 17 bp, Artificial Sequence
  Description of Artificial Sequence Synthetic DNA
gccatccagaaggtggt

SEQ ID NO: 43 - DNA, 17 bp, Artificial Sequence
  Description of Artificial Sequence Synthetic DNA
gtcttgtcagggaagat

SEQ ID NO: 44 - DNA, 28 bp, Artificial Sequence
  Description of Artificial Sequence Synthetic DNA
ggcaggagaccaccttgcgagtgcccac

SEQ ID NO: 45 - DNA, 28 bp, Artificial Sequence
  Description of Artificial Sequence Synthetic DNA
gggtgggctgtaccttctggaacagggc

SEQ ID NO: 46 - DNA, 28 bp, Artificial Sequence
  Description of Artificial Sequence Synthetic DNA
ggcgctggcttacccggagaggaatggg

SEQ ID NO: 47 - DNA, 30 bp, Artificial Sequence
  Description of Artificial Sequence Synthetic DNA
ggaatgggtgtttgtctcctccaaagatgc

SEQ ID NO: 48 - DNA, 1316 bp, Cricetulus griseus
gccccgccccctccacctggaccgagagtagctggagaattgtgcaccggaagtagctcttggactggtggaaccctgcgcaggtgcagcaacaatgggtgagccccagggatccaggaggatcctagtgacagggggctctggactggtgggcagagctatccagaaggtggtcgcagatggcgctggcttacccggagaggaatgggtgtttgtctcctccaaagatgcagatctgacggatgcagcacaaacccaagccctgttccagaaggtacagcccacccatgtcatccatcttgctgcaatggtaggaggccttttccggaatatcaaatacaacttggatttctggaggaagaatgtgcacatcaatgacaacgtcctgcactcagctttcgaggtgggcactcgcaaggtggtctcctgcctgtccacctgtatcttccctgacaagaccacctatcctattgatgaaacaatgatccacaatggtccaccccacagcagcaattttgggtactcgtatgccaagaggatgattgacgtgcagaacagggcctacttccagcagcatggctgcaccttcactgctgtcatccctaccaatgtctttggacctcatgacaacttcaacattgaagatggccatgtgctgcctggcctcatccataaggtgcatctggccaagagtaatggttcagccttgactgtttggggtacagggaaaccacggaggcagttcatctactcactggacctagcccggctcttcatctgggtcctgcgggagtacaatgaagttgagcccatcatcctctcagtgggcgaggaagatgaagtctccattaaggaggcagctgaggctgtagtggaggccatggacttctgtggggaagtcacttttgattcaacaaagtcagatgggcagtataagaagacagccagcaatggcaagcttcgggcctacttgcctgatttccgtttcacacccttcaagcaggctgtgaaggagacctgtgcctggttcaccgacaactatgagcaggcccggaagtgaagcatgggacaagcgggtgctcagctggcaatgcccagtcagtaggctgcagtctcatcatttgcttgtcaagaactgaggacagtatccagcaacctgagccacatgctggtctctctgccagggggcttcatgcagccatccagtagggcccatgtttgtccatcctcgggggaaggccagaccaacaccttgtttgtctgcttctgccccaacctcagtgcatccatgctggtcctgctgtcccttgtctaga

SEQ ID NO: 49 - DNA, 23 bp, Artificial Sequence
  Description of Artificial Sequence Synthetic DNA
gatcctgctgggaccaaaattgg

SEQ ID NO: 50 - DNA, 22 bp, Artificial Sequence
  Description of Artificial Sequence Synthetic DNA
cttaacatcccaagggatgctg

SEQ ID NO: 51 - DNA, 1965 bp, Cricetulus griseus
acggggggctcccggaagcggggaccatggcgtctctgcgcgaagcgagcctgcggaagctgcggcgcttttccgagatgagaggcaaacctgtggcaactgggaaattctgggatgtagttgtaataacagcagctgacgaaaagcaggagcttgcttacaagcaacagttgtcggagaagctgaagagaaaggaattgccccttggagttaactaccatgttttcactgatcctcctggaaccaaaattggaaatggaggatcaacactttgttctcttcagtgcctggaaagcctctatggagacaagtggaattccttcacagtcctgttaattcactctggtggctacagtcaacgacttcccaatgcaagcgctttaggaaaaatcttcacggctttaccacttggtgagcccatttatcagatgttggacttaaaactagccatgtacatggatttcccctcacgcatgaagcctggagttttggtcacctgtgcagatgatattgaactatacagcattggggactctgagtccattgcatttgagcagcctggctttactgccctagcccatccatctagtctggctgtaggcaccacacatggagtatttgtattggactctgccggttctttgcaacatggtgacctagagtacaggcaatgccaccgtttcctccataagcccagcattgaaaacatgcaccactttaatgccgtgcatagactaggaagctttggtcaacaggacttgagtgggggtgacaccacctgtcatccattgcactctgagtatgtctacacagatagcctattttacatggatcataaatcagccaaaaagctacttgatttctatgaaagtgtaggcccactgaactgtgaaatagatgcctatggtgactttctgcaggcactgggacctggagcaactgcagagtacaccaagaacacctcacacgtcactaaagaggaatcacacttgttggacatgaggcagaaaatattccacctcctcaagggaacacccctgaatgttgttgtccttaataactccaggttttatcacattggaacaacggaggagtatctgctacatttcacttccaatggttcgttacaggcagagctgggcttgcaatccatagctttcagtgtctttccaaatgtgcctgaagactcccatgagaaaccctgtgtcattcacagcatcctgaattcaggatgctgtgtggcccctggctcagtggtagaatattccagattaggacctgaggtgtccatctcggaaaactgcattatcagcggttctgtcatagaaaaagctgttctgcccccatgttctttcgtgtgctctttaagtgtggagataaatggacacttagaatattcaactatggtgtttggcatggaagacaacttgaagaacagtgttaaaaccatatcagatataaagatgcttcagttctttggagtctgtttcctgacttgtttagatatttggaaccttaaagctatggaagaactattttcaggaagtaagacgcagctgagcctgtggactgctcgaattttccctgtctgttcttctctgagtgagtcggttgcagcatcccttgggatgttaaatgccattcgaaaccattcgccattcagcctgagcaacttcaagctgctgtccatccaggaaatgcttctctgcaaagatgtaggagacatgcttgcttacagggagcaactctttctagaaatcagttcaaagagaaaacagtctgattcggagaaatcttaaatacaatggattttgcctggaaacaggattgcaaatgcaggcatattctatagatctctgggttcttctttctttctcccctctctcctttcctttccctttgatgtaatgacaaaggtaaaaatggccacttctgatggaaaaaaaaaaaaaaaaaaaaaaaaaa

SEQ ID NO: 52 - DNA, 27 bp, Artificial Sequence
  Description of Artificial Sequence Synthetic DNA
caggggtgttcccttgaggaggtggaa

SEQ ID NO: 53 - DNA, 27 bp, Artificial Sequence
  Description of Artificial Sequence Synthetic DNA
cactgagccaggggccacacagcatcc

SEQ ID NO: 54 - DNA, 23 bp, Artificial Sequence
  Description of Artificial Sequence Synthetic DNA
cccctcacgcatgaagcctggag

SEQ ID NO: 55 - DNA, 27 bp, Artificial Sequence
  Description of Artificial Sequence Synthetic DNA
tgccaccgtttcctccataagcccagc

SEQ ID NO: 56 - DNA, 25 bp, Artificial Sequence
  Description of Artificial Sequence Synthetic DNA
atgaagttgcactatggtgacctca

SEQ ID NO: 57 - DNA, 59 bp, Cricetulus griseus
ccgacagcacctgcctagtaaaaatcatcaatgaagtcaaacctacagagatctacaat

SEQ ID NO: 58 - DNA, 25 bp, Artificial Sequence
  Description of Artificial Sequence Synthetic DNA
gacttagcagagtacactgcagatg

SEQ ID NO: 59 - DNA, 25 bp, Artificial Sequence
  Description of Artificial Sequence Synthetic DNA
accttggatagaaaggggtggtctc

SEQ ID NO: 60 - DNA, 125 bp, Cricetulus griseus
ttgatggagttggcaccttgcggcttctggatgcaattaagacttgtggccttataaattctgtgaagttctaccaggcctcaactagtgaactgtatggaaaagtgcaagaaataccccagaaa

SEQ ID NO: 61 - protein, 372 aa, Cricetulus griseus
MAHAPASCPSSRNSGDGDKGKPRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 62 - protein, 321 aa, Cricetulus griseus
MGEPQGSRRILVTGGSGLVGRAIQKVVADGAGLPGEEWVFVSSKDADLTDAAQTQALFQKVQPTHVIHLAAMVGGLFRNIKYNLDFWRKNVHINDNVLHSAFEVGTRKVVSCLSTCIFPDKTTYPIDETMIHNGPPHSSNFGYSYAKRMIDVQNRAYFQQHGCTFTAVIPTNVFGPHDNFNIEDGHVLPGLIHKVHLAKSNGSALTVWGTGKPRRQFIYSLDLARLFIWVLREYNEVEPIILSVGEEDEVSIKEAAEAVVEAMDFCGEVTFDSTKSDGQYKKTASNGKLRAYLPDFRFTPFKQAVKETCAWFTDNYEQARK

SEQ ID NO: 63 - protein, 590 aa, Cricetulus griseus
MASLREASLRKLRRFSEMRGKPVATGKFWDVVVITAADEKQELAYKQQLSEKLKRKELPLGVNYHVFTDPPGTKIGNGGSTLCSLQCLESLYGDKWNSFTVLLIHSGGYSQRLPNASALGKIFTALPLGEPIYQMLDLKLAMYMDFPSRMKPGVLVTCADDIELYSIGDSESIAFEQPGFTALAHPSSLAVGTTHGVFVLDSAGSLQHGDLEYRQCHRFLHKPSIENMHHFNAVHRLGSFGQQDLSGGDTTCHPLHSEYVYTDSLFYMDHKSAKKLLDFYESVGPLNCEIDAYGDFLQALGPGATAEYTKNTSHVTKEESHLLDMRQKIFHLLKGTPLNVVVLNNSRFYHIGTTEEYLLHFTSNGSLQAELGLQSIAFSVFPNVPEDSHEKPCVIHSILNSGCCVAPGSVVEYSRLGPEVSISENCIISGSVIEKAVLPPCSFVCSLSVEINGHLEYSTMVFGMEDNLKNSVKTISDIKMLQFFGVCFLTCLDIWNLKAMEELFSGSKTQLSLWTARIFPVCSSLSESVAASLGMLNAIRNHSPFSLSNFKLLSIQEMLLCKDVGDMLAYREQLFLEISSKRKQSDSEKS

(19) United States
(12) Patent Application Publication
(10) Pub. No.: US 2004/0093621 A1
(43) Pub. Date: May 13, 2004

(54) Antibody composition which specifically binds to CD20

(75) Inventors: Kenya Shitara, Tokyo (JP); Mikiko Sakurada, Tokyo (JP); Kazuhisa Uchida, Tokyo (JP); Toyohide Shinkawa, Tokyo (JP); Mitsuo Satoh, Tokyo (JP); Ryosuke Nakano, Tokyo (JP)

Correspondence Address: NIXON & VANDERHYE, PC, 1100 N GLEBE ROAD, 8TH FLOOR, ARLINGTON, VA, 22201-4714, US

(73) Assignee: KYOWA HAKKO KOGYO CO., LTD, Tokyo (JP)

(21) Appl. No.: 10/327,663

(22) Filed: Dec. 24, 2002

(30) Foreign Application Priority Data
Dec. 25, 2001 (JP) 2001-392753
Apr. 9, 2002 (JP) 2002-106948
Nov. 1, 2002 (JP) 2002-319975

(51) Int. Cl. C12N 5/06

(52) U.S. Cl. 800/6; 435/334

(57) ABSTRACT

The present invention provides an antibody composition which specifically binds to CD20 and comprises an antibody molecule which has complex N-glycoside-linked sugar chains bound to the Fc region; a process for producing the antibody composition; and a medicament comprising the antibody composition.

### BACKGROUND OF THE INVENTION

1. Field of the Invention

The present invention relates to an antibody composition which is useful for treating diseases relating to CD20-positive cells such as B cell lymphoma and the like, a cell for producing the antibody composition, and a process for producing the antibody composition using the cell.

2. Brief Description of the Background Art

Since antibodies have high binding activity, binding specificity and high stability in blood, their applications to diagnosis, prevention and treatment of various human diseases have been attempted [Monoclonal Antibodies Principles and Applications, Wiley-Liss, Inc., Chapter 2.1 (1995)]. Also, production of a humanized antibody such as a human chimeric antibody or a human complementarity determining region (hereinafter referred to as “CDR”)-grafted antibody from an antibody derived from a non-human animal have been attempted by using genetic recombination techniques. The human chimeric antibody is an antibody in which its antibody variable region (hereinafter referred to as “V region”) is an antibody derived from a non-human animal and its constant region (hereinafter referred to as “C region”) is derived from a human antibody. The human CDR-grafted antibody is an antibody in which the CDR of a human antibody is replaced by CDR of an antibody derived from a non-human animal.

It has been found that five classes, namely IBM, IgD, IgG, IgA and IgE, are present in antibodies derived from mammals. Antibodies of a human IgG class are mainly used for diagnosis, prevention and treatment of various human diseases because they have functional characteristics such as long half-life in blood, various effector functions and the like [Monoclonal Antibodies: Principles and Applications, Wiley-Liss, Inc., Chapter 1 (1995)]. The human IgG class antibody is further classified into the following 4 subclasses: IgG1, IgG2, IgG3 and IgG4. A large number of studies have so far been conducted for antibody-dependent cell-mediated cytotoxic activity (hereinafter referred to as “ADCC activity”) and complement-dependent cytotoxic activity (hereinafter referred to as “CDC activity”) as effector functions of the IgG class antibody, and it has been reported that among antibodies of the human IgG class, the IgG1 subclass has the highest ADCC activity and CDC activity [Chemical Immunology, 65, 88 (1997)]. Actually, it has been reported that, although depletion of CD20-positive B cells is detected when an anti-CD20-chimeric antibody of the IgG1 subclass is administered to a monkey, the depletion is not detected when the antibody of the IgG4 class is used. In view of the above, among commercially available antibodies for treatments, most of the anti-tumor humanized antibodies which require high effector functions for the expression of their effects are antibodies of the human IgG1 subclass.

CD20, also called Bp3S, is a polypeptide of about 35 kDa, and was identified as a human B lymphocyte-specific antigen B1 using a monoclonal antibody [J. Immunol., 125, 1678 (1980)]. It is considered that CD20 is a four-transmembrane molecule, functions as a calcium channel, and relates to activation, proliferation and differentiation of B cells [Immunology Today, 15, 450 (1994)]. Expression of CD20 is limited to the stage from pre-B cells to mature B cells, and CD20 is not expressed in undifferentiated cells and plasma cells. Also, since CD20 has such characteristics that CD20 expresses in 90% or more of B cell non-Hodgkin lymphoma and does not internalize into cells even when an antibody is bound thereto, treatment of B cell lymphoma by an anti-CD20 antibody has been attempted for a long time [Blood, 69, 584 (1987)]. However, since a mouse monoclonal antibody was used in the early stage, a human antibody for the mouse antibody (MAMA; Human Anti Mouse Antibody) was induced in the human body and it lacked in the effector function, so that its therapeutic effect was limited. Accordingly, an attempt was made to prepare a chimeric antibody of a mouse antibody with a human IgG1 subclass using genetic recombination techniques [J. Immunol., 139, 3521 (1987), WO 88/04936]. In addition, it has been confirmed by tests using monkeys that a chimeric antibody IDEC-C2B8, a human IgG1 subclass prepared using a mouse monoclonal antibody 2B8 has an activity to deplete CD20-positive cells even in the living body [Blood, 83, 435 (1994), WO 94/11026], and this antibody was put on the market in November, 1997, in the United States as Rituxan™ (manufactured by IDEC/Genentech, also called Rituximab, and hereinafter referred to as “Rituxan™”) via clinical tests.

The phase m study of Rituxan™ in the United States was carried out by administration to 166 cases of relapsed low grade and follicular lymphomas at a dose of 375 mg/m²/week for 4 weeks, and the efficacy was 48% (complete remission; 6%, partial remission: 42%) [J. Clin. Oncol., 16, 2825 (1998)]. As the action mechanism of Rituxan™, activity to induce apoptosis in cells by crosslinking CD20 in addition to its ADCC activity and CDC activity are considered [Current Opinion in Immunology, 11, 541 (1999)]. Regarding the CDC activity, since the sensitivity varies depending on the target B lymphoma cell, discussions have been made on a possibility of increasing therapeutic effect of Rituxan™ by inhibiting the function of complement inhibitory molecules CD55 and CD59 considered to relate its control [Current Opinion in Immunology, 1, 541 (1999)]. However, it has been also reported that the expression of these inhibitory molecules in tumor cells of patients and in vitro sensitivity of the CDC activity are not always correlative with clinical results [Blood, 9, 1352 (2001)]. In addition, it has been shown by an examination using a model mouse transplanted with a human B lymphoma cell line Raji cell that the ADCC activity via an antibody receptor (hereinafter, the antibody receptor is called FcγR) is important for the antitumor effect [Nature Medicine, 6, 443 (2000)].

Combined use of Rituxan™ and chemotherapy (CHOP; Cyclophosphamide, Doxorubicin, Vincristine, Prednisone) has been examined, and it has been reported that the efficacy in the phase II study was 95% (complete remission; 55%, partial remission; 45%) in 40 cases of low-grade and follicular lymphomas, but with side effects caused by CHOP. [J. Clin. Oncol., 17, 268 (1999)]. In addition, radioisotope-labeled antibodies such as Zevalin (manufactured by IDEC) and Bexxar (manufactured by Corixa) have been developed as other anti-CD20 antibodies for treatments, but since both of them are mouse antibodies and a radioactive isotope is used therein, there is a possibility of causing side effects due to their strong toxicity.

Expression of ADCC activity and CDC activity of the human IgG1 subclass antibodies requires binding of the Fc region of the antibody to an antibody receptor existing on the surface of an effector cell, such as a killer cell, a natural killer cell, an activated macrophage or the like and various complement components. Regarding the binding, it has been suggested that several amino acid residues in the hinge region and the second domain of C region (hereinafter referred to as “Cy2 domain”) of the antibody are important [Eur. J. Immunol., 23, 1098 (1993); Immunology, 86, 319 (1995); Chemical Immunology, 65, 88 (1997); Chemical Immunology, 65, 88 (1997)]. Regarding Rituxan™, as a result of the study using the antibody in which an amino acid of the Cγ2 domain was substituted, amino acids which are mainly important for CDC activity have been identified [J. Immunol., 164, 4178 (2000); J. Immunol., 166, 2571 (2001)].

Furthermore, importance of a sugar chain bound to the Cγ2 domain is suggested [J. Immunology, 65, 88 (1997)]. Regarding the sugar chain, Boyd et al. have examined effects of a sugar chain on the ADCC activity and CDC activity by treating a human CDR-grafted antibody CAMPATH-1H (human IgG1 subclass) produced by a Chinese hamster ovary cell (hereinafter referred to as “CHO cell”) or a mouse myeloma NSO cell (hereinafter referred to as “NSO cell”) with various sugar hydrolyzing enzymes, and reported that elimination of the non-reducing end sialic acid did not have influence upon both activities, but the CDC activity alone was affected by further elimination of galactose residue and about 50% of the activity was decreased, and that complete elimination of the sugar chain caused disappearance of both activities [Molecular Immunol., 32, 1311 (1995)]. Also, Lifely et al. have analyzed the sugar chain bound to a human CDR-grafted antibody CAMPATH-1H (human IgG1 subclass) which was produced by CHO cell, NSO cell or rat myeloma YO cell, measured its ADCC activity, and reported that the CAMPATH-1H derived from YO cell showed the highest ADCC activity, suggesting that N-acetylglucosamine (hereinafter referred also to as “GlcNAc”) at the bisecting position is important for the activity [Glycobiology, 5, 813 (1995); WO 99/54342]. These reports indicate that the structure of the sugar chain plays an important role in the effector functions of human antibodies of IgG1 subclass and that it is possible to prepare an antibody having higher effector function by changing the structure of the sugar chain. However, actually, structures of sugar chains are various and complex, and it cannot be said that an actual important structure for the effector function was identified.

As an example of the modification of the sugar chain structure of a product by introducing a gene of an enzyme relating to the modification of sugar chains into a host cell, it has been reported that a protein in which sialic acid is added in a large number to the non-reducing end of a sugar chain can be produced by introducing rat β-galactoside-α2,6-sialyltransferase into CHO cell [J. Bol. Chem., 261, 13848 (1989)].

Also, expression of an H antigen in which fucose (hereinafter referred also to as “Fuc”) is bound to the non-reducing end of a sugar chain (Fucα1-2Galβ1-) has been confirmed by introducing human O-galactoside-2-α-fucosyltransferase into a mouse L cell [Science, 252, 668 (1991)]. Furthermore, based on the knowledge that binding of the bisecting N-acetylglucosamine of N-glycoside-linked sugar chains is important for the ADCC activity of antibodies, Umana et al. have prepared a β-1,4-N-acetylglucosamine transferase m (GnTIII)-expressing CHO cell and compared with the parent strain. No expression of GnTIII was observed in the parent CHO cell [J Biol. Chem., 5, 13370 (1984)], and it has been confirmed that the antibody expressed using the prepared GnTIII-expressing CHO cell has higher ADCC activity than the antibody expressed in the parent cell [Nature Biotechnol., 17, 176 (1999); WO 99/54342]. In this case, Umana et al. have also prepared a β-1,4-N-acetylglucosamine transferase V (GnTV) gene-introduced CHO cell and reported that over-expression of GnTIII or GnTV shows toxicity upon CHO cell. Regarding Rituxan™, it has been reported that the antibody prepared using the GnTIII-introduced CHO cell shows higher ADCC activity than the antibody expressed in the parent cell, and difference in the activity is approximately 10 to 20 times [Biolechnol. Bioeng., 74, 288 (2001)].

Since the effector function-enhanced anti-CD20 antibody shows increased therapeutic effects, alleviation of patient's burden can be expected by its reduced dose. In addition, other effects such as reduction of side effects can also be expected, because combined use with chemotherapy, a radioactive isotope or the like becomes unnecessary.

### SUMMARY OF THE INVENTION

An object of the present invention is to provide an anti-CD20 antibody-producing cell in which an effector function is enhanced, an anti-CD-20 antibody composition in which an effector function is enhanced, a process for producing the antibody composition, a medicament comprising the antibody composition, and the like.

The present invention relates to the following (1) to (48).

(1) A cell which produces an antibody composition comprising an antibody molecule which specifically binds to CD20 and has complex N-glycoside-linked sugar chains bound to the Fc region, wherein among the total complex N-glycoside-linked sugar chains bound to the Ec region in the composition, the ratio of a sugar chain in which fucose is not bound to N-acetylglucosamine in the reducing end in the sugar chain is 20% or more.

(2) The cell according to (1), wherein the sugar chain to which fucose is not bound is a complex M-glycoside-linked sugar chain in which 1-position of fucose is not bound to 6-position of N-acetylglucosamine in the reducing end through α-bond.

(3) The cell according to (1) or (2), wherein the activity of an enzyme relating to the synthesis of an intracellular sugar nucleotide, GDP-fucose, and/or the activity of an enzyme relating to the modification of a sugar chain in which 1-position of fucose is bound to 6-position of N-acetylglucosamine in the reducing end through α-bond in the complex N-glycoside-linked sugar chain is decreased or deleted.

(4) The cell according to (3), wherein the enzyme relating to the synthesis of an intracellular sugar nucleotide, GDP-fucose is an enzyme selected from the group consisting of the following (a), (b) and (c):

(a) GMD (GDP-mannose 4,6-dehydratase);

(b) Fx (GDP-keto-6-deoxymannose 3,5-epimerase, 4-reductase);

(c) GFPP (GDP-beta-L-fucose pyrophosphorylase).

(5) The cell according to (4), wherein the GMD is a protein encoded by a DNA of the following (a) or (b):

(a) a DNA comprising the nucleotide sequence represented by SEQ ID NO:41;

(b) a DNA which hybridizes with the DNA consisting of the nucleotide sequence represented by SEQ ID NO:41 under stringent conditions and encodes a protein having GMD activity.

(6) The cell according to (4), wherein the GMD is a protein selected from the group consisting of the following (a), (b) and (c):

(a) a protein comprising the amino acid sequence represented by SEQ ID NO:61;

(b) a protein which consists of an amino acid sequence in which at least one amino acid is deleted, substituted, inserted and/or added in the amino acid sequence represented by SEQ ID NO:61 and has GMD activity;

(c) a protein which consists of an amino acid sequence having a homology of at least 80% with the amino acid sequence represented by SEQ ID NO:61 and has GMD activity.

(7) The cell according to (4), wherein the Fxis a protein encoded by a DNA of the following (a) or (b):

(a) a DNA comprising the nucleotide sequence represented by SEQ ID NO:48;

(b) a DNA which hybridizes with the DNA consisting of the nucleotide sequence represented by SEQ ID NO:48 under stringent conditions and encodes a protein having Fx activity.

(8) The cell according to (4), wherein the Fx is a protein selected from the group consisting of the following (a), (b) and (c):

(a) a protein comprising the amino acid sequence represented by SEQ ID NO:62;

(b) a protein which consists of an amino acid sequence in which at least one amino acid is deleted, substituted, inserted and/or added in the amino acid sequence represented by SEQ 1) NO:62 and has Fx activity,

(c) a protein which consists of an amino acid sequence having a homology of at least 80% with the amino acid sequence represented by SEQ ID NO:62 and has Fx activity.

(9) The cell according to (4), wherein the GFPP is a protein encoded by a DNA of the following (a) or (b):

(a) a DNA comprising the nucleotide sequence represented by SEQ ID NO:51;

(b) a DNA which hybridizes with the DNA consisting of the nucleotide sequence represented by SEQ ID NO:51 under stringent conditions and encodes a protein having GFPP activity.

(10) The cell according to (4), wherein the GFPP is a protein selected from the group consisting of the following (a), (b) and (c):

(a) a protein comprising the amino acid sequence represented by SEQ ID NO:63;

(b) a protein which consists of an amino acid sequence in which at least one amino acid is deleted, substituted, inserted and/or added in the amino acid sequence represented by SEQ ID NO:63 and has GFPP activity;

(c) a protein which consists of an amino acid sequence having a homology of at least 80% with the amino acid sequence represented by SEQ ID NO:63 and has GFPP activity.

(11) The cell according to (3), wherein the enzyme relating to the modification of a sugar chain in which 1-position of fucose is bound to 6-position of the N-acetylglucosamine in the reducing end through α-bond in the complex N-glycoside-linked sugar chain is α1,6-fucosyltransferase.

(12) The cell according to (11), wherein the c1,6-fucosyltransferase is a protein encoded by a DNA of the following (a), (b), (c) and (d):

(a) a DNA comprising the nucleotide sequence represented by SEQ ID NO:1;

(b) a DNA comprising the nucleotide sequence represented by SEQ ID NO:2;

(c) a DNA which hybridizes with the DNA consisting of the nucleotide sequence represented by SEQ ED NO:1 under stringent conditions and encodes a protein having α1,6-fucosyltransferase activity;

(d) a DNA which hybridizes with the DNA consisting of the nucleotide sequence represented by SEQ ID NO:2 under stringent conditions and encodes a protein having α1,6-fucosyltransferase activity.

(13) The cell according to (11), wherein the α1,6-fucosyltransferase is a protein selected from the group consisting of the following (a), (b), (c), (d), (e) and (f):

(a) a protein comprising the amino acid sequence represented by SEQ ID NO:23;

(b) a protein comprising the amino acid sequence represented by SEQ ID NO:24;

(c) a protein which consists of an amino acid sequence in which at least one amino acid is deleted, substituted, inserted and/or added in the amino acid sequence represented by SEQ ID NO:23 and has α1,6-fucosyltransferase activity;

(d) a protein which consists of an amino acid sequence in which at least one amino acid is deleted, substituted, inserted and/or added in the amino acid sequence represented by SEQ ID NO:24 and has α1,6-fucosyltransferase activity;

(e) a protein which consists of an amino acid sequence having a homology of at least 80% with the amino acid sequence represented by SEQ ID NO:23 and has α1,6-fucosyltransferase activity;

(f) a protein which consists of an amino acid sequence having a homology of at least 80% with the amino acid sequence represented by SEQ ID NO:24 and has α1,6-fucosyltransferase activity.

(14) The cell according to any one of (3) to (13), wherein the enzyme activity is decreased or deleted by a technique selected from the group consisting of the following (a), (b), (c), (d) and (e):

(a) a gene disruption technique targeting a gene encoding the enzyme;

(b) a technique for introducing a dominant negative mutant of a gene encoding the enzyme;

(c) a technique for introducing mutation into the enzyme;

(d) a technique for inhibiting transcription or translation of a gene encoding the enzyme;

(e) a technique for selecting a cell line resistant to a lectin which recognizes a sugar chain in which 1-position of fucose is bound to 6-position of N-acetylglucosamine in the reducing end through α-bond in the complex N-glycoside-linked sugar chain.

(15) The cell according to any one of (1) to (14), which is resistant to at least a lectin which recognizes a sugar chain in which 1-position of fucose is bound to 6-position of N-acetylglucosamine in the reducing end through α-bond in the complex N-glycoside-linked sugar chain.

(16) The cell according to any one of (1) to (15), which is a cell selected from the group consisting of the following (a) to (j):

(a) a CHO cell derived from a Chinese hamster ovary tissue;

(b) a rat myeloma cell line, YB2/3HLP2.G11.16Ag.20 cell;

(c) a mouse myeloma cell line, NS0 cell;

(d) a mouse myeloma cell line, SP2/0-Ag14 cell;

(e) a BHK cell derived from a Syrian hamster kidney tissue;

a monkey COS cell;

(g) an antibody-producing hybridoma cell;

(h) a human leukemia cell line, Namalwa cell;

(i) an embryonic stem cell;

(j) a fertilized egg cell.

(17) A transgenic non-human animal or plant or the progenies thereof into which an antibody molecule which specifically binds to CD20 and has complex N-glycoside-linked sugar chains bound to the Fc region is introduced, which produces an antibody composition comprising the antibody molecule, wherein among the total complex M-glycoside-linked sugar chains bound to the Fc region in the composition, the ratio of a sugar chain in which fucose is not bound to N-acetylglucosamine in the reducing end in the sugar chain is 20% or more.

(18) The transgenic non-human animal or plant or the progenies thereof according to (17), wherein the sugar chain in which fucose is not bound to N-acetylglucosamine is a sugar chain in which 1-position of the fucose is not bound to 6-position of N-acetylglucosamine in the reducing end through α-bond in the N-glycoside-linked sugar chain.

(19) The transgenic non-human animal or plant or the progenies thereof according to (17) or (18), wherein a genome is modified such that the activity of an enzyme relating to the synthesis of an intracellular sugar nucleotide, GDP-fucose and/or the activity of an enzyme relating to the modification of a sugar chain in which 1-position of fucose is bound to 6-position of N-acetylglucosamine in the reducing end through α-bond in the N-glycoside-linked sugar chain is decreased,

(20) The transgenic non-human animal or plant or the progenies thereof according to (17) or (18), wherein a gene encoding the enzyme relating to the synthesis of an intracellular sugar nucleotide, GDP-fucose, and/or a gene encoding the enzyme relating to the modification of a sugar chain in which 1-position of fucose is bound to 6-position of N-acetylglucosamine in the reducing end through α-bond in the N-glycoside-linked sugar chain is knocked out.

(21) The transgenic non-human animal or plant or the progenies thereof according to (19) or (20), wherein the enzyme relating to the synthesis of an intracellular sugar nucleotide, GDP-fucose is an enzyme selected from the group consisting of the following (a), (b) and (c):

(a) GMD (GDP-mannose 4,6-dehydratase);

(b) Fx (GDP-keto-6-deoxymannose 3,5-epimerase, 4-reductase);

(c) GFPP (GDP-beta-L-fucose pyrophosphorylase).

(22) The transgenic non-human animal or plant or the progenies thereof according to (21), wherein the GMD is a protein encoded by a DNA of the following (a) or (b):

(a) a DNA comprising the nucleotide sequence represented by SEQ ID NO:41;

(b) a DNA which hybridizes with the DNA consisting of the nucleotide sequence represented by SEQ ID NO:41 under stringent conditions and encodes a protein having GMD activity.

(23) The transgenic non-human animal or plant or the progenies thereof according to (21), wherein the Fx is a protein encoded by a DNA of the following (a) or (b):

(a) a DNA comprising the nucleotide sequence represented by SEQ ID NO:48;

(b) a DNA which hybridizes with the DNA consisting of the nucleotide sequence represented by SEQ ID NO:48 under stringent conditions and encodes a protein having Fx activity.

(24) The transgenic non-human animal or plant or the progenies thereof according to (21), wherein the GFPP is a protein encoded by a DNA of the following (a) or (b):

(a) a DNA comprising the nucleotide sequence represented by SEQ ID NO:51;

(b) a DNA which hybridizes with the DNA consisting of the nucleotide sequence represented by SEQ ID NO:51 under stringent conditions and encodes a protein having GFPP activity.

(25) The transgenic non-human animal or plant or the progenies thereof according to (19) or (20), wherein the enzyme relating to the modification of a sugar chain in which 1-position of fucose is bound to 6-position of N-acetylglucosamine in the reducing end through α-bond in the N-glycoside-linked sugar chain is α1,6-fucosyltransferase.

(26). The transgenic non-human animal or plant or the progenies thereof according to (25), wherein the α1,6-fucosyltransferase is a protein encoded by a DNA selected from the group consisting of the following (a), (b), (c) and (d):

(a) a DNA comprising the nucleotide sequence represented by SEQ ID NO:1;

(b) a DNA comprising the nucleotide sequence represented by SEQ ID NO:2;

(c) a DNA which hybridizes with the DNA consisting of the nucleotide sequence represented by SEQ ID NO:1 under stringent conditions and encodes a protein having α1,6-fucosyltransferase activity;

(d) a DNA which hybridizes with the DNA consisting of the nucleotide sequence represented by SEQ ID NO:2 under stringent conditions and encodes a protein having α1,6-fucosyltransferase activity.

(27) The transgenic non-human animal or plant or the progenies thereof according to any one of (17) to (26), wherein the transgenic non-human animal is an animal selected from the group consisting of cattle, sheep, goat, pig, horse, mouse, rat, fowl, monkey and rabbit.

(28) The cell according to any one of (1) to (16), wherein the antibody molecule is a molecule selected from the group consisting of (a), (b), (c) and (d):

(a) a human antibody;

(b) a humanized antibody,

(c) an antibody fragment comprising an Fc region of (a) or (b);

(d) a fusion protein comprising an Fc region of (a) or (b).

(29) The cell according to any one of (1) to (16) and (28), wherein the antibody molecule belongs to an IgG class.

(30) The cell according to any one of (1) to (16), (28) and (29), wherein the antibody molecule comprises complementarity determining regions 1, 2 and 3 of an antibody light chain variable region comprising the amino acid sequences represented by SEQ ID NOs:5, 6 and 7, respectively, and/or complementarity determining regions 1, 2 and 3 of an antibody heavy chain comprising the amino acid sequences represented by SEQ ID NOs:8, 9 and 10, respectively.

(31) The cell according to any one of (1) to (16), (28), (29) and (30), wherein the antibody molecule comprises a light chain variable region comprising the amino acid sequence represented by SEQ ID NO:12 and/or a heavy chain variable region comprising the amino acid sequence represented by SEQ ID NO 14.

(32) The transgenic non-human animal or plant or the progenies thereof according to any one of (17) to (27), wherein the antibody molecule is a molecule selected from the group consisting of (a), (b), (c) and (d):

(a) a human antibody;

(b) a humanized antibody;

(c) an antibody fragment comprising an Fc region of (a) or (b);

(d) a fusion protein comprising an Fc region of (a) or (b).

(33) The transgenic non-human animal or plant or the progenies thereof according to any one of (17) to (27) and (32), wherein the antibody molecule belongs to an IgG class.

(34) The transgenic non-human animal or plant or the progenies thereof according to any one of (17) to (27), (32) and (33), wherein the antibody molecule comprises complementarity determining regions 1, 2 and 3 of an antibody light chain variable region comprising the amino acid sequences represented by SEQ ID NOs:5, 6 and 7, respectively, and/or complementarity determining regions 1, 2 and 3 of an antibody heavy chain comprising the amino acid sequences represented by SEQ ID NOs:8, 9 and 10, respectively.

(35) The transgenic non-human animal or plant or the progenies thereof according to any one of (17) to (27), (32), (33) and (34), wherein the antibody molecule comprises a light chain variable region comprising the amino acid sequence represented by SEQ ID NO:12 and/or a heavy chain variable region comprising the amino acid sequence represented by SEQ ID NO:14.

(36) An antibody composition which is produced by the cell according to any one of (1) to (16) and (28) to (31).

(37) An antibody composition which is obtainable by rearing the transgenic non-human animal or plant or the progenies thereof according to any one of (17) to (27) and (32) to (35).

(38) An antibody composition comprising an antibody molecule which specifically binds to CD20 and has complex N-glycoside-linked sugar chains bound to the Fc region, wherein among the total complex N-glycoside-linked sugar chains bound to the Fc region in the composition, the ratio of a sugar chain in which fucose is not bound to N-acetylglucosamine in the reducing end in the sugar chain is 20% or more.

(39) The antibody composition according to (38), wherein the sugar chain to which fucose is not bound is a complex N-glycoside linked sugar chain in which 1-position of fucose is not bound to 6-position of N-acetylglucosamine in the reducing end through α-bond.

(40) The antibody composition according to (38), wherein the antibody molecule is a molecule selected from the group consisting of (a), (b), (c) and (d):

(a) a human antibody;

(b) a humanized antibody;

(c) an antibody fragment comprising an Fc region of (a) or (b);

(d) a fusion protein comprising an Fc region of (a) or (b).

(41) The antibody composition according to any one of (38) to (40), wherein the antibody molecule belongs to an IgG class.

(42) The antibody composition according to any one of (38) to (41), wherein the antibody molecule comprises complementarity determining regions 1, 2 and 3 of an antibody light chain variable region comprising the amino acid sequences represented by SEQ ID NOs:5, 6 and 7, respectively, and/or complementarity determining regions 1, 2 and 3 of an antibody heavy chain comprising the amino acid sequences represented by SEQ ID NOs:8, 9 and 10, respectively.

(43) The antibody composition according to any one of (38) to (42), wherein the antibody molecule comprises a light chain variable region comprising the amino acid sequence represented by SEQ ID NO:12 and/or a heavy chain variable region comprising the amino acid sequence represented by SEQ ID NO:14.

(44) A process for producing the antibody composition according to any one of (36) and (38) to (43), which comprises culturing the cell according to any one of (1) to (16) and (28) to (31) to form and accumulate the antibody composition in the culture; and recovering the antibody composition from the culture.

(45) A process for producing the antibody composition according to any one of (36) and (38) to (43), which comprises rearing the transgenic non-human animal or plant or the progenies thereof according to any one of (17) to (27) and (32) to (35); isolating tissue or body fluid from the reared animal or plant; and recovering the antibody composition from the isolated tissue or body fluid.

(46) A medicament which comprises the antibody composition according to any one of (36) to (43) as an active ingredient.

(47) An agent for treating diseases relating to CD20, which comprises the antibody composition according to any one of (36) to (43) as an active ingredient.

(48) The agent according to (47), wherein the disease relating to CD20 is a cancer or an immunological disease.

### BRIEF EXPLANATION OF THE DRAWINGS

FIG. 1 shows a construction step of plasmid pBS-2B8L.

FIG. 2 shows a construction step of plasmid pBS-2B8Hm.

FIG. 3 shows a construction step of plasmid pKANTEX2B8P.

FIG. 4 shows a result of measurement of the activity of purified anti-CD20 chimeric antibody KM3065 and Rituxan™ to bind to a human CD20-expressing cell, Raji cell while changing the concentration of the antibodies by using the immunofluorescent method. The ordinate and the abscissa show the relative fluorescence intensity at each concentration and the antibody concentration, respectively. “▪” and “∘” show the activities of Rituxan™ and KM3065, respectively.

FIG. 5 shows a result of measurement of the activity of purified anti-CD20 chimeric antibody XM3065 and Rituxan™ to bind to a human CD20-negative cell, CCRF—CEM cell, using the immunofluorescent method.

FIG. 6 shows ADCC activity of purified anti-CD20 chimeric antibody KM3065 and Rituxan™ to a human CD20-expressing cell. In FIGS. 6A, 6B and 6C, Raji cell, Ramos cell and WIL2-S were used as the target cell. The ordinate and the abscissa show the cytotoxic activity and the antibody concentration. “▪” and “∘” show the activities of Rituxan™ and KM3065, respectively.

FIG. 7 shows elution patterns obtained by preparing PA-modified sugar chains from purified anti-CD20 chimeric antibody KM3065 and Rituxan™ and analyzing them by reverse phase HPLC. The ordinate and the abscissa show the relative fluorescence intensity and the elution time, respectively.

FIG. 8 shows construction of a plasmid CHfFUT8-pCR2.1.

FIG. 9 shows construction of a plasmid ploxPPuro.

FIG. 10 shows construction of a plasmid pKOFUT8gE2-1.

FIG. 11 shows construction of a plasmid pKOFUT8gE2-2.

FIG. 12 shows construction of a plasmid pscFUT8gE2-3.

FIG. 13 shows construction of a plasmid pKOFUT8gE2-3.

FIG. 14 shows construction of a plasmid pKOFUT8gE2-4.

FIG. 15 shows construction of a plasmid pKOFUT8gE2-5.

FIG. 16 shows construction of a plasmid pKOFUT8Puro.

FIG. 17 shows a result of measurement of the binding activity of an anti-CD20 chimeric antibody R92-3-1 produced by lectin-resistant CHO/DG44 cell while changing the concentration of the antibody using the immunofluorescent method. The ordinate and the abscissa show the relative fluorescence intensity at each concentration and the antibody concentration, respectively. “▪” and “∘” show the activities of Rituxan™ and R92-3-1, respectively.

FIG. 18 shows a result of the evaluation of ADCC activity of the anti-CD20 chimeric antibody R92-3-1 produced by lectin-resistant CHO/DG44 cell, using Raji cell as the target cell. The ordinate and the abscissa show the cytotoxic activity on the target cell and the antibody concentration, respectively. “▪” and “∘” show the activities of Rituxan™ and R92-3-1, respectively.

FIG. 19 shows an elution pattern obtained by reverse phase HPLC analysis of a PA-modified sugar chain prepared from the anti-CD20 chimeric antibody R92-3-1 produced by lectin-resistant CHO/DG44 cell. The ordinate and the abscissa show the relative fluorescence intensity and the elution time, respectively. Analytical conditions of the reverse phase HPLC, identification of the sugar chain structure and calculation of the ratio of sugar chains to which α1,6-fucose was not bound were carried out in the same manner as in Example 3.

FIG. 20 shows a construction step of a plasmid CHO-GMD prepared by introducing 5′-terminal of a clone 34-2 into 5′-terminal of a CHO cell-derived GMD cDNA clone 22-8.

FIG. 21 shows elution patterns obtained by reverse phase BPLC analysis of PA-modified sugar chains prepared from three anti-CD20 chimeric antibodies. The ordinate and abscissa show the relative fluorescence intensity and the elution time, respectively. Analytical conditions of the reverse phase HPLC, identification of the sugar chain structure and calculation of the ratio of sugar chains to which α1,6-fucose was not bound were carried out in the same manner as in Example 3.

FIG. 22 shows a result of the measurement of the CD20-expressing cell-binding activity against five anti-CD20 chimeric antibodies having a different ratio of antibody molecules to which an α1,6-fucose-free sugar chain bound while changing the concentration of the antibodies using the immunofluorescent method. The ordinate and the abscissa show the binding activity to CD20 and the antibody concentration, respectively. “□”, “▪”, “Δ”, “▴” and “∘” show the activities of an anti-CD20 chimeric antibody (96%), an anti-CD20 chimeric antibody (44%), an anti-CD20 chimeric antibody (35%), an anti-CD20 chimeric antibody (26%) and an anti-CD20 chimeric antibody (6%), respectively.

FIG. 23 shows a result of the measurement of ADCC activity of anti-CD20 chimeric antibodies having a different ratio of antibody molecules to which an α1,6-fucose-free sugar chain is bound against WIL2-S cell. It shows a result measured by the ⁵¹Cr method using effector cells of donor A. The ordinate and the abscissa show the cytotoxic activity and the antibody concentration, respectively. “□”, “▪”, “Δ”, “▴” and “∘” show the activities of an anti-CD20 chimeric antibody (96%), an anti-CD20 chimeric antibody (44%), an anti-CD20 chimeric antibody (35%), an anti-CD20 chimeric antibody (26%) and an anti-CD20 chimeric antibody (6%), respectively.

FIG. 24 shows a result of the measurement of ADCC activity of anti-CD20 chimeric antibodies having a different ratio of antibody molecules to which a α1,6-fucose-free sugar chain is bound against Raji cell. It shows a result measured by the LDH method using effector cells of donor B. The ordinate and the abscissa show the cytotoxic activity and the antibody concentration. “□”, “▪”, “Δ”, “▴” and “∘” show the activities of an anti-CD20 chimeric antibody (96%), an anti-CD20 chimeric antibody (44%), an anti-CD20 chimeric antibody (35%), an anti-CD20 chimeric antibody (26%) and an anti-CD20 chimeric antibody (6%), respectively.

FIG. 25 shows an elution pattern obtained by separating anti-CD20 chimeric antibody KM3065 using a column immobilized with lectin having affinity for sugar chains containing bisecting GlcNAc. The ordinate and the abscissa show absorbance at 280 nm and the elution time, respectively. \mathring{1} to \mathring{4} show elution positions of fractions \mathring{1} to \mathring{4}.

FIG. 26 shows elution patterns of fractions \mathring{1} to \mathring{4} separated using a column immobilized with lectin having affinity for sugar chains containing bisecting GlcNAc and the PA-modified sugar chains prepared from anti-CD20 chimeric antibody KM3065 before the separation, each obtained by reverse phase HPLC analysis. The upper and left drawing, the upper and right drawing, the middle and left drawing, the middle and right drawing and the lower and left drawing show the elution patterns of KM3065 before the separation, fraction \mathring{1}, fraction \mathring{2}, fraction \mathring{3} and fraction \mathring{4}, respectively. The ordinate and the abscissa show the relative fluorescence intensity and the elution time, respectively. In the drawing, the peak painted out in black shows antibody-derived PA-modified sugar chains, and “*” shows PA-modified sugar chains having bisecting GlcNAc.

FIG. 27 shows ADCC activity of fractions \mathring{1} to \mathring{4} separated using a column immobilized with lectin having affinity for sugar chains containing bisecting GlcNAc and the anti-CD20 chimeric antibody KM3065 before the separation, against Raji cell. It shows a result of the measurement by the LDH method using effector cells derived from a healthy donor. The ordinate and the abscissa show the cytotoxicity and the antibody concentration, respectively. “●”, “∘”, “Δ”, “⋄”, “♦” and × show the activities of KM3065 before separation, fraction \mathring{1}, fraction \mathring{2}, fraction \mathring{3} and fraction \mathring{4}, Rituxan™ and no antibody-added case.

### DETAILED DESCRIPTION OF THE INVENTION

The cell of the present invention may be any cell, so long as the cell produces an antibody composition comprising an antibody molecule which specifically binds to CD20 and has complex N-glycoside-linked sugar chains bound to the Fc region, wherein among the total complex N-glycoside-linked sugar chains bound to the Fc region in the composition, the ratio of a sugar chain in which fucose is not bound to N-acetylglucosamine in the reducing end in the sugar chain is 20% or more.

In the present invention, CD20 is a cell surface membrane protein of about 35 kDa which is also called B1 or Bp35, and includes a protein represented by the amino acid sequence represented by SEQ ID NO:4, and a protein which comprises an amino acid sequence in which one or several amino acids are substituted, deleted, inserted and/or added in the amino acid sequence represented by SEQ ID NO:4 and has properties which are substantially similar to those of CD20.

The protein which comprises an amino acid sequence in which one or several amino acids are substituted, deleted, inserted and/or added in the amino acid sequence represented by SEQ ID NO:4 and has substantially similar activities to CD20 can be obtained e.g., by introducing a site-directed mutation into a DNA encoding a protein having the amino acid sequence represented by SEQ ID NO:4, using the site-directed mutagenesis described in, e.g., Molecular Cloning, A Laboratory Manual, Second Edition, Cold Spring Harbor Laboratory Press (1989) (hereinafter referred to as “Molecular Cloning, Second Edition”); Current Protocols in Molecular Biology, John Wiley & Sons, 1987-1997 (hereinafter referred to as “Current Protocols in Molecular Biology”); Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci., USA, C, 488 (1985); and the like. The number of amino acids to be deleted, substituted, inserted and/or added is one or more, and the number is not particularly limited, but is a number which can be deleted, substituted or added by a known technique such as the site-directed mutagenesis, e.g., it is 1 to several tens, preferably 1 to 20, more preferably 1 to 10, and most preferably 1 to 5.

Also, in order to maintain the CD20 activity of the protein to be used in the present invention, it has 80% or more, preferably 85% or more, more preferably 90% or more, still more preferably 95% or more, far more preferably 97% or more, and most preferably 99% or more, of homology with the amino acid sequence represented by SEQ ID NO:4 when calculated using an analyzing soft such as BLAST [J. Mol. Biol., 215, 403 (1990)], FASTA [Methods in Enzymology, 183, 63 (1990)] or the like.

In the present invention, as the sugar chain which binds to the Fc region of an antibody molecule, mentioned is an N-glycoside-linked sugar chain. As the N-glycoside-linked sugar chain, mentioned is a complex type sugar chain in which the non-reducing end side of the core structure has one or plural parallel branches of galactose-N-acetylglucosamine (hereinafter referred to as “Gal-GlcNAc”) and the non-reducing end side of Gal-GlcNAc has a structure such as sialic acid, bisecting N-acetylglucosamine or the like.

In an antibody, the Fc region has positions to which each of two N-glycoside-linked sugar chains is bound described below. Accordingly, two sugar chains are bound per one antibody molecule. Since the N-glycoside-linked sugar chain which binds to an antibody includes any sugar chain represented by the following structural formula (I), there are a number of combinations of sugar chains for the two N-glycoside-linked sugar chains which bind to the antibody. Accordingly, identity of substances can be judged from the viewpoint of the sugar structure bound to the Fc region.

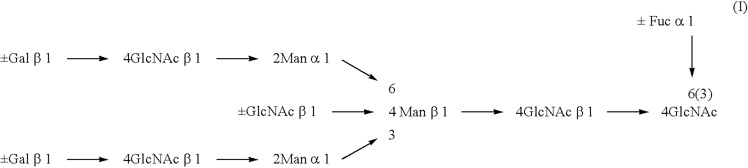

In the present invention, the composition which comprises an antibody molecule having complex N-glycoside-linked sugar chains in the Fc region (hereinafter referred to as “antibody composition of the present invention”) may comprise an antibody having the same sugar chain structures or an antibody having different sugar chain structures, so long as the effect of the present invention is obtained from the composition.

In the present invention, “the ratio of a sugar chain in which fucose is not bound to N-acetylglucosamine in the reducing end in the sugar chain among the total complex N-glycoside-linked sugar chains bound to the Fc region contained in the antibody composition” means a ratio of the number of a sugar chain in which fucose is not bound to N-acetylglucosamine in the reducing end in the sugar chain to the total number of the complex N-glycoside-linked sugar chains bound to the Fc region contained in the composition.

In the present invention, “the sugar chain in which fucose is not bound to N-acetylglucosamine in the reducing end in the complex X-glycoside-linked sugar chain” means a sugar chain in which 1-position of the fucose is not bound to N-acetylglucosamine in the reducing end through α-bond in the complex N-glycoside-linked sugar chain Examples include a complex N-glycoside-linked sugar chain in which 1-position of fucose is not bound to 6-position of N-acetylglucosamine through c-bond.

The ratio of a sugar chain in which fucose is not bound to N-acetylglucosamine in the reducing end in the sugar chain among the total complex N-glycoside-linked sugar chains binding to the Fc region contained in the antibody composition of the present invention is preferably 20% or more, more preferably 25% or more, still more preferably 30% or more, far preferably 40% or more, and most preferably 50% or more. The antibody composition having this ratio of a sugar chain has high ADCC activity.

As the antibody concentration is decreased, the ADCC activity is decreased accordingly. However, high ADCC activity can be obtained even though the antibody concentration is low, so long as the ratio of a sugar chain in which fucose is not bound to N-acetylglucosamine in the reducing end in the sugar chain is 20% or more.

The ratio of a sugar chain in which fucose is not bound to N-acetylglucosamine in the reducing end in the sugar chain contained in the composition which comprises an antibody molecule having complex N-glycoside-linked sugar chains in the Fe region can be determined by releasing the sugar chain from the antibody molecule using a known method such as hydrazinolysis, enzyme digestion or the like [Biochemical Experimentation Methods 23—Method for Studying Glycoprotein Sugar Chain (Japan Scientific Societies Press), edited by Peiko Takahashi (1989)], carrying out fluorescence labeling or radioisotope labeling of the released sugar chain, and then separating the labeled sugar chain by chromatography. Alternatively, the released sugar chain can be determined by analyzing it with the HPAED-PAD method [J Liq. Chromatogr., 6, 1577 (1983)].

Furthermore, the cell of the present invention includes a cell which produces the composition of the present invention, wherein the activity of an enzyme relating to the synthesis of an intracellular sugar nucleotide, GDP-fucose and/or the activity of an enzyme relating to the modification of a sugar chain in which 1-position of fucose is bound to 6-position of N-acetylglucosamine in the reducing end through α-bond in the complex N-glycoside-linked sugar chain is decreased or deleted.

In the present invention, the enzyme relating to the synthesis of an intracellular sugar nucleotide, GDP-fucose, may be any enzyme, so long as it is an enzyme relating to the synthesis of the intracellular sugar nucleotide, GDP-fucose, as a supply source of fucose to a sugar chain. The enzyme relating to the synthesis of an intracellular sugar nucleotide, GDP-fucose, includes an enzyme which has influence on the synthesis of the intracellular sugar nucleotide, GDP-fucose.

The enzyme which has influence on the synthesis of an intracellular sugar nucleotide, GDP-fucose, includes an enzyme which has influence on the activity of the enzyme relating to the synthesis of the intracellular sugar nucleotide, GDP-fucose, and an enzyme which has influence on the structure of substance used as a substrate of the enzyme.

The intracellular sugar nucleotide, GDP-fucose, is supplied by a de novo synthesis pathway or a salvage synthesis pathway. Thus, all enzymes relating to the synthesis pathways are included in the enzyme relating to the synthesis of an intracellular sugar nucleotide, GDP-fucose.

The enzyme relating to the de novo synthesis pathway of the intracellular sugar nucleotide, GDP-fucose, include GDP-mannose 4,6-dehydratase (hereinafter referred to as “GMD”), GDP-keto-6-deoxymannose 3,5-epimerase 4,6-reductase (hereinafter refeed to as “Fx”) and the like.

The enzyme relating to the salvage synthesis pathway of the intracellular sugar nucleotide, GDP-fucose, include GDP-beta-L-fucose pyrophosphorylase (hereinafter referred to as “GFPP”), fucokinase and the like.

In the present invention, the GMD includes:

a protein encoded by a DNA of the following (a) or (b):

(a) a DNA comprising the nucleotide sequence represented by SEQ ID NO:41;

(b) a DNA which hybridizes with the DNA consisting of the nucleotide sequence represented by SEQ ID NO:41 under stringent conditions and encodes a protein having GMD activity,

(c) a protein comprising the amino acid sequence represented by SEQ ID NO:61,

(d) a protein which consists of an amino acid sequence in which at least one amino acid is deleted, substituted, inserted and/or added in the amino acid sequence represented by SEQ ID NO:61 and has GMD activity, and

(e) a protein which consists of an amino acid sequence having a homology of at least 80% with the amino acid sequence represented by SEQ ID NO:61 and has GMD activity.

Also, the DNA encoding the amino acid sequence of GMD includes a DNA comprising the nucleotide sequence represented by SEQ ID NO:41 and a DNA which hybridizes with the DNA consists of the nucleotide sequence represented by SEQ ID NO:41 under stringent conditions and encodes an amino acid sequence having GMD activity.

In the present invention, the Fx includes:

a protein encoded by a DNA of the following (a) or (b):

(a) a DNA comprising the nucleotide sequence represented by SEQ ID NO:48;

(b) a DNA which hybridizes with the DNA consists of the nucleotide sequence represented by SEQ ID NO:48 under stringent conditions and encodes a protein having Fx activity,

(c) a protein comprising the amino acid sequence represented by SEQ ID NO:62,

(d) a protein which consists of an amino acid sequence in which at least one amino acid is deleted, substituted, inserted and/or added in the amino acid sequence represented by SEQ ID NO:62 and has Fx activity, and

(e) a protein which consists of an amino acid sequence having a homology of at least 80% with the amino acid sequence represented by SEQ ID NO:62 and has Fx activity.

Also, the DNA encoding the amino acid sequence of Fx includes a DNA comprising the nucleotide sequence represented by SEQ ID NO:48 and a DNA which hybridizes with the DNA consists of the nucleotide sequence represented by SEQ ID NO:48 under stringent conditions and encodes an amino acid sequence having Fx activity.

In the present invention, the GFPP includes:

a protein encoded by a DNA of the following (a) or (b):

(a) a DNA comprising the nucleotide sequence represented by SEQ ID NO:51;

(b) a DNA which hybridizes with the DNA consists of the nucleotide sequence represented by SEQ ID NO:51 under stringent conditions and encodes a protein having GFPP activity,

(c) a protein comprising the amino acid sequence represented by SEQ ID NO:63,

(d) a protein which comprises an amino acid sequence in which at least one amino acid is deleted, substituted, inserted and/or added in the amino acid sequence represented by SEQ ID NO:63 and has GFPP activity, and

(e) a protein which consists of an amino acid sequence having a homology of at least 80% with the amino acid sequence represented by SEQ ID NO:63 and has GFPP activity.

Also, the DNA encoding the amino acid sequence of GFPP include a DNA comprising the nucleotide sequence represented by SEQ ID NO:51 and a DNA which hybridizes with the DNA consisting of the nucleotide sequence represented by SEQ ID NO:51 under stringent conditions and encodes an amino acid sequence having Fx activity.

In the present invention, the enzyme relating to the modification of a sugar chain in which 1-position of fucose is bound to 6-position of N-acetylglucosamine in the reducing end through α-bond in the complex N-glycoside-linked sugar chain includes any enzyme, so long as it is an enzyme relating to the reaction of binding of 1-position of fucose to 6-position of N-acetylglucosamine in the reducing end through α-bond in the complex N-glycoside-linked sugar chain.

“The enzyme relating to the reaction of binding of 1-position of fucose to 6-position of N-acetylglucosamine in the reducing end through α-bond in the complex N-glycoside-linked sugar chain” means an enzyme which has influence in the reaction of binding of 1-position of fucose to 6-position of N-acetylglucosamine in the reducing end through α-bond in the complex N-glycoside-linked sugar chain.

The enzyme relating to the reaction of binding of 1-position of fucose to 6-position of N-acetylglucosamine in the reducing end through α-bond in the complex N-glycoside-linked sugar chain include α1,6-fucosyltransferase and α-L-fucosidase.

Also, examples include an enzyme which has influence on the activity the enzyme relating to the reaction of binding of 1-position of fucose to 6-position of N-acetylglucosamine in the reducing end through α-bond in the complex N-glycoside-linked sugar chain and an enzyme which has influence on the structure of substances as the substrate of the enzyme.

In the present invention, the α1,6-fucosyltransferase includes:

a protein encoded by a DNA of the following (a), (b), (c) or (d):

(a) a DNA comprising the nucleotide sequence represented by SEQ ID NO:1;

(b) a DNA comprising the nucleotide sequence represented by SEQ ID NO:2;

(c) a DNA which hybridizes with the DNA consisting of the nucleotide sequence represented by SEQ ID NO:1 under stringent conditions and encodes a protein having α1,6-fucosyltransferase activity;

(d) a DNA which hybridizes with the DNA consisting of the nucleotide sequence represented by SEQ ID NO:2 under stringent conditions and encodes a protein having α1,6-fucosyltransferase activity;

(e) a protein comprising the amino acid sequence represented by SEQ ID NO:23,

(f) a protein comprising the amino acid sequence represented by SEQ ID NO:24,

(g) a protein which consisting of an amino acid sequence in which at least one amino acid is deleted, substituted, inserted and/or added in the amino acid sequence represented by SEQ ID NO:23 and has α1,6-fucosyltransferase activity,

(h) a protein which consisting of an amino acid sequence in which at least one amino acid is deleted, substituted, inserted and/or added in the amino acid sequence represented by SEQ ID NO:24 and has α1,6-fucosyltransferase activity,

(i) a protein which consisting of an amino acid sequence having a homology of at least 80% with the amino acid sequence represented by SEQ ID NO:23 and has α1,6-fucosyltransferase activity, and

(j) a protein which consisting of an amino acid sequence having a homology of at least 80% with the amino acid sequence represented by SEQ ID NO:24 and has α1,6-fucosyltransferase activity.

Also, the DNA encoding the amino acid sequence of α1,6-fucosyltransferase includes a DNA having the nucleotide sequence represented by SEQ ID NO:1 or 2 and a DNA which hybridizes with the DNA having the nucleotide sequence represented by SEQ ID NO:1 or 2 under stringent conditions and encodes an amino acid sequence having α1,6-fucosyltransferase activity.

In the present invention, “a DNA which hybridizes under stringent conditions” means a DNA obtained by a method such as colony hybridization, plaque hybridization or Southern blot hybridization using a DNA such as the DNA having the nucleotide sequence represented by SEQ ID NO:1, 2, 48, 51 or 41 or a partial fragment thereof as the probe. Specifically mentioned is a DNA which can be identified by carrying out hybridization at 65° C. in the presence of 0.7 to 1.0 M sodium chloride using a filter to which colony- or plaque-derived DNA fragments are immobilized, and then washing the filter at 65° C. using 0.1 to 2×SSC solution (composition of the 1×SSC solution comprising 150 mM sodium chloride and 15 mM sodium citrate). The hybridization can be carried out in accordance with the methods described, e.g., in Molecular Cloning, A Laboratory Manual, Second Edition., Cold Spring Harbor Laboratory Press (1989) (hereinafter referred to as “Molecular Cloning, Second Edition”), Current Protocols in Molecular Biology, John Wiley & Sons, 1987-1997 (hereinafter referred to as “Current Prolocols in Molecular Biology”); DNA Cloning 1: Core Techniques, A Practical Approach, Second Edition, Oxford University (1995); and the like. Examples of the hybridizable DNA include a DNA having at least 60% or more, preferably 700/0 or more, more preferably 80% or more, still more preferably 90% or more, far more preferably 95% or more, and most preferably 98% or more, of homology with the nucleotide sequence represented by SEQ ID NO:1, 2, 48, 51 or 41.

In the present invention, the protein which consists of an amino acid sequence in which at least one amino acid is deleted, substituted, inserted and/or added in the amino acid sequence represented by SEQ ID NO:23, 24, 61, 62 and 63 respectively and has α1,6-fucosyltransferase activity, GMD activity, Ex activity and GFPP activity can be obtained by introducing a site-directed mutation into a DNA encoding a protein having the amino acid sequence represented by SEQ ID NO:1, 2, 41, 48 and 51 respectively using the site-directed mutagenesis described, e.g., in Molecular Cloning, Second Edition; Current Protocols in Molecular Biology; Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985); and the like. The number of amino acids to be deleted, substituted, inserted and/or added is one or more, and the number is not particularly limited, but is a number which can be deleted, substituted or added by a known technique such as the site-directed mutagenesis, e.g., it is 1 to several tens, preferably 1 to 20, more preferably 1 to 10, and most preferably 1 to 5.

Also, each of proteins to be used in the present invention has at least 80% or more, preferably 85% or more, more preferably 90% or more, still more preferably 95% or more, far more preferably 97% or more, and most preferably 99% or more, of homology with the amino acid sequence represented by SEQ ID NO:23, 24, 61, 62 and 63 respectively, when calculated using an analyzing soft such as BLAST [J. Mol. Biol., 215, 403 (1990)], FASTA [Methods in Enzymology, 183, 63 (1990)] or the like so that it can maintain the α1,6-fucosyltransferase activity, GMD activity, Fx activity and GFPP activity, respectively.

Furthermore, as the method for obtaining the cell of the present invention, that is, the cell in which the activity of an enzyme relating to the synthesis of an intracellular sugar nucleotide, GDP-fucose, and/or the activity of an enzyme relating to the modification of a sugar chain wherein 1-position of fucose is bound to 6-position of N-acetylglucosamine in the reducing end through α-bond in the complex N-glycoside-linked sugar chain is decreased or deleted, any technique can be used, so long as it can decrease the enzyme activity of interest. The technique for decreasing or deleting the enzyme activity include:

(a) a gene disruption technique targeting a gene encoding the enzyme,

(b) a technique for introducing a dominant negative mutant of a gene encoding the enzyme,

(c) a technique for introducing mutation into the enzyme,

(d) a technique for inhibiting transcription and/or translation of a gene encoding the enzyme, and

(e) a technique for selecting a cell line resistant to a lectin which recognizes a sugar chain in which 1-position of fucose is bound to 6-position of N-acetylglucosamine in the reducing end through α-bond in the complex N-glycoside-linked sugar chain.

As the lectin which recognizes a sugar chain structure in which 1-position of fucose is bound to 6-position of N-acetylglucosamine in the reducing end through α-bond in the N-glycoside-linked sugar chain, any lectin which can recognize the sugar chain structure can be used. Examples include a Lens culmaris lectin LCA (lentil agglutinin derived from Lens culnaris), a pea lecin PSA (pea lectin derived from Pisum sativum), a broad bean lectin VFA (agglutinin derived from Vicia faba), and an Aleuria aurantia lectin AAL (lectin derived from Aleuria aurantia).

The host cell for producing the antibody composition of the present invention may be any host, so long as it can express an anti-CD20 antibody molecule, i.e., a host cell transfected with a gene encoding an anti-CD20 antibody molecule. Examples include a yeast, an animal cell, an insect cell, a plant cell and the like. Examples of the cells include those described below in item 1. Among animal cells, preferred are include a CHO cell derived from a Chinese hamster ovary tissue, a rat myeloma cell line YB2/3HL.P2.G11.16Ag.20 cell, a mouse myeloma cell line NS0 cell, a mouse myeloma SP2/0-Ag14 cell, a BHK cell derived from a syrian hamster kidney tissue, an antibody producing-hybridoma cell, a human leukemia cell line Namalwa cell, an embryonic stem cell, a fertilized egg cell and the like. Examples include a rat myeloma cell line YB2/3HL.P2.G11.16Ag.20 cell transformed clone KM3065 (FERM BP-7834) transfected with the anti-CD20 antibody gene of the present invention.

The transgenic non-human animal or plant or the progenies thereof are not limited, so long as it is a transgenic non-human animal or plant or progeny thereof which produces an antibody composition comprising an antibody molecule which specifically binds to CD20 and has complex N-glycoside-linked sugar chains bound to the Fc region, wherein among the total complex N-glycoside-linked sugar chains bound to the Fc region in the composition, the ratio of a sugar chain in which fucose is not bound to N-acetylglucosamine in the reducing end in the sugar chain is 20% or more, and into which a gene encoding the antibody molecule is introduced. The antibody-producing transgenic animal can be prepared by introducing a gene encoding an antibody which specifically binds to CD20 into ES cell of a mouse, transplanting the ES cell into an early stage embryo of other mouse and then developing it. The transgenic animal can be also prepared by introducing a gene encoding an antibody which specifically binds to CD20 into a fertilized egg and developing it.

The transgenic non-human animal include cattle, sheep, goat, pig, horse, mouse, rat, fowl, monkey, rabbit and the like.

In the present invention, the antibody molecule includes any molecule, so long as it comprises the Fc region of an antibody. Examples include an antibody, an antibody fragment, a fusion protein comprising an Fc region, and the like.

The antibody is a protein which is produced in the living body by immune response as a result of exogenous antigen stimulation and has an activity to specifically bind to the antigen. As the antibody, an antibody secreted by a hybridoma cell prepared from a spleen cell of an animal immunized with an antigen; an antibody prepared by a recombinant DNA technique, i.e., an antibody obtained by introducing an antibody gene-inserted antibody expression vector into a host cell; and the like are mentioned. Examples include an antibody produced by a hybridoma, a humanized antibody, a human antibody and the like.

As a hybridoma, a cell which is obtained by cell fusion between a B cell obtained by immunizing a mammal other than human with an antigen and a myeloma cell derived from mouse or the like and can produce a monoclonal antibody having the desired antigen specificity can be mentioned.

As the humanized antibody, a human chimeric antibody, a human complementarity determining region (hereinafter referred to as “CDR”)-grafted antibody and the like can be mentioned.

A human chimeric antibody is an antibody which comprises a non-human antibody heavy chain variable region (hereinafter referred to as “HV” or “VH”, the variable chain and the heavy chain being “V region” and “H chain”, respectively) and a non-human antibody light chain variable region (hereinafter referred to as “LV” or “VL”), a human antibody heavy chain constant region (hereinafter also referred to as “CH”) and a human antibody light chain constant region (hereinafter also referred to as “CL”). As the non-human animal, any animal such as mouse, rat hamster, rabbit or the like can be used, so long as a hybridoma can be prepared therefrom.

The human chimeric antibody can be produced by preparing cDNAs encoding VH and VL from a monoclonal antibody-producing hybridoma, inserting them into an expression vector for host cell having genes encoding human antibody CH and human antibody CL to thereby construct a vector for expression of human chimeric antibody, and then introducing the vector into a host cell to express the antibody.

As the CH of human chimeric antibody, any CH can be used, so long as it belongs to human immunoglobulin (hereinafter referred to as “hIg”). Those belonging to the hIgG class are preferable, and any one of the subclasses belonging to the hIgG class, such as hIgG1, hIgG2, hIgG3 and hIgG4, can be used. Also, as the CL of human chimeric antibody, any CL can be used, so long as it belongs to the hMg class, and those belonging to the κ class or λ class can also be used.

A human CDR-grafted antibody is an antibody in which amino acid sequences of CDRs of VH and VL of an antibody derived from a non-human animal are grafted into appropriate positions of VH and VL of a human antibody.

The human CDR-grafted antibody can be produced by constructing cDNAs encoding V regions in which CDRs of VH and VL of an antibody derived from a non-human animal are grafted into CDRs of VH and VL of a human antibody, inserting them into an expression vector for host cell having genes encoding human antibody CH and human antibody CL to thereby construct a vector for human CDR-grafted antibody expression, and then introducing the expression vector into a host cell to express the human CDR-grafted antibody.

As the CH of human CDR-grafted antibody, any CH can be used, so long as it belongs to the hIg, but those of the hIgG class are preferable, and any one of the subclasses belonging to the hIgG class, such as hIgG1, hIgG2, hIgG3 and hIgG4, can be used. Also, as the CL of human CDR-grafted antibody, any CL can be used, so long as it belongs to the hIg class, and those belonging to the κ class or λ class can also be used.

A human antibody is originally an antibody naturally existing in the human body, but it also includes antibodies obtained from a human antibody phage library, a human antibody-producing transgenic animal and a human antibody-producing transgenic plant, which are prepared based on the recent advance in genetic engineering, cell engineering and developmental engineering techniques.

The antibody existing in the human body can be obtained by isolating a human peripheral blood lymphocyte, immortalizing it by its infection with EB virus or the like, cloning it to obtain a lymphocyte capable of producing the antibody, culturing the lymphocyte, and isolating and purifying the antibody from the culture.

The human antibody phage library is a library in which antibody fragments such as Fab (fragment of antigen binding), a single chain antibody and the like are expressed on the phage surface by inserting a gene encoding an antibody prepared from a human B cell into a phage gene. A phage expressing an antibody fragment having the desired antigen binding activity can be recovered from the library, using its activity to bind to an antigen-immobilized substrate as the marker. The antibody fragment can be converted further into a human antibody molecule comprising two full H chains and two full L chains by recombinant DNA techniques.

An antibody fragment is a fragment which comprises the Fc region of an antibody. As the antibody fragment, an H chain monomer, an H chain dimer and the like can be mentioned.

A fusion protein comprising an Fc region includes a composition in which an antibody comprising the Fc region of an antibody or the antibody fragment is fused with a protein such as an enzyme, a cytokine or the like.

The antibody molecule of the present invention may be any antibody molecule, so long as it specifically binds to CD20. The antibody molecule is preferably an antibody molecule which specifically binds to CD20 and comprises complementarity determining regions 1, 2 and 3 of an antibody light chain variable region represented by the amino acid sequences represented by SEQ BD NOs:5, 6 and 7, respectively, and/or complementarity determining regions 1, 2 and 3 of an antibody heavy chain represented by the amino acid sequences represented by SEQ ID NOs:8, 9 and 10, respectively, and more preferably the antibody molecule which specifically binds to CD20 and comprises a light chain variable region represented by SEQ ID NO:12 and/or a heavy chain variable region represented by SEQ ID NO:14.

The medicament of the present invention includes a medicament which comprises, as an active ingredient, the antibody composition of the present invention, i.e., the composition comprising an anti-CD20 antibody molecule.

The diseases relating to CD20 includes cancers such as B cell lymphoma, inflammatory diseases, autoimmune disease and the like.

In the present invention, the ADCC activity is a cytotoxic activity in which an antibody bound to a cell surface antigen on a tumor cell and the like in the living body activate an effector cell through an Fc receptor existing on the antibody Fc region and effector cell surface and thereby obstruct the tumor cell and the like [Monoclonal Antibodies: Principles and Applications, Wiley-Liss, Inc., Chapter 2.1(1955)]. As the effector cell, a killer cell, a natural killer cell, an activated macrophage and the like can be mentioned.

The present invention is described below in detail.

1. Preparation of the Cell Which Produces the Antibody Composition of the Present Invention

The cell of the present invention can be prepared by preparing a host cell used for producing the antibody composition of the present invention according to the following techniques and transfecting a gene encoding an anti-CD20 antibody into the host cell according to the method described in the following item 3.

(1) Gene Disruption Technique Targeting at a Gene Encoding an Enzyme

The host cell used for producing the cell of the present invention can be prepared using a gene disruption technique by targeting a gene encoding an enzyme relating to the synthesis of an intracellular sugar nucleotide, GDP-fucose, or targeting a gene encoding an enzyme relating to the modification of a sugar chain wherein 1-position of fucose is bound to 6-position of N-acetylglucosamine in the reducing end through α-bond in the complex N-glycoside-linked sugar chain. As the enzyme relating to the synthesis of an intracellular sugar nucleotide, GDP-fucose, GMD, Fx, GFPP, fucokinase and the like can be mentioned. As the enzyme relating to the modification of a sugar chain wherein 1-position of fucose is bound to 6-position of N-acetylglucosamine in the reducing end through α-bond in the complex N-glycoside-linked sugar chain, α1,6-fucosyltransferase, α-L-fucosidase and the like can be mentioned.

The gene as used herein includes DNA and RNA.

As the gene disruption method, any method can be include, so long as it can disrupt the gene of the target enzyme As examples, an antisense method, a ribozyme method, a homologous recombination method, an RNA-DNA oligonucleotide method (hereinafter referred to as “RDO method”), an RNA interface method (hereinafter referred to as “RNAi method”), a method using retrovirus, a method using transposon, and the like can be mentioned. The methods are specifically described below.

(a) Preparation of the Host Cell for Preparing the Cell of the Present Invention by the Antisense Method or the Ribozyme Method

The host cell for preparing the cell of the present invention can be prepared by the antisense method or the ribozyme method described in Cell Technology, 12, 239 (1993); BIOTECHNOLOGY, 17, 1097 (1999); Hum. Mol. Genet., 5, 1083 (1995); Cell Technology, 13, 255 (1994); Proc. Natl. Acad. Sci. USA, 96, 1886 (1999); or the like, e.g., in the following manner by targeting a gene encoding an enzyme relating to the synthesis of an intracellular sugar nucleotide, GDP-fucose, and/or a gene encoding an enzyme relating to the modification of a sugar chain wherein 1-position of fucose is bound to 6-position of N-acetylglucosamine in the reducing end through o-bond in the complex N-glycoside-linked sugar chain.

A cDNA or a genomic DNA encoding an enzyme relating to the synthesis of an intracellular sugar nucleotide, GDP-fucose, or encoding an enzyme relating to the modification of a sugar chain wherein 1-position of fucose is bound to 6-position of N-acetylglucosamine in the reducing end through α-bond in the complex N-glycoside-linked sugar chain is prepared.

The nucleotide sequence of the prepared cDNA or genomic DNA is determined.

Based on the determined DNA sequence, an appropriate length of an antisense gene or ribozyme construct comprising a part of a DNA which encodes the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain is designed. The designed construct can further contain a part of non-translation region or an intron.

In order to express the antisense gene or the ribozyme in a cell, a recombinant vector is prepared by inserting a fragment or total length of the prepared DNA into downstream of the promoter of an appropriate expression vector.

A transformant is obtained by introducing the recombinant vector into a host cell suitable for the expression vector.

The cell of the present invention can be obtained by selecting a transformant using, as a marker, the activity of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain. The host cell for preparing the cell of the present invention can also be obtained by selecting a transformant based on the sugar chain structure of a glycoprotein on the cell membrane or the sugar chain structure of the produced antibody molecule.

As the host cell for preparing the cell of the present invention, any cell such as a yeast, an animal cell, an insect cell or a plant cell can be used, so long as it has a gene encoding the target enzyme relating to the synthesis of an intracellular sugar nucleotide, GDP-fucose, or a gene encoding the target enzyme relating to the modification of a sugar chain wherein 1-position of fucose is bound to 6-position of N-acetylglucosamine in the reducing end through α-bond in the complex N-glycoside-linked sugar chain. Examples include host cells described in the following item 3.

As the expression vector, a vector which is autonomously replicable in the host cell or can be integrated into the chromosome and comprises a promoter at such a position that the designed antisense gene or ribozyme can be transferred can be used. Examples include expression vectors described in the following item 3.

Regarding the method for introducing a gene into various host cells, the methods for introducing recombinant vectors suitable for various host cells, which are described in the following item 3, can be used.

The following method can be exemplified as the method for selecting a transformant using, a marker of the activity of an enzyme relating to the synthesis of an intracellular sugar nucleotide, GDP-fucose and/or the activity of an enzyme relating to the modification of a sugar chain wherein 1-position of fucose is bound to 6-position of N-acetylglucosamine in the reducing end through α-bond in the complex N-glycoside-linked sugar chain.

Method for Selecting Transformant:

The method for selecting a cell in which the activity of an enzyme relating to the synthesis of an intracellular sugar nucleotide, GDP-fucose and/or the activity of an enzyme relating to the modification of a sugar chain wherein 1-position of fucose is bound to 6-position of N-acetylglucosamine in the reducing end through α-bond in the complex N-glycoside-linked sugar chain is decreased include biochemical methods or genetic engineering techniques described in New Biochemical Experimentation Series 3-Sacchaides I, Giycoprozein (Tokyo Kagaku Dojin), edited by Japanese Biochemical society (1988), Cell Engineering, Supplement, Experimental Protocol Series, Glycobiology Experimental Protocol, Glycoprotein, Glycolipid and Proteoglycan (Shujun-sha), edited by Naoyuki Taniguchi, Akemi Suzuki, Kiyoshi Furukawa and Kazuyuki Sugawara (1996); Molecular Cloning, Second Edition; Current Protocols in Molecular Biology, and the like. The biochemical method includes a method in which the enzyme activity is evaluated using an enzyme-specific substrate and the like. The genetic engineering technique include the Northern analysis, RT-PCR and the like wherein the amount of mRNA of a gene encoding the enzyme is measured.

The method for selecting a transformant based on the sugar chain structure of a glycoprotein on the cell membrane includes methods described in the following item 1(5). The method for selecting a transformant based on the sugar chain structure of a produced antibody molecule includes methods described in the following items 4 and 5.

As the method for preparing cDNA encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain, the following method is exemplified.

Preparation Method of DNA:

A total RNA or mRNA is prepared from tissues or cells of various host cells.

A cDNA library is prepared from the prepared total RNA or mRNA.

Degenerative primers are produced based on the amino acid sequence of an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain, and a gene fragment encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through (α-bond in the complex N-glycoside-linked sugar chain is obtained by PCR using the prepared cDNA library as the template.

A DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain can be obtained by screening the cDNA library using the obtained gene fragment as a probe.

Regarding the mRNA of a human or non-human tissue or cell, a commercially available product (e.g., manufactured by Clontech) may be used or it may be prepared from a human or non-human animal tissue or cell in the following manner. Examples of the method for preparing a total RNA from a human or non-human animal tissue or cell include the guanidine thiocyanate-cesium trifluoroacetate method [Methods in Enzymology, 11, 3 (1987)], the acidic guanidine thiocyanate phenol chloroform (AGPC) method [Analytical Biochemistry, 162, 156 (1987); Experimental Medicine, 9, 1937 (1991)] and the like.

Also, the method for preparing mRNA from a total RNA as poly(A)⁺ RNA include an oligo(dT)-immobilized cellulose column method (Molecular Cloning, Second Edition) and the like.

In addition, mRNA can be prepared using a kit such as Fast Track mRNA Isolation Kit (manufactured by Invitrogen), Quick Prep mRNA Purification Kit (manufactured by Pharmacia) or the like.

A cDNA library is prepared from the prepared mRNA of a human or non-human animal tissue or cell. The method for preparing cDNA libraries include the methods described in Molecular Cloning, Second Edition; Current Protocols in Molecular Biology; A Laboratory Manual, Second Edition (1989); and the like, or methods using commercially available kits such as SuperScript Plasmid System for cDNA Synthesis and Plasmid Cloning (manufactured by Life Technologies), ZAPcDNA Synthesis Kit (manufactured by STRATAGEI) and the like.

As the cloning vector for preparing the cDNA library, any vector such as a phage vector, a plasmid vector or the like can be used, so long as it is autonomously replicable in Escherichia coli K12. Examples include ZAP Express [manufactured by STRATAGENE, Stategies, 5, 58 (1992)], pBluescript II SK(+) [Nucleic Acids Research, 17, 9494 (1989)], Lambda ZAP II (manufactured by STRATAGENE), λgt10 and λgt11 [DNA Cloning, A Practical Approach, 1, 49 (1985)], λTriplEx (manufactured by Clontech), λExCell (manufactured by Pharmacia), pT7T318U (manufactured by Pharmacia), pcD2 [Mol. Cell Biol., 3, 280(1983)], pUC18 [Gene, 33, 103 (1985)] and the like.

Any microorganism can be used as the host microorganism, and Escherichia coli is preferably used. Examples include Escherichia coli XL1-Blue MRF′ [manufactured by STRATAGENE, Strategies, 5, 81.(1992)], Escherichia coli C600 [Genetics, 39, 440 (1954)], Escherichia coli Y1088 [Science, 222, 778 (1983)], Escherichia coli Y1090 [Science, 22, 778 (1983)], Escherichia coli NM522 [J. Mol. Biol., 166, 1 (1983)], Escherichia coli K802 [J. Mol. Biol., 16, 118 (1966)], Escherichia coli JM105 [Gene, 38, 275 (1985)] and the like

The cDNA library may be used as such in the subsequent analysis, and in order to obtain a full length cDNA as efficient as possible by decreasing the ratio of an infull length cDNA, a cDNA library prepared using the oligo cap method developed by Sugano et al. [Gene, 138, 171 (1994); Gene, 200, 149 (1997); Protein, Nucleic Acid and Enzyme, 4, 603 (1996); Experimental Medicine, 11, 2491 (1993); cDNA Cloning (Yodo-sha) (1996); Methods for Preparing Gene Libraries (Yodo-sha) (1994)] may be used in the following analysis

Degenerative primers specific for the 5′-terminal and 3′-terminal nucleotide sequences of a nucleotide sequence presumed to encode the amino acid sequence are prepared based on the amino acid sequence of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain, and DNA is amplified by PCR [PCR Protocols, Academic Press (1990)] using the prepared cDNA library as the template to obtain a gene fragment encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain.

It can be confirmed that the obtained gene fragment is a DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain, by a method usually used for sequencing a nucleotide, such as the dideoxy method of Sanger et al. [Proc. Natl. Acad. Sci. USA, 74, 5463 (1977)], a nucleotide sequence analyzer such as ABI PRISM 377 DNA Sequencer (manufactured by Applied Biosystems) or the like.

A DNA encoding the enzyme relating to the synthesis of an intracellular sugar nucleotide, GDP-fucose, and/or the enzyme relating to the modification of a sugar chain wherein 1-position of fucose is bound to position of N-acetylglucosamine in the reducing end through α-bond in the complex N-glycoside-linked sugar chain can be obtained by carrying out colony hybridization or plaque hybridization (Molecular Cloning, Second Edition) for the cDNA or cDNA library synthesized from the mRNA contained in the human or non-human animal tissue or cell, using the gene fragment as a DNA probe.

Also, a DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through at-bond in the complex N-glycoside-linked sugar chain can be obtained by carrying out screening by PCR using the primers used for obtaining the gene fragment encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain and using the cDNA or cDNA library synthesized from the mRNA contained in a human or non-human animal tissue or cell as the template.

The nucleotide sequence of the obtained DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain is determined by analyzing the nucleotide sequence from its terminus by a method usually used for sequencing a nucleotide, such as the dideoxy method of Sanger et al. [Proc. Natl. Acad. Sci. USA, 74, 5463 (1977)], a nucleotide sequence analyzer such as ABI PRISM 377 DNA Sequencer (manufactured by Applied Biosystems) or the like.

A gene encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain can also be determined from genes in data bases by searching nucleotide sequence data bases such as GenBank, EMBL, DDBJ and the like using a homology searching program such as BLAST based on the determined cDNA nucleotide sequence.

The nucleotide sequence of the gene obtained by the method encoding the enzyme relating to the synthesis of an intracellular sugar nucleotide, GDP-fucose includes the nucleotide sequence represented by SEQ ID NO:48, 51 or 41. The nucleotide sequence of the gene encoding the enzyme relating to the modification of a sugar chain wherein 1-position of fucose is bound to 6-position of N-acetylglucosamine in the reducing end through α-bond in the complex N-glycoside-linked sugar chain includes the nucleotide sequence represented by SEQ ID NO:1 or 2.

The cDNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain can also be obtained by chemically synthesizing it with a DNA synthesizer such as DNA Synthesizer model 392 manufactured by Perkin Elmer or the like using the phosphoamidite method, based on the determined DNA nucleotide sequence.

As an example of the method for preparing a genomic DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain, the method described below is exemplified.

Preparation Method of Genomic DNA:

As the method for preparing genomic DNA, known methods described in Molecular Cloning, Second Edition; Current Protocols in Molecular Biology; and the like can be mentioned. In addition, a genomic DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain can also be isolated using a kit such as Genomic DNA Library Screening System (manufactured by Genome Systems), Universal GenomeWalker™ Kits (manufactured by CLONTECH) or the like.

The nucleotide sequence of the genoric DNA, obtained by the above method, encoding the enzyme relating to the synthesis of an intracellular sugar nucleotide, GDP-fucose, includes the nucleotide sequence represented by SEQ ID NO:57 or 60. The nucleotide sequence of the genomic DNA encoding the enzyme relating to the modification of a sugar chain wherein 1-position of fucose is bound to 6-position of N-acetylglucosamine in the reducing end through α-bond in the complex N-glycoside-linked sugar chain includes the nucleotide sequence represented by SEQ ID NO:3.

In addition, the host cell of the present invention can also be obtained without using an expression vector, by directly introducing an antisense oligonucleotide or ribozyme into a host cell, which is designed based on the nucleotide sequence encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain.

The antisense oligonucleotide or ribozyme can be prepared in the usual method or using a DNA synthesizer. Specifically, it can be prepared based on the sequence information of an oligonucleotide having a corresponding sequence of continuous 5 to 150 bases, preferably 5 to 60 bases, and more preferably 10 to 40 bases, among nucleotide sequences of a cDNA and a genomic DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain, by synthesizing an oligonucleotide which corresponds to a sequence complementary to the oligonucleotide (antisense oligonucleotide) or a ribozyme comprising the oligonucleotide sequence.

The oligonucleotide includes oligo RNA and derivatives of the oligonucleotide (hereinafter referred to as “oligonucleotide derivatives”).

As the oligonucleotide derivatives, oligonucleotide derivatives in which a phosphodiester bond in the oligonucleotide is converted into a phosphorothioate bond, oligonucleotide derivatives in which a phosphodiester bond in the oligonucleotide is converted into an N3′-P5′ phosphoamidate bond, oligonucleotide derivatives in which ribose and a phosphodiester bond in the oligonucleotide are converted into a peptide-nucleic acid bond, oligonucleotide derivatives in which uracil in the oligonucleotide is substituted with C-5 propynyluracil, oligonucleotide derivatives in which uracil in the oligonucleotide is substituted with C-5 thiazoleuracil, oligonucleotide derivatives in which cytosine in the oligonucleotide is substituted with C-5 propynylcytosine, oligonucleotide derivatives in which cytosine in the oligonucleotide is substituted with phenoxazine-modified cytosine, oligonucleotide derivatives in which ribose in the oligonucleotide is substituted with 2′-O-propylribose, oligonucleotide derivatives in which ribose in the oligonucleotide is substituted with 2′-methoxyethoxyribose [Cell Technology, 16, 1463 (1997)] and the like can be mentioned.

(b) Preparation of the Host Cell for Preparing the Cell of the Present Invention by Homologous Recombination

The host cell for preparing the cell of the present invention can be produced by modifying a target gene on chromosome through a homologous recombination technique, and using a gene encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain as the target gene.

The target gene on the chromosome can be modified by using a method described in Manipulating the Mouse Embryo, A Laboratory Manual, Second Edition, Cold Spring Harbor Laboratory Press (1994) (hereinafter referred to as “Manipulating the Mouse Embiyo, A Laboratory Manual”); Gene Targeting, A Practical Approach, IRL Press at Oxford University Press (1993); Biomanual Series 8, Gene Targeting, Preparation of Mutant Mice using ES Cells, Yodo-sha (1995) (hereinafter referred to as “Preparation of Mutant Mice using ES Cells”); or the like, for example, as follows.

A genomic DNA encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain is prepared.

Based on the nucleotide sequence of the genomic DNA, a target vector is prepared for homologous recombination of a target gene to be modified (e.g., structural gene of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain, or a promoter gene).

The host cell for preparing the cell of the present invention can be produced by introducing the prepared target vector into a host cell and selecting a cell in which homologous recombination occurred between the target gene and target vector.

As the host cell, any cell such as a yeast, an animal cell, an insect cell or a plant cell can be used, so long as it has a gene encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain. Examples include host cells described in the following item 3.

The method for preparing a genomic DNA encoding the enzyme relating to the synthesis of an intracellular sugar nucleotide, GDP-fucose, and/or the enzyme relating to the modification of a sugar chain wherein 1-position of fucose is bound to 6 position of N-acetylglucosamine in the reducing end through at-bond in the complex N-37 glycoside-linked sugar chain includes the methods described in the preparation of genomic DNA in item 1 (1)(a) and the like.

The nucleotide sequence of genomic DNA encoding the enzyme relating to the synthesis of an intracellular sugar nucleotide, GDP-fucose, includes the nucleotide sequence represented by SEQ ID NO-57 or 60. The nucleotide sequence of genomic DNA encoding the enzyme relating to the modification of a sugar chain wherein 1-position of fucose is bound to 6-position of N-acetylglucosamine in the reducing end through α-bond in the complex N-glycoside-linked sugar chain include the nucleotide sequence represented by SEQ ID NO:3.

The target vector for the homologous recombination of the target gene can be prepared in accordance with a method described in Gene Targeting, A Practical Approach, IRL Press at Oxford University Press (1993); Biomanual Series 8, Gene Targeting, Preparation of Mutant Mice using ES Cells, Yodo-sha (1995); or the like. The target vector can be used as either a replacement type or an insertion type.

For introducing the target vector into various host cells, the methods for introducing recombinant vectors suitable for various host cells, which are described in the following item 3, can be used.

The method for efficiently selecting a homologous recombinant includes a method such as the positive selection, promoter selection, negative selection or polyA selection described in Gene Targeting, A Practical Approach, IRL Press at Oxford University Press (1993); Biomanual Series 8, Gene Targeting, Preparation of Mutant Mice using ES Cells, Yodo-sha (1995); or the like. The method for selecting the homologous recombinant of interest from the selected cell lines includes the Southern hybridization method for genomic DNA (Molecular Cloning, Second Edition), PCR [PCR Protocols, Academic Press (1990)], and the like.

(c) Preparation of the Host Cell for Preparing the Cell of the Present Invention by RDO Method

The host cell for preparing the cell of the present invention can be prepared by an RDO (RNA-DNA oligonucleotide) method by targeting a gene encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain, for example, as follows.

A cDNA or a genomic DNA encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain is prepared.

The nucleotide sequence of the prepared cDNA or genomic DNA is determined.

Based on the determined DNA sequence, an appropriate length of an RDO construct comprising a DNA which encodes the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain. The designed RDO construct can further comprise a part of non translation region or a part of an intron.

The host cell of the present invention can be obtained by introducing the synthesized RDO into a host cell and then selecting a transformant in which a mutation occurred in the target enzyme, that is, the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through ol-bond in the complex N-glycoside-linked sugar chain.

As the host cell, any cell such as a yeast, an animal cell, an insect cell or a plant cell can be used, so long as it has a gene encoding the target enzyme relating to the synthesis of an intracellular sugar nucleotide, GDP-fucose, and/or the target enzyme relating to the modification of a sugar chain wherein 1-position of fucose is bound to 6-position of N-acetylglucosamine in the reducing end through α-bond in the complex N-glycoside-linked sugar chain. Examples include host cells described in the following item 3.

The method for introducing RDO into various host cells includes the methods for introducing recombinant vectors suitable for various host cells, which are described in the following item 3.

The method for preparing cDNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain include the methods described in the preparation method of DNA in item 1(1)(a) and the like.

The method for preparing a genomic DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain include the methods in preparation of genomic DNA described in item 1(1)(a) and the like.

The nucleotide sequence of the DNA can be determined by digesting it with appropriate restriction enzymes, cloning the DNA fragments into a plasmid such as pBluescript SK(−) (manufactured by Stratagene) or the like, subjecting the clones to the reaction generally used as a method for analyzing a nucleotide sequence such as the dideoxy method [Proc. Natl. Acad Sci USA, 74, 5463 (1977)] of Sanger et al. or the like, and then analyzing the clones using an automatic nucleotide sequence analyzer such as A.L.F. DNA Sequencer (manufactured by Pharmacia) or the like.

The RDO can be prepared by a usual method or using a DNA synthesizer.

The method for selecting a cell in which a mutation occurred, by introducing the RDO into the host cell, in the gene encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain includes the methods for directly detecting mutations in chromosomal genes described in Molecular Cloning, Second Edition, Current Protocols in Molecular Biology and the like.

Also, as the method, the method described in item 1(1)(a) for selecting a transformant through the evaluation of the activity of the introduced enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain; the method for selecting a transformant based on the sugar chain structure of a glycoprotein on the cell membrane which will be described later in item 1(5); and the method for selecting a transformant based on the sugar chain structure of the produced antibody molecule which will be described later in item 4 or 5, and the like can be used.

The construct of the RDO can be designed in accordance with the methods described in Science, 273, 1386 (1996); Nature Medicine, 4, 285 (1998); Hepatoloy, 25, 1462 (1997); Gene Therapy, 5, 1960 (1999); J. Mol. Med., 75, 829 (1997); Proc. Natl. Acad Sci. USA, 96, 8774 (1999); Proc. Natl. Acad. Sci. USA, 96, 8768 (1999); Nuc. Acids. Res., 7, 1323 (1999); Invent Dematol., 111, 1172 (1998); Nature Biotech., 16, 1343 (1998); Nature Biotech., 18, 43 (2000); Nature Biotech., A, 555 (2000); and the like.

(d) Preparation of the Host Cell for Preparing the Cell of the Present Invention by the RNAi Method

The host cell for preparing the cell of the present invention can be prepared by the RNAi (RNA interference) method by targeting a gene of an enzyme relating to the synthesis of an intracellular sugar nucleotide, GDP-fucose, and/or of an enzyme relating to the modification of a sugar chain wherein 1-position of fucose is bound to 6-position of N-acetylglucosamine in the reducing end through α-bond in the complex N-glycoside-linked sugar chain, for example, as follows.

A cDNA encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain is prepared.

The nucleotide sequence of the prepared cDNA is determined.

Based on the determined DNA sequence, an appropriate length of an RNAi gene construct comprising a part of DNA encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain is designed. The designed construct can ether comprise a part of its non-translation region.

In order to express the RNAi gene in a cell, a recombinant vector is prepared by inserting a fragment or full length of the prepared DNA into downstream of the promoter of an appropriate expression vector.

A transformant is obtained by introducing the recombinant vector into a host cell suitable for the expression vector.

The host cell for preparing the cell of the present invention can be obtained by selecting a transformant based on the activity of the enzyme relating to the synthesis of an intracellular sugar nucleotide, GDP-fucose, and/or of the enzyme relating to the modification of a sugar chain wherein 1-position of fucose is bound to 6-position of N-acetylglucosamine in the reducing end through Cα-bond in the complex N-glycoside-linked sugar chain, or based on the sugar chain structure of a glycoprotein on the cell membrane or of the produced antibody molecule.

As the host cell, any cell such as a yeast, an animal cell, an insect cell or a plant cell can be used, so long as it has a gene encoding the target enzyme relating to the synthesis of an intracellular sugar nucleotide, GDP-fucose, and/or the target enzyme relating to the modification of a sugar chain wherein 1-position of fucose is bound to 6 position of N-acetylglucosamine in the reducing end through α-bond in the complex N-glycoside-linked sugar chain. Examples include host cells described in the following item 3.

As the expression vector, a vector which is autonomously replicable in the host cell or can be integrated into the chromosome and comprises a promoter at such a position that the designed RNAi gene can be transferred is used. Examples include expression vectors described in the following item 3.

As the method for introducing a gene into various host cells, the methods for introducing recombinant vectors suitable for various host cells, which are described in the following item 3, can be used.

The method for selecting a transformant based on the activity of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain includes the methods described in item 1(1)(a).

The method for selecting a transformant based on the sugar chain structure of a glycoprotein on the cell membrane includes the methods which will be described later in item 1(5). The method for selecting a transformant based on the sugar chain structure of a produced antibody molecule includes the methods described in the following item 4 or 5.

The method for preparing cDNA encoding the enzyme relating to the synthesis of an intracellular sugar nucleotide, GDP-fucose, or the enzyme relating to the modification of a sugar chain wherein 1-position of fucose is bound to 6-position of N-acetylglucosamine in the reducing end through α-bond in the complex N-glycoside-linked sugar chain includes the methods described in the preparation method of DNA in item 1(1)(a) and the like.

In addition, the host cell for preparing the cell of the present invention can also be obtained without using an expression vector, by directly introducing an RNAi gene designed based on the nucleotide sequence encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain.

The RNAi gene can be prepared in the usual method or using a DNA synthesizer.

The RNAi gene construct can be designed in accordance with the methods described in Nature, 391, 806 (1998); Proc. Natl. Acad. Sci. USA, 95, 15502 (1998); Nature, 95, 854.(1998); Proc. Natl. Acad. Sci. USA, 96, 5049 (1999); Cell, 95, 1017 (1998); Proc. Natl. Acad. Sci. USA, 96, 1451 (1999); Proc. Natl. Acad. Sci. USA, 95, 13959 (1998); Nature Cell Biol., 2, 70 (2000); and the like.

(e) Preparation of the Host Cell for Preparing the Cell of the Present Invention by the Method Using Transposon

The host cell for preparing the cell of the present invention can be prepared by inducing mutation using a transposon system described in Nature Genet., 25, 35 (2000) or the like, and then by selecting a mutant based on the activity of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain, or based on the sugar chain structure of a glycoprotein of a produced antibody molecule or on the cell membrane.

The transposon system is a system in which a mutation is induced by randomly inserting an exogenous gene into chromosome, wherein an exogenous gene interposed between transposons is generally used as a vector for inducing a mutation, and a transposase expression vector for randomly inserting the gene into chromosome is introduced into the cell at the same time.

Any transposase can be used, so long as it is suitable for the sequence of the transposon to be used.

As the exogenous gene, any gene can be used, so long as it can induce a mutation in the DNA of a host cell.

As the host cell, any cell such as a yeast, an animal cell, an insect cell or a plant cell can be used, so long as it has a gene encoding the target enzyme relating to the synthesis of an intracellular sugar nucleotide, GDP-fucose, and/or of the target enzyme relating to the modification of a sugar chain wherein 1-position of fucose is bound to 6-position of N-acetylglucosamine in the reducing end through α-bond in the complex N-glycoside-linked sugar chain. Examples include host cells described in the following item 3. For introducing the gene into various host cells, the methods for introducing recombinant vectors suitable for various host cells, which are described in the following item 3, can be used.

The method for selecting a mutant based on the activity of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain includes the methods described in item 1(1)(a).

The method for selecting a mutant based on the sugar chain structure of a glycoprotein on the cell membrane includes the methods described in the following item 1(5). The method for selecting a mutant based on the sugar chain structure of a produced antibody molecule includes the methods described in the following item 4 or 5,

(2) Method for Introducing a Dominant Negative Mutant of a Gene Encoding an Enzyme

The host cell for preparing the cell of the present invention can be prepared by targeting a gene encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain, and using a technique for introducing a dominant negative mutant of the enzyme. The enzyme relating to the synthesis of an intracellular sugar nucleotide, GDP-fucose, includes GMD, Fx, GFPP, fucokinase and the like. The enzyme relating to the modification of a sugar chain wherein 1-position of fucose is bound to 6-position of N-acetylglucosamine in the reducing end through α-bond in the complex N-glycoside-linked sugar chain includes α1,6-fucosyltransferase, α-L-fucosidase and the like.

The enzymes catalyze specific reactions having substrate specificity, and dominant negative mutants of a gene encoding the enzymes can be prepared by disrupting the active center of the enzymes which catalyze the catalytic activity having substrate specificity. The method for preparing a dominant negative mutant is specifically described as follows with reference to GMD among the target enzymes.

As a result of the analysis of the three-dimensional structure of E. coli-derived GMD, it has been found that 4 amino acids (threonine at position 133, glutamic acid at position 135, tyrosine at position 157 and lysine at position 161) have an important function on the enzyme activity (Stacture, 8, 2, 2000). That is, when mutants were prepared by substituting the 4 amino acids with other different amino acids based on the three-dimensional structure information, the enzyme activity of all of the mutants was significantly decreased. On the other hand, changes in the ability of mutant GMD to bind to GMD coenzyme NADP or its substrate GDP-mannose were hardly observed. Accordingly, a dominant negative mutant can be prepared by substituting the 4 amino acids which control the enzyme activity of GMD. For example, in GMD (SEQ ID NO:41) derived from CHO cell, a dominant negative mutant can be prepared by substituting threonine at position 155, glutamic acid at position 157, tyrosine at position 179 and lysine at position 183 with other amino acids, by comparing the homology and predicting the three-dimensional structure using the amino acid sequence information based on the results of the E. coli-derived GMD. Such a gene into which amino acid substitution is introduced can be prepared by the site-directed mutagenesis described in Molecular Cloning, Second Edition, Current Protocols in Molecular Biology or the like.

The host cell for preparing the cell of the present invention can be prepared in accordance with the method described in Molecular Cloning, Second Edition, Current Protocols in Molecular Biology or the like, using the prepared dominant negative mutant gene of the target enzyme, for example, as follows.

A gene encoding a dominant negative mutant (hereinafter referred to as “dominant negative mutant gene”) of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain is prepared.

Based on the prepared full length DNA of dominant negative mutant gene, a DNA fragment of an appropriate length containing a moiety encoding the protein is prepared, if necessary.

A recombinant vector is produced by inserting the DNA fragment or full length DNA into downstream of the promoter of an appropriate expression vector.

A transformant is obtained by introducing the recombinant vector into a host cell suitable for the expression vector, The host cell for preparing the cell of the present invention can be prepared by selecting a transformant based on the activity of the-enzyme relating to the synthesis of an intracellular sugar nucleotide, GDP-fucose, and/or the activity of the enzyme relating to the modification of a sugar chain wherein 1-position of fucose is bound to 6-position of N-acetylglucosamine in the reducing end through α-bond in the complex N-glycoside-linked sugar chain, or the sugar chain structure of a glycoprotein of a produced antibody molecule or on the cell membrane.

As the host cell, any cell such as a yeast, an animal cell, an insect cell or a plant cell can be used, so long as it has a gene encoding the target enzyme relating to the synthesis of an intracellular sugar nucleotide, GDP-fucose, and/or the target enzyme relating to the modification of a sugar chain wherein 1-position of fucose is bound to 6-position of N-acetylglucosamine in the reducing end through α-bond in the complex N-glycoside-linked sugar chain. Examples include the host cells which will be described later in the following item 3.

As the expression vector, a vector which is autonomously replicable in the host cell or can be integrated into the chromosome and comprises a promoter at a position where transcription of the DNA encoding the dominant negative mutant of interest can be effected is used. Examples include expression vectors described in the following item 3.

For introducing the gene into various host cells, the methods for introducing recombinant vectors suitable for various host cells, which are described in the following item 3, can be used.

The method for selecting a transformant based on the activity of the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain includes the methods described in item 1(1)(a).

The method for selecting a transformant based on the sugar chain structure of a glycoprotein on the cell membrane includes the methods described in item 1(5). The method for selecting a transformant based on the sugar chain structure of a produced antibody molecule includes methods described in the following item 4 or 5.

(3) Method for Introducing a Mutation into an Enzyme

The host cell for preparing the cell of the present invention can be prepared by introducing a mutation into a gene encoding an enzyme relating to the synthesis of an intracellular sugar nucleotide, GDP-fucose, or an enzyme relating to the modification of a sugar chain wherein 1-position of fucose is bound to 6-position of N-acetylglucosamine in the reducing end through α-bond in the complex N-glycoside-linked sugar chain, and then by selecting a cell line of interest in which the mutation occurred in the enzyme.

The enzyme relating to the synthesis of an intracellular sugar nucleotide, GDP-fucose, includes GMD, Fx, GFPP, fucokinase and the like. The enzyme relating to the modification of a sugar chain wherein 1-position of fucose is bound to 6-position of N-acetylglucosamine in the reducing end through α-bond in the complex N-glycoside-linked sugar chain includes α1,6-fucosyltransferase, α-L-fucosidase and the like.

The method includes 1) a method in which a desired cell line is selected from mutants obtained by a mutation-inducing treatment of a parent cell line with a mutagen or spontaneously generated mutants, based on the activity of an enzyme relating to the synthesis of an intracellular sugar nucleotide, GDP-fucose, and/or the activity of an enzyme relating to the modification of a sugar chain wherein 1-position of fucose is bound to 6-position of N-acetylgucosamine in the reducing end through α-bond in the complex N-glycoside-linked sugar chain, 2) a method in which a desired cell line is selected from mutants obtained by a mutation-inducing treatment of a parent cell line with a mutagen or spontaneously generated mutants, based on the sugar chain structure of a produced antibody molecule, and 3) a method in which a desired cell line is selected from mutants obtained by a mutation-inducing treatment of a parent cell line with a mutagen or spontaneously generated mutants, based on the sugar chain structure of a glycoprotein on the cell membrane.

As the mutation-inducing treatment, any treatment can be used, so long as it can induce a point mutation or a deletion or frame shift mutation in the DNA of cells of the parent cell line.

Examples include treatment with ethyl nitrosourea, nitrosoguanidine, benzopyrene or an acridine pigment and treatment with radiation. Also, various alkylating agents and carcinogens can be used as mutagens. The method for allowing a mutagen to act upon cells includes the methods described in Tissue Culture Techniques, 3rd edition (Asakura Shoten), edited by Japanese Tissue Culture Association (1996), Nature Genet., 24, 314 (2000) and the like.

The spontaneously generated mutant includes mutants which are spontaneously formed by continuing subculture under general cell culture conditions without applying special mutation-inducing treatment.

The method for measuring the activity of the enzyme relating to the synthesis of an intracellular sugar nucleotide, GDP-fucose, and/or the activity of the enzyme relating to the modification of a sugar chain wherein 1-position of fucose is bound to 6-position of N-acetylglucosamine in the reducing end through α-bond in the complex N-glycoside-linked sugar chain includes the methods described in item 1(1)(a). The method for discriminating the sugar chain structure of a prepared antibody molecule includes methods described in the following item 4 or 5. The method for discriminating the sugar chain structure of a glycoprotein on the cell membrane includes the methods described in item 1(5).

(4) Method for Inhibiting Transcription and/or Translation of a Gene Encoding an Enzyme

The host cell of the present invention can be prepared by inhibiting transcription and/or translation of a target gene through a method such as the antisense RNA/DNA technique [Bioscience and Industry, 50, 322 (1992); Chemistry, 46, 681 (1991); Biotechnology, 9, 358 (1992); Trends in Biotechnology, 10, 87 (1992); Trends in Biotechnology, 10, 152 (1992); Cell Engineering, 16, 1463 (1997)], the triple helix technique [Trends in Biotechnology, 10, 132 (1992)] or the like, using a gene encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through α-bond in the complex N-glycoside-linked sugar chain, as the target.

The enzyme relating to the synthesis of an intracellular sugar nucleotide, GDP-fucose includes GMD, Fx, GFPP, fucokinase and the like. The enzyme relating to the modification of a sugar chain wherein 1-position of fucose is bound to 6-position of N-acetylglucosamine in the reducing end through α-bond in the complex N-glycoside-linked sugar chain includes α1,6-fucosyltransferase, α-L-fucosidase and the like.

(5) Method for Selecting a Cell Line Resistant to a Lectin which Recognizes a Sugar Chain Structure in which 1-position of Fucose is Bound to 6-position of N-acetylglucosamine in the Reducing End Through α-bond in the N-glycoside-linked Sugar Chain

The host cell for preparing the cell of the present invention can be prepared by using a method for selecting a cell line resistant to a lectin which recognizes a sugar chain structure in which 1-position of fucose is bound to 6-position of N-acerylglucosamine in the reducing end through o-bond in the N-glycoside-linked sugar chain.

The method for selecting a cell line resistant to a lectin which recognizes a sugar chain structure in which 1-position of fucose is bound to 6-position of N-acetylglucosamine in the reducing end through α-bond in the N-glycoside-linked sugar chain includes the methods using lectin described in Somatic Cell Mol. Genet., 12, 51 (1986) and the like.

As the lectin, any lectin can be used, so long as it is a lectin which recognizes a sugar chain structure in which 1-position of fucose is bound to 6-position of N-acetylglucosamine in the reducing end through α-bond in the N-glycoside-linked sugar chain. Examples include a Lens culinaris lectin LCA (lentil agglutinin derived from Lens culinaris), a pea lectin PSA (pea lectin derived from Pisum sativum), a broad bean lectin VFA (agglutinin derived from Vicia faba), an Aleuia aurantia lectin AAL (lectin derived from Aleuria aurantia) and the like.

Specifically, the cell line of the present invention resistant to a lectin which recognizes a sugar chain structure in which 1-position of fucose is bound to 6-position of N-acetylglucosamine in the reducing end through α-bond in the N-glycoside-linked sugar chain can be selected by culturing cells for 1 day to 2 weeks, preferably from 1 day to 1 week, using a medium comprising the lectin at a concentration of 1 μg/ml to 1 mg/ml, subculturing surviving cells or picking up a colony and transferring it into a culture vessel, and subsequently continuing the culturing using the lectin-containing medium.

2. Preparation of a Transgenic Non-Human Animal or Plant or the Progenies Thereof of the Present Invention

The transgenic non-human animal or plant or the progenies thereof in which a genome gene is modified in such a manner that the activity of an enzyme relating to the modification of a sugar chain of an antibody molecule can be controlled can be prepared from the embryonic stem cell, the fertilized egg cell or the plant callus cell of the present invention prepared by the above item 1 using a gene encoding an enzyme relating to the synthesis of an intracellular sugar nucleotide, GDP-fucose, and/or an enzyme relating to the modification of a sugar chain wherein 1-position of fucose is bound to 6-position of N-acetylglucosamine in the reducing end through at-bond in the complex N-glycoside-linked sugar chain, as the target, for example, as follows.

In a transgenic non-human animal, the embryonic stem cell of the present invention in which the activity of the enzyme relating to the synthesis of an intracellular sugar nucleotide, GDP-fucose, and/or the activity of the enzyme relating to the modification of a sugar chain wherein 1-position of fucose is bound to 6-position of N-acetylglucosamine in the reducing end through α-bond in the complex N-glycoside-linked sugar chain is controlled can be prepared by the method described in item 1 to an embryonic stem cell of the intended non-human animal such as cattle, sheep, goat, pig, horse, mouse, rat, fowl, monkey, rabbit or the like.

Specifically, a mutant clone is prepared in which a gene encoding the enzyme relating to the synthesis of an intracellular sugar nucleotide, GDP-fucose, and/or the enzyme relating to the modification of a sugar chain wherein 1-position of fucose is bound to 6-position of N-acetylglucosamine in the reducing end through α-bond in the complex N-glycoside-linked sugar chain is inactivated or substituted with any sequence, by a known homologous recombination technique [e.g., Nature, 326, 6110, 295 (1987); Cell, 51, 3, 503 (1987); or the like]. Using the prepared embryonic stem cell (e.g., the mutant clone), a chimeric individual comprising the embryonic stem cell clone and a normal cell can be prepared by an injection chimera method into blastocyst of fertilized egg of an animal or by an aggregation chimera method. The chimeric individual is crossed with a normal individual, so that a transgenic non-human animal in which the activity of the enzyme relating to the synthesis of an intracellular sugar nucleotide, GDP-fucose, and/or the activity of the enzyme relating to the modification of a sugar chain wherein 1-position of fucose is bound to 6-position of N-acetylglucosamine in the reducing end through α-bond in the complex N-glycoside-linked sugar chain is decreased or deleted in the whole body cells can be obtained.

Also, a fertilized egg cell of the present invention in which the activity of an enzyme relating to the synthesis of an intracellular sugar nucleotide, GDP-fucose, and/or the activity of an enzyme relating to the modification of a sugar chain wherein 1-position of fucose is bound to 6-position of N-acetylglucosamine in the reducing end through α-bond in the complex N-glycoside-linked sugar chain is decreased or deleted can be prepared by applying the method similar to that in item 1 to fertilized egg of a non-human animal of interest such as cattle, sheep, goat, pig, horse, mouse, rat, fowl, monkey, rabbit or the like.

A transgenic non-human animal in which the activity of an enzyme relating to the synthesis of an intracellular sugar nucleotide, GDP-fucose, and/or the activity of an enzyme relating to the modification of a sugar chain wherein 1-position of fucose is bound to 6-position of N-acetylglucosamine in the reducing end through CL-bond in the complex N-glycoside-linked sugar chain is decreased can be prepared by transplanting the prepared fertilized egg cell into the oviduct or uterus of a pseudopregnant female using the embryo transplantation method described in Manipulating Mouse Embryo, Second Edition or the like, followed by childbirth by the animal.

In a transgenic plant, the callus of the present invention in which the activity of an enzyme relating to the synthesis of an intracellular sugar nucleotide, GDP-fucose, and/or the activity of an enzyme relating to the modification of a sugar chain wherein 1 position of fucose is bound to 6-position of N-acetylglucosamine in the reducing end through α-bond in the complex N-glycoside-linked sugar chain is decreased or deleted can be prepared by applying the method similar to that in item 1 to a callus or cell of the plant of interest.

A transgenic plant in which the activity of an enzyme relating to the synthesis of an intracellular sugar nucleotide, GDP-fucose, and/or the activity of an enzyme relating to the modification of a sugar chain wherein 1-position of fucose is bound to 6-position of N-acetylglucosamine in the reducing end through α-bond in the complex N-glycoside-linked sugar chain is decreased can be prepared by culturing the prepared callus using a medium comprising auxin and cytokinin to redifferentiate it in accordance with a known method [Tissue Culture, 20 (1994); Tissue Culture, 21 (1995); Trends in Biotechnology, 15, 45 (1997)]3

3. Process for Producing the Antibody Composition

The antibody composition can be obtained by expressing it in a host cell using the methods described in Molecular Cloning, Second Edition; Current Protocols in Molecular Biology, Antibodies, A Laboratory Manual, Cold Spring Harbor Laboratory, 1988 (hereinafter referred also to as “Antibodies”); Monoclonal Antibodies: Principles and Practice, Third Edition, Acad. Press, 1993 (hereinafter referred also to as “Monoclonal Antibodies”); and Antibody Engineering, A Practical Approach, IRL Press at Oxford University Press (hereinafter referred also to as “Antibody Engineering”), for, example, as follows.

A full length cDNA encoding the anti-CD20 antibody molecule of the present invention is prepared, and an appropriate length of a DNA fragment comprising a region encoding the antibody molecule is prepared.

A recombinant vector is prepared by inserting the DNA fragment or the full length cDNA into downstream of the promoter of an appropriate expression vector.

A transformant which produces the antibody molecule can be obtained by introducing the recombinant vector into a host cell suitable for the expression vector.

As the host cell, any of a yeast, an animal cell, an insect cell a plant cell or the like can be used, so long as it can express the gene of interest.

As the host cell, a cell into which an enzyme relating to the modification of an N-glycoside-linked sugar chain which binds to the Fc region of the antibody molecule, i.e., an enzyme relating to the synthesis of an intracellular sugar nucleotide, GDP-fucose and/or the activity of an enzyme relating to the modification of a sugar chain in which 1-position of fucose is bound to 6-position of N-acetylglucosamine in the reducing end through α-bond in the complex N-glycoside-linked sugar chain is decreased or deleted or a cell obtained by various artificial techniques described in item 1 can also be used.

As the expression vector, a vector which is autonomously replicable in the host cell or can be integrated into the chromosome and comprises a promoter at such a position that the DNA encoding the antibody molecule of interest can be transferred is used.

The cDNA can be prepared from a human or non-human tissue or cell using, e.g., a probe primer specific for the antibody molecule of interest, in accordance with the methods described in the preparation method of DNA in item 1(1)(a).

When a yeast is used as the host cell, the expression vector includes YEP13 (ATCC 37115), YEp24 (ATCC 37051), YCp50 (ATCC 37419) and the like.

Any promoter can be used, so long as it can function in yeast. Examples include a promoter of a gene of the glycolytic pathway such as a hexose kinase gene, PH05 promoter, PGK promoter, GAP promoter, ADH promoter, gal 1 promoter, gal 10 promoter, heat shock protein promoter, ME α1 promoter, CUP 1 promoter and the like.

The host cell includes microorganisms belonging to the genus Saccharomyces, the genus Schizosaccharomyces, the genus Kluyveromyces, the genus Trichosporon, the genus Schwanniomyces and the like, such as Saccharomyces cerevisiae, Schizosaccharomyces pombe, Kluyveromyces lactis, Trichosporon pulularis and Schwanniomyces alluvius.

As the method for introducing the recombinant vector, any method can be used, so long as it can introduce DNA into yeast. Examples include electroporation [Methods in Enzymology, 194, 182 (1990)], the spheroplast method [Proc. Natl. Acad. Sci. USA, 94, 1929 (1978)], the lithium acetate method [J. Bacteriol., 153, 163 (1983)], the method described in Proc. Natl. Acad. Sci. USA, 75, 1929 (1978) and the like.

When an animal cell is used as the host, the expression vector includes pcDNAI, pcDM8 (available from Funakoshi), pAGE107 [Japanese Published Examined Patent Application No. 22979/91; Cytotechnology, 3, 133 (1990)], pAS3-3 (Japanese Published Examined Patent Application No. 227075/90), pCDM8 [Nature, 329, 840 (1987)], pcDNAI/Amp (manufactured by Invitrogen), pREP4 (manufactured by Invitrogen), pAGE103 [J. Biochemistry, 101, 1307 (1987)], pAGE210 and the like.

Any promoter can be used, so long as it can function in an animal cell. Examples include a promoter of IE (immediate early) gene of cytomegalovirus (CMV), an early promoter of SV40, a promoter of retrovirus, a promoter of metallothionein, a heat shock promoter, an SRα promoter and the like. Also, an enhancer of the IE gene of human CMV may be used together with the promoter.

The host cell includes a human cell such as Namalwa cell, a monkey cell such as COS cell, a Chinese hamster cell such as CHO cell or HBT5637 (Japanese -Published Examined Patent Application No. 299/88), a rat myeloma cell, a mouse myeloma cell, a cell derived from syrian hamster kidney, an embryonic stem cell, a fertilized egg cell and the like.

As the method for introducing the recombinant vector, any method can be used, so long as it can introduce DNA into an animal cell. Examples include electroporation [Cytotechnology, 3, 133 (1990)], the calcium phosphate method (Japanese Published Examined Patent Application No. 227075/90), the lipofection method [Proc. Natl. Acad. Sci. USA, 84, 7413 (1987)], the injection method [Manipulating the Mouse Embryo, A Laboratory Manual], the method using particle gun (gene gun) (Japanese Patent No. 2606856, Japanese Patent No. 2517813), the DEAE-dextran method [Biomanual Series 4-Gene Transfer and Expression Analysis (Yodo-sha), edited by Takashi Yokota and Kenichi Arai (1994)], the virus vector method [Manipulating Mouse Embryo, Second Edition] and the like.

When an insect cell is used as the host, the protein can be expressed by the method described in Current Protocols in Molecular Biology, Baculovirus Expression Vectors, A Laboratory Manual, W. H. Freeman and Company, New York (1992), Biotechnology, 6, 47 (1988) or the like.

That is, the protein can be expressed by co-introducing a recombinant gene-introducing vector and a baculovirus into an insect cell to obtain a recombinant virus in an insect cell culture supernatant and then infecting the insect cell with the recombinant virus.

The gene introducing vector used in the method includes pVL1392, pVL1393, pBlueBacm (all manufactured by Invitrogen) and the like.

The baculovirus includes Autographa californica nuclear polyhedrosis virus infected with an insect of the family Barathra.

The insect cell includes Spodoptera frugiperda ovarian SP9 and Sf21 [Current Protocols in Molecular Biology, Baculovirus Expression Vectors, A Laboratory Manual, W. H. Freeman and Company, New York (1992)], a Trichoplusia ni ovarian High 5 (manufactured by Invitrogen) and the like.

The method for the simultaneously introducing the recombinant gene-introducing vector and the baculovirus for preparing the recombinant virus includes the calcium phosphate method (Japanese Published Examined Patent Application No. 227075/90), the lipofection method [Proc. Natl. Acad Sci. USA, 84, 7413 (1987)] and the like.

When a plant cell is used as the host cell, the expression vector include Ti plasmid, tobacco mosaic virus and the like.

As the promoter, any promoter can be used, so long as it can function in a plant cell. Examples include cauliflower mosaic virus (CaMV) 35S promoter, rice actin 1 promoter and the like.

The host cell includes plant cells of tobacco, potato, tomato, carrot soybean, rape, alfalfa, rice, wheat, barley, and the like.

As the method for introducing the recombinant vector, any method can be used, so long as it can introduce DNA into a plant cell. Examples include a method using Agrobacterium (Japanese Published Examined Patent Application No. 140885/84, Japanese Published Examined Patent Application No. 70080/85, WO 94/00977), electroporation (Japanese Published Examined Patent Application No. 251887/85), a method using a particle gun (gene gun) (Japanese Patent No. 2606856, Japanese Patent No. 2517813) and the like.

As the method for expressing a gene, secretion production, expression of a fusion protein of the Fc region with other protein and the like can be carried out in accordance with the method described in Molecular Cloning, Second Edition or the like, in addition to the direct expression.

When a gene is expressed by a bacterium, a yeast, an animal cell, an insect cell or a plant cell into which a gene relating to the synthesis of a sugar chain is introduced, an antibody molecule to which a sugar or a sugar chain is added by the introduced gene can be obtained.

An antibody composition can be obtained by culturing the obtained transformant in a medium to form and accumulate the antibody molecule in the culture and then recovering it from the culture. The method for culturing the transformant using a medium can be carried out in accordance with a general method which is used for the culturing of host cells.

As the medium for culturing a transformant obtained by using a prokaryote such as Escherichia coli or a eukaryote such as yeast as the host, the medium may be either a natural medium or a synthetic medium, so long as it comprises materials such as a carbon source, a nitrogen source, an inorganic salt and the like which can be assimilated by the organism and culturing of the transformant can be efficiently carried out.

As the carbon source, those which can be assimilated by the organism can be used. Examples include carbohydrates such as glucose, fructose, sucrose, molasses containing them, starch, and starch hydrolysate; organic acids such as acetic acid and propionic acid; alcohols such as ethanol and propanol; and the like.

The nitrogen source includes ammonia; ammonium salts of inorganic acid or organic acid such as ammonium chloride, ammonium sulfate, ammonium acetate and ammonium phosphate; other nitrogen-containing compounds; peptone; meat extract; yeast extract; corn steep liquor, casein hydrolysate; soybean meal; soybean meal hydrolysate; various fermented cells and hydrolysates thereof; and the like.

The inorganic material includes potassium dihydrogen phosphate, dipotassium hydrogen phosphate, magnesium phosphate, magnesium sulfate, sodium chloride, ferrous sulfate, manganese sulfate, copper sulfate, calcium carbonate, and the like.

The culturing is carried out generally under aerobic conditions such as shaking culture or submerged-aeration stirring culture. The culturing temperature is preferably 15 to 40° C., and the culturing time is generally 16 hours to 7 days. During the culturing, the pH is maintained at 3.0 to 9.0. The pH is adjusted using inorganic or organic acid, an alkali solution, urea, calcium carbonate, ammonia or the like.

Also, if necessary, an antibiotic such as ampicillin or tetracycline may be added to the medium during the culturing.

When a microorganism transformed with a recombinant vector obtained by using an inducible promoter as the promoter is cultured, an inducer may be added to the medium, if necessary. For example, when a microorganism transformed with a recombinant vector obtained by using lac promoter is cultured, isopropyl-β-D-thiogalactopyranoside may be added to the medium, and when a microorganism transformed with a recombinant vector obtained by using trip promoter is cultured, indoleacrylic acid may be added to the medium.

When a transformant obtained by using an animal cell as the host is cultured, examples of the medium include generally used RPMI 1640 medium [The Journal of the American Medical Association, 199, 519 (1967)], Eagle's NEM medium [Science, 122, 501 (1952)], Dulbecco's modified MEM medium [Virology, 8, 396 (1959)], 199 medium [Proceeding of the Society for the Biological Medicine, 73, 1 (1950)] and Whitten's medium [Developmental Engineering Experimentation Manual-Preparation of Transgenic Mice (Kodan-sha), edited by M. Katshuki (1987)], wherein the media are added to fetal calf serum.

The culturing is carried out generally at a pH of 6 to 8 and 30 to 40° C. for 1 to 7 days in the presence of 5% CO₂.

If necessary, an antibiotic such as kanamycin or penicillin may be added to the medium during the culturing.

The medium for culturing of a transformant obtained by using an insect cell as the host includes usually used TNM-FH medium (manufactured by Pharmingen), Sf-900 II SFM medium (manufactured by Life Technologies), ExCell 400 and ExCell 405 (both manufactured by JRH Biosciences), Grace's Insect Medium [Nature, 195, 788 (1962)] and the like.

The culturing is carried out generally at a medium pH of 6 to 7 and 25 to 30° C. for 1 to 5 days.

If necessary, antibiotics such as gentamicin may be added to the medium during the culturing.

A transformant obtained by using a plant cell as the host can be cultured as a cell or by differentiating it into a plant cell or organ. The medium for culturing the transformant includes generally used Murashige and Skoog (MS) medium and White medium, wherein the media are added to a plant hormone such as auxin or cytokinin.

The culturing is carried out generally at a pH of 5 to 9 and 20 to 40° C. for 3 to 60 days.

If necessary, an antibiotic such as kanamycin, hygromycin or the like may be added to the medium during the culturing.

Thus, an antibody composition can be produced by culturing a transformant derived from a microorganism, an animal cell or a plant cell, which comprises a recombinant vector into which a DNA encoding an antibody molecule is inserted, in accordance with a general culturing method, to thereby form and accumulate the antibody composition, and then recovering the antibody composition from the culture.

As the method for expressing the gene, secretion production, expression of a fusion protein and the like can be carried out in accordance with the method described in Molecular Cloning, Second Edition, in addition to the direct expression.

The method for producing an antibody composition includes a method of intracellular expression in a host cell, a method of extracellular secretion from a host cell, and a method of production on a host cell membrane outer envelope. The method can be selected by changing the host cell used or the structure of the antibody composition produced.

When the antibody composition of the present invention is produced in a host cell or on a host cell membrane outer envelope, it can be positively secreted extracellularly in accordance with the method of Paulson et al. [J. Biol. Chem., 264, 17619 (1989)], the method of Lowe et al. [Proc. Natl. Acad. Sci. USA, 86, 8227 (1989), Genes Develop., 4, 1288 (1990)], the methods described in Japanese Published Examined Patent Application No. 336963/93 and Japanese Published Examined Patent Application No. 823021/94 and the like.

That is, an antibody molecule of interest can be positively secreted extracellularly from a host cell by inserting a DNA encoding the antibody molecule and a DNA encoding a signal peptide suitable for the expression of the antibody molecule into an expression vector using a recombinant DNA technique, introducing the expression vector into the host cell and then expressing the antibody molecule.

Also, its production amount can be increased in accordance with the method described in Japanese Published Examined Patent Application No. 227075/90 using a gene amplification system using a dihydrofolate reductase gene.

In addition, the antibody composition can also be produced by using a gene-introduced animal individual (transgenic non-human animal) or a plant individual (transgenic plant) which is constructed by the redifferentiation of an animal or plant cell into which the gene is introduced.

When the transformant is an animal individual or a plant individual, an antibody composition can be produced in accordance with a general method by rearing or cultivating it to thereby form and accumulate the antibody composition and then recovering the antibody composition from the animal or plant individual.

The method for producing an antibody composition using an animal individual includes a method in which the antibody composition of interest is produced in an animal constructed by introducing a gene in accordance with a known method [American Journal of Clinical Nutrition, 63, 639S (1996); American Journal of Clinical Nutrition, 63, 627S (1996); Bio/Technology, 9, 830 (1991)].

In the case of an animal individual, an antibody composition can be produced by rearing a transgenic non-human animal into which a DNA encoding an antibody molecule is introduced to thereby form and accumulate the antibody composition in the animal, and then recovering the antibody composition from the animal. The place of the animal where the composition is produced and accumulated includes milk (Japanese Published Examined Patent Application No. 309192/88) and an egg of the animal. As the promoter used in this case, any promoter can be used, so long as it can function in an animal. Preferred examples include mammary gland cell-specific promoters such as a casein promoter, α casein promoter, β lactoglobulin promoter and whey acidic protein promoter.

The method for producing an antibody composition using a plant individual includes a method in which an antibody composition is produced by cultivating a transgenic plant into which a DNA encoding an antibody molecule is introduced by a known method [Tissue Culture, 20 (1994); Tissue Culture, 21 (1995); Trends in Biotechnology, 15, 45 (1997)] to form and accumulate the antibody composition in the plant, and then recovering the antibody composition from the plant.

Regarding purification of an antibody composition produced by a transformant into which a gene encoding an antibody molecule is introduced, for example, when the antibody composition is intraellularly expressed in a dissolved state, the cells after culturing are recovered by centrifugation, suspended in an aqueous buffer and then disrupted using a sonicator, French press, Manton Gaulin homogenizer, dynomill or the like to obtain a cell-free extract. A purified product of the antibody composition can be obtained from a supernatant obtained by centrifuging the cell-free extract, by using a general enzyme isolation purification techniques such as solvent extraction; salting out with ammonium sulfate, etc.; desalting; precipitation with an organic solvent; anion exchange chromatography using a resin such as diethylaminoethyl (DEAE)-Sepharose or DIAION HPA-75 (manufactured by Mitsubishi Chemical); cation exchange chromatography using a resin such as S-Sepharose FF (manufactured by Pharmacia); hydrophobic chromatography using a resin such as butyl-Sepharose or phenyl-Sepharose; gel filtration using a molecular sieve; affinity chromatography; chromatofocusing; electrophoresis such as isoelectric focusing; and the like which may be used alone or in combination.

Also, when the antibody composition is expressed intracellularly by forming an inclusion body, the cells are recovered, disrupted and centrifuged in the same manner, and the inclusion body of the antibody composition is recovered as a precipitation fraction. The recovered inclusion body of the antibody composition is solubilized by using a protein denaturing agent. The antibody composition is made into a normal three-dimensional structure by diluting or dialyzing the solubilized solution, and then a purified product of the antibody composition is obtained by the same isolation purification method.

When the antibody composition is secreted extracellularly, the antibody composition or derivatives thereof can be recovered from the culture supernatant. That is, the culture is treated by a technique such as centrifugation to obtain a soluble fraction, and a purified preparation of the antibody composition can be obtained from the soluble fraction by the same isolation purification method.

The thus obtained antibody composition includes an antibody, the fragment of the antibody, and a fusion protein comprising the Fc region of the antibody.

As an example for obtaining the antibody composition, a method for producing a humanized antibody composition is described below in detail, but other antibody compositions can also be obtained in a manner similar to the method.

(1) Construction of Vector for Expression of Humanized Antibody

A vector for expression of humanized antibody is an expression vector for animal cell into which genes encoding the C regions of heavy chain (H chain) and light chain (L chain) of a human antibody are inserted, which can be constructed by cloning each of genes encoding the C regions of H chain and L chain of a human antibody into an expression vector for animal cell.

The C regions of a human antibody can be the C regions of H chain and L chain of any human antibody. Examples include the C region belonging to IgG1 subclass in the H chain of a human antibody (hereinafter referred to as “hCγ1”), the C region belonging to κ class in the L chain of a human antibody (hereinafter referred to as “hCκ”), and the like.

As the genes encoding the C regions of H chain and L chain of a human antibody, a genomic DNA comprising an exon and an intron can be used and a cDNA can also be used.

As the expression vector for animal cell, any vector can be used, so long as a gene encoding the C region of a human antibody can be inserted thereinto and expressed therein. Examples include pAGE107 [Cytotechnology, 3, 133 (1990)], pAGE103 [J. Biochem., 101, 1307 (1987)], pHSG274 [Gene, 27, 223 (1984)], pKCR [Proc. Natl. Acad. Sci. USA, 78, 1527 (1981), pSGI β d24 [Cytotechnology, 4, 173 (1990)] and the like. Examples of the promoter and enhancer in the expression vector for animal cell include SV40 early promoter and enhancer [J. Biochem., 101, 1307 (1987)], Moloney mouse leukemia virus LTR promoter [Biochem. Biophys. Res. Commun., 14, 960 (1987)], immunoglobulin H chain promoter [Cell, 41, 479 (1985)] and enhancer [Cell, 33, 717 (1983)], and the like.

The vector for expression of humanized antibody can be any type; wherein genes encoding the H chain and L chain of an antibody exist on separate vectors or genes exist on the same vector (hereinafter referred to as “tandem type”). In respect of easiness of construction of a vector for expression of humanized antibody, easiness of introduction into animal cells, and balance between the expression amounts of the H and L chains of an antibody in animal cells, a tandem type of the vector for expression of humanized antibody is preferred [J. Immunol. Methods, 167, 271 (1994)]. The tandem type of the vector for expression of humanized antibody includes pKANTEX93 [Mol. Immunol, 37, 1035 (2000)], pEE18 [Hybridoma, 17, 559 (1998)] and the like.

The constructed vector for expression of humanized antibody can be used for expression of a human chimeric antibody and a human CDR-grafted antibody in animal cells.

(2) Preparation of a cDNA Encoding the V Region of an Antibody Derived from a Non-Human Animal

cDNAs encoding the V regions of H chain and L chain of an antibody derived from a non-human animal, such as a mouse antibody, can be obtained in the following manner.

A cDNA is synthesized by extracting mRNA from a hybridoma cell which produces the mouse antibody of interest. The synthesized cDNA is cloned into a vector such as a phage or a plasmid to obtain a cDNA library Each of a recombinant phage or recombinant plasmid comprising a cDNA encoding the V region of H chain and a recombinant phage or recombinant plasmid comprising a cDNA encoding the V region of L chain is isolated from the library by using a C region part or a V region part of an existing mouse antibody as the probe. The full nucleotide sequences of the V regions of H chain and L chain of the mouse antibody of interest on the recombinant phage or recombinant plasmid are determined, and the fall amino acid sequences of the V regions of H chain and L chain are deduced from the nucleotide sequences.

As the non-human animal, any animal such as mouse, rat, hamster, rabbit or the like can be used so long as a hybridoma cell can be produced therefrom.

The method for preparing total RNA from a hybridoma cell includes the guanidine thiocyanate-cesium trifluoroacetate method [Methods in Enzymology, 154, 3 (1987)] and the like. The method for preparing mRNA from total RNA includes an oligo(dT)-immobilized cellulose column method (Molecular Cloning: A Laboratory Manual, Cold Spring Harbor Lab. Press New York, 1989) and the like. In addition, a kit for preparing mRNA from a hybridoma cell includes Fast Track mRNA Isolation Kit (manufactured by Invitrogen), Quick Prep mRNA Purification Kit (manufactured by Pharmacia) and the like.

The method for synthesizing cDNA and preparing a cDNA library includes the usual methods (Molecular Cloning: A Laboratory Manual, Cold Spring Harbor Lab. Press New York 1989, Current Protocols in Molecular Biology, Supplement 1-34); methods using a commercially available kit such as SuperScript™ Plasmid System for cDNA Synthesis and Plasmid Cloning (manufactured by GIBCO BRL) and ZAP-cDNA Synthesis Kit (manufactured by Stratagene); and the like.

In preparing the cDNA library, the vector into which a cDNA synthesized using mRNA extracted from a hybridoma cell as the template is inserted can be any vector so long as the cDNA can be inserted. Examples include ZAP Express [Strategies, 5, 58 (1992)], pBluescript II SK(+) [Nucleic Acids Research, 17, 9494 (1989)], λzapII (manufactured by Stratagene), λgt10 and λgt11 [DNA Cloning, A Practical Approach, I, 49 (1985)], Lambda BlueMid (manufactured by Clontech), λExCell and pT7T3 18U (manufactured by Pharmacia), pcD2 [Mol. Cell. Biol., 3, 280 (1983)], pUC18 [Gene, 33, 103 (1985)] and the like.

As Escherichia coli into which the cDNA library constructed from a phage or plasmid vector is introduced, any Escherichia coli can be used, so long as the cDNA library can be introduced, expressed and maintained. Examples include XL1-Blue MRF′ [Strategies, 5, 81 (1992)], C600 [Genetics, 39, 440 (1954)], Y1088 and Y1090 [Science, 222, 778 (1983)], NM522 [J. Mol. Biol., 166, 1 (1983)], K802 [J. Mol. Biol., 16, 118 (1966)], 3M105 [Gene, 38, 275 (1985)] and the like.

As the method for selecting a cDNA clone encoding the V regions of H chain and L chain of an antibody derived from a non-human animal from the cDNA library, a colony hybridization or a plaque hybridization using an isotope- or fluorescence-labeled probe can be used (Molecular Cloning, Second Edition, Cold Spring Harbor Lab. Press New York, 1989). The cDNA encoding the V regions of H chain and L chain can also be prepared by preparing primers and carrying out polymerase chain reaction (hereinafter referred to as “PCR”; Molecular Cloning, Second Edition, Cold Spring Harbor Lab. Press New York, 1989; Current Protocols in Molecular Biology, Supplement 1-34) using a cDNA synthesized from mRNA or a cDNA library as the template.

The nucleotide sequences of the cDNAs can be determined by digesting the selected cDNAs with appropriate restriction enzymes, cloning the fragments into a plasmid such as pBluescript SK(−) (manufactured by Stratagene), carrying out the reaction of a generally used nucleotide sequence analyzing method such as the dideoxy method [Proc. Natl. Acad. Sci. USA, 74, 5463 (1977)] of Sanger et al., and then analyzing the clones using an automatic nucleotide sequence analyzer such as A.L.F. DNA Sequencer (manufactured by Pharmacia).

Whether or not the obtained cDNAs are encoding the full length of amino acid sequences of the V regions of H chain and L chain of the antibody containing a secretory signal sequence can be confirmed by deducing the fill amino acid sequences of the V regions of H chain and L chain from the determined nucleotide sequence and comparing them with the full length amino acid sequences of the V regions of H chain and L chain of known antibodies [Sequences of Proteins of Immunological Interest, US Dep. Health and Human Services (1991)].

Furthermore, when the amino acid sequence of an antibody variable region or the nucleotide sequence of a DNA encoding the variable region is known, the cDNA can be prepared according to the following method.

When the amino acid sequence is known, the amino acid sequence is converted to a DNA sequence based on frequency of codon usage [Sequences of Proteins of Immunological Interest, US Dep. Health and Human Services (1991)], several synthetic DNAs having a length of about 100 bases are synthesized based on the designed DNA sequence, and PCR is carried out by using the DNAs to prepare the cDNA. When the nucleotide sequence is known, several synthetic DNAs having a length of about 100 bases are synthesized based on the designed DNA sequence, and PCR is carried out by using the DNAs to prepare the cDNA.

(3) Analysis of the Amino Acid Sequence of the V Region of an Antibody Derived from a Non-Human Animal

Regarding the full length of the amino acid sequences of the V regions of H chain and L chain of an antibody comprising a secretory signal sequence, the length of the secretory signal sequence and the N-terminal amino acid sequences can be deduced and subgroups to which they belong can also be found, by comparing them with the full length of the amino acid sequences of the V regions of H chain and L chain of known antibodies [Sequences of Proteins of Immunological Interest US Dep. Health and Human Services, (1991)]. In addition, the amino acid sequences of each CDR of the V regions of H chain and L chain can also be found by comparing them with the amino acid sequences of the V regions of H chain and L chain of known antibodies [Sequences of Proteins of Immunological Interest, US Dep. Health and Human Services, (1991)].

(4) Construction of Vector for Expression of Human Chimeric Antibody

A vector for expression of human chimeric antibody can be constructed by cloning cDNAs encoding the V regions of H chain and L chain of an antibody derived from a non-human animal into upstream of genes encoding the C regions of H chain and L chain of a human antibody in the vector for humanized antibody expression described in item 3(1). For example, a vector for expression of human chimeric antibody can be constructed by linking each of cDNAs encoding the V regions of H chain and L chain of an antibody derived from a non-human animal to a synthetic DNA comprising nucleotide sequences at the 3′-terminals of the V regions of H chain and L chain of an antibody derived from a non-human animal and nucleotide sequences at the 5′-terminals of the C regions of H chain and L chain of a human antibody and also having a recognition sequence of an appropriate restriction enzyme at both terminals, and by cloning them into upstream of genes encoding the C regions of H chain and L chain of a human antibody contained in the vector for humanized antibody expression constructed as described in item 3(1) in the form suitable for expression.

(5) Construction of cDNA Encoding the V Region of a Human CDR-Grafted Antibody

cDNAs encoding the V regions of H chain and L chain of a human CDR-grafted antibody can be obtained as follows. First, amino acid sequences of the frameworks (hereinafter referred to as “FR”) of the V regions of H chain and L chain of a human antibody for grafting CDR of the V regions of H chain and L chain of an antibody derived from a non-human animal is selected. As the amino acid sequences of FRs of the V regions of H chain and L chain of a human antibody, any amino acid sequences can be used so long as they are derived from a human antibody. Examples include amino acid sequences of FRs of the V regions of H chain and L chain of human antibodies registered at databases such as Protein Data Bank; amino acid sequences common in each subgroup of FRs of the V regions of H chain and L chain of human antibodies [Sequences of Proteins of Immunological Interest, US Dep. Health and Human Services (1991)]; and the like. In order to produce a human CDR-grafted antibody having potent activity, it is preferable to select an amino acid sequence having a homology as high as possible (at least 60% or more) with amino acid sequences of the V regions of H chain and L chain of an antibody of interest derived from a non-human animal.

Next, the amino acid sequences of CDRs of the V regions of H chain and L chain of the antibody of interest derived from a non-human animal are grafted to the selected amino acid sequences of FRs of the V regions of H chain and L chain of a human antibody to design amino acid sequences of the V regions of H chain and L chain of the human CDR-grafted antibody. The designed amino acid sequences are converted into DNA sequences by considering the frequency of codon usage found in nucleotide sequences of antibody genes [Sequences of Proteins of Immunological Interest, US Dep. Health and Human Services (1991)] and the DNA sequences encoding the amino acid sequences of the V regions of H chain and L chain of the human CDR-grafted antibody are designed. Based on the designed DNA sequences, several synthetic DNAs having a length of about 100 bases are synthesized, and PCR is carried out by using them. In this case, it is preferable in each of the H chain and the L chain that 4 to 6 synthetic DNAs are designed in view of the reaction efficiency of PCR and the lengths of DNAs which can be synthesized.

Also, they can be easily cloned into the vector for humanized antibody expression constructed in item 3(1) by introducing recognition sequences of an appropriate restriction enzyme into the 5′-terminals of the synthetic DNA present on both terminals. After the PCR, the amplified product is cloned into a plasmid such as pBluescript SK(−) (manufactured by Stratagene) or the like, and the nucleotide sequences are determined by the method in item 3(2) to thereby obtain a plasmid having DNA sequences encoding the amino acid sequences of the V regions of H chain and L chain of the desired human CDR-grafted antibody.

(6) Construction of Vector for Human CDR-Grafted Antibody Expression

A vector for human CDR-grafted antibody expression can be constructed by cloning the cDNAs encoding the V regions of H chain and L chain of the human CDR-grafted antibody constructed in item 3(5) into upstream of the gene encoding C regions of H chain and L chain of a human antibody in the vector for humanized antibody expression described in item 3(1). For example, the vector for human CDR-grafted antibody expression can be constructed by introducing recognizing sequences of an appropriate restriction enzyme into the 5′-terminals of both terminals of a synthetic DNA fragment, among the synthetic DNA fragments which are used when PCR is carried out in item 3(5) for constructing the V regions of H chain and L chain of the human CDR-grafted antibody, so that they are cloned into upstream of the genes encoding the C regions of H chain and L chain of a human antibody in the vector for humanized antibody expression described in item 3(1) in such a manner that they can be expressed in a suitable form.

(7) Stable Production of a Humanized Antibody

A transformant capable of stably producing a human chimeric antibody and a human CDR-grafted antibody (both hereinafter referred to as “humanized antibody”) can be obtained by introducing the vector for expression of humanized antibody described in items 3(4) or (6) into an appropriate animal cell.

The method for introducing a vector for expression of humanized antibody into an animal cell includes electroporation [Japanese Published Examined Patent Application No. 257891/90, Cytotechnology, 3, 133 (1990)] and the like.

As the animal cell into which a vector for expression of humanized antibody is introduced, any cell can be used so long as it is an animal cell which can produce the humanized antibody.

Examples include mouse myeloma cells such as NS0 cell and SP2/0 cell; Chinese hamster ovary cells such as CHO/dhfr⁻ cell and CHO/DG44 cell; rat myeloma such as YB2/0 cell and IR983F cell; BHK cell derived from a Syrian hamster kidney; a human myeloma cell such as Namalwa cell; and the like. A Chinese hamster ovary cell CHO/DG44 cell and a rat myeloma YB2/0 cell are preferred.

After introduction of the vector for expression of humanized antibody, a transformant capable of stably producing the humanized antibody can be selected, in accordance with the method disclosed in Japanese Published Examined Patent Application No. 257891/90 using a medium for animal cell culture comprising an agent such as G418 sulfate (hereinafter referred to as “G418”; manufactured by SIGMA). As the medium for animal cell culture, RPMI 1640 medium (manufactured by Nissui Pharmaceutical), GIT medium (manufactured by Nihon Pharmaceutical), EX-CELL 302 medium (manufactured by JRH), IMDM medium (manufactured by GIBCO BRL), Hybridoma-SFM medium (manufactured by GIBCO BRL), media obtained by adding various additives such as fetal bovine serum (hereinafter referred to as “FCS”) to these media, and the like can be mentioned. The humanized antibody can be produced and accumulated in the culture medium by culturing the obtained transformant in a medium. The production and antigen binding activity of the humanized antibody in the culture medium can be measured by a method such as enzyme-linked immunosorbent assay [hereinafter referred to as “ELISA”; Antibodies: A Laboratory Manual, Cold Spring Harbor Laboratory, Chapter 14 (1998), Monoclonal Antibodies: Principles and Practice, Academic Press Limited (1996)] or the like. Also, the production of the humanized antibody by the transformant can be increased by using a DHFR gene amplification system in accordance with the method disclosed in Japanese Published Examined Patent Application No. 257891/90.

The humanized antibody can be purified from a culture supernatant of the transformant by using a protein A column [Antibodies. A laboratory Manual, Cold Spring Harbor Laboratory, Chapter 8 (1988), Monoclonal Antibodies: Principles and Practice, Academic Press Limited (1996)]. In addition, purification methods generally used for the purification of proteins can also be used. For example, the purification can be carried out through the combination of gel filtration, ion exchange chromatography and ultrafiltration. The molecular weight of the H chain, L chain and antibody molecule as a whole of the purified humanized antibody, respectively, can be measured, e.g., by polyacrylamide gel electrophoresis [hereinafter referred to as “SDS-PAGE”; Nature, 227, 680 (1970)], Western blotting [Antibodies: A Laboratory Manual, Cold Spring Harbor Laboratory, Chapter 12 (1988), Monoclonal Antibodies: Principles and Practice, Academic Press Limited (1996)] or the like.

Thus, methods for producing an antibody composition using an animal cell as the host have been described, but, as described above, the antibody composition can also be produced by a yeast, an insect cell, a plant cell, an animal individual or a plant individual by the same methods as the animal cell.

When a host cell has the ability to express an antibody molecule, the antibody composition of the present invention can be produced by preparing a cell expressing an antibody molecule by using the method described in item 1, culturing the cell and then purifying the antibody composition of interest from the resulting culture.

4. Activity Evaluation of the Antibody Composition

As the method for measuring the amount of the protein of the purified antibody composition, the activity to bind to an antigen and the effector function of the purified antibody composition, the known methods described in Monoclonal Antibodies, Antibody Engineering and the like can be used.

For example, when the antibody composition is a humanized antibody, the binding activity with an antigen and the binding activity with an antigen-positive cultured cell line can be measured by methods such as ELISA and an immunofluorescent method [Cancer Immunol. Immunother., 36, 373 (1993)]. The cytotoxic activity against an antigen-positive cultured cell line can be evaluated by measuring CDC activity, ADCC activity [Cancer Immunol. Immunother., 36, 373 (1993)] and the like.

Also, safety and therapeutic effect of the antibody composition in human can be evaluated by using an appropriate model of animal species relatively close to human, such as Macaca fascicularis.

5. Analysis of Sugar Chains in the Antibody Composition

The sugar chain structure of the antibody molecule expressed in various cells can be analyzed in accordance with the general analysis of the sugar chain structure of a glycoprotein. For example, the sugar chain which is bound to IgG molecule comprises a neutral sugar such as galactose, mannose or fucose, an amino sugar such as N-acetylglucosamine, and an acidic sugar such as sialic acid, and can be analyzed by a method such as a sugar chain structure analysis using sugar composition analysis, two dimensional sugar chain mapping or the like.

(1) Analysis of Neutral Sugar and Amino Sugar Compositions

The sugar chain of the antibody composition can be analyzed by carrying out acid hydrolysis of sugar chains with an acid such as trifluoroacetic acid to release a neutral sugar or an amino sugar and measuring the composition ratio.

Examples include a method using a sugar composition analyzer (BioLC) manufactured by Dionex. The BioLC is an apparatus which analyzes a sugar composition by EPAEC-PAD (high performance anion-exchange chromatography-pulsed amperometric detection) [J. Liq. Chromatogr., 6, 1577 (1983)].

The composition ratio can also be analyzed by a fluorescence labeling method using 2-aminopyridine. Specifically, the compositional ratio can be calculated in accordance with a known method [Agric. Biol. Chem., 55(1) 283-284 (1991)], by labeling an acid-hydrolyzed sample with a fluorescence with 2-aminopyridylation and then analyzing the composition by HPLC.

(2) Analysis of Sugar Chain Structure

The sugar chain structure of the antibody molecule can be analyzed by the two dimensional sugar chain mapping method [Anal. Biochem, 171, 73 (1988), Biochemical Experimentation Methods 23—Methods for Studying Glycoprotein Sugar Chains (Japan Scientific Societies Press) edited by Reiko Takahashi (1989)]. The two dimensional sugar chain mapping method is a method for deducing a sugar chain structure by, e.g., plotting the retention time or elution position of a sugar chain by reverse phase chromatography as the X axis and the retention time or elution position of the sugar chain by normal phase chromatography as the Y axis, respectively, and comparing them with such results of known sugar chains.

Specifically, sugar chains are released from an antibody by subjecting the antibody to hydrazinolysis, and the released sugar chain is subjected to fluorescence labeling with 2-aminopyridine (hereinafter referred to as “PA”) [J. Biochem., 95, 197 (1984)], and then the sugar chains are separated from an excess PA-treating reagent by gel filtration, and subjected to reverse phase chromatography. Thereafter, each peak of the separated sugar chains are subjected to normal phase chromatography. The sugar chain structure can be deduced by plotting the results on a two dimensional sugar chain map and comparing them with the spots of a sugar chain standard (manufactured by Takara Shuzo) or a literature [Anal. Biochem., 171, 73 (1988)].

The structure deduced by the two dimensional sugar chain mapping method can be confirmed by further carrying out mass spectrometry such as MALDI-TOF-MS of each sugar chain.

6. Immunological Determination Method for Discriminating the Sugar Chain Structure of an Antibody Molecule

An antibody composition comprises various antibody molecules in which sugar chains binding to the Fc region of the antibody are different in structure. In the antibody composition of the present invention, the ratio of a sugar chain in which fucose is not bound to N-acetylglucosamine in the reducing end in the sugar chain is 20% or more among the total complex N-glycoside-linked sugar chains binding to the Fc region in the antibody composition, and the antibody composition has potent ADCC activity. The antibody composition can be identified by using the method for analyzing the sugar chain structure of an antibody molecule described in item 5. Also, it can be identified by an immunological determination method using a lectin.

The sugar chain structure of an antibody molecule can be identified by the immunological determination method using a lectin in accordance with the known immunological determination method such as Western staining, IRA (radioimmunoassay), VIA (viroimmunoassay), EIA (enzymoimmunoassay), FIA (fluoroimmunoassay) and MIA (metalloimmunoassay) described in Monoclonal Antibodies: Principles and Applications, Wiley-Liss, Inc. (1995); Immunoassay, 3rd Ed., Igakushoin (1987); Enzyme Antibody Method, Revised Edition, Gakusai Kikaku (1985); and the like.

A lectin which recognizes the sugar chain structure of an antibody molecule comprised in an antibody composition is labeled, and the labeled lectin is allowed to react with an antibody composition as a sample. Then, the amount of the complex of the labeled lectin with the antibody molecule is measured.

The lectin for identifying the sugar chain structure of an antibody molecule includes WGA (wheat-germn agglutinin derived from T vilgaris), ConA (cocanavalin A derived from C. ensiformis), RIC (a toxin derived from R. communis), L-PHA (leucoagglutinin derived from P. vulgaris), LCA (lentil agglutinin derived from L. culinais), PSA (pea lectin derived from P. sativum), AAL (Aleuria aurantia lectin), ACL (Amaranihus cauds lectin), BPL (Bauhinia purpurea lectin), DSL (Datura stramonium lectin), DBA (Dolichos biflorus agglutinin), EBL (elderberry balk lectin), ECL (Erythrina cristagalli lectin), EEL (Euonymus eoropaeus lectin), GNL (Galanthus nivalis lectin), GSL (Griffonia simplicifolia lectin), HPA (Helix pomatia agglutinin), HHL (Hippeastrum hybrid lectin), Jacalin, LTL (Lotus tetragonolobus lectin), LEL (Lycopersicon esculentum lectin), MAL (Maackia amurensis lectin), MPL (Machura pomifera lectin), NPL (Narcissus pseudonarcissus lectin), PNA (peanut agglutinin), EPHA (Phaseolus vulgaris erythroagglutinin), PTL (Psophocarpus teiragonolobus lectin), RCA (Ricinus communis agglutinin), STL (Solamum tuberosum lectin), SJA (Sophora japonica agglutinin), SBA (soybean agglutinin), UEA (Ulex europaeus agglutinin), VVL (Vicia villosa lectin) and WFA (Wisteria floribunda agglutinin).

It is preferable to use a lectin which specifically recognizes a sugar chain structure wherein fucose binds to the N-acetylglucosamine in the reducing end in the complex N-glycoside-linked sugar chain. Examples include Lens culinaris lectin LCA (lentil agglutinin derived from Lens culinaris), pea lectin PSA (pea lectin derived from Pisum sativum), broad bean lectin VFA (agglutinin derived from Vicia faba) and Aleuria aurantia lectin AAL (lectin derived from Aleuria aurantia).

7. Application of the Antibody Molecule of the Present Invention

Since the antibody composition of the present invention specifically binds to CD20 and has potent antibody-dependent cell-mediated cytotoxic activity, it is useful for preventing and treating various diseases relating to CD20-expressing cells such as cancers.

In the case of cancers, namely malignant tumors, cancer cells grow, and, for example, particular B cells abnormally grow in B cell lymphoma. General anti-tumor agents inhibit the growth of cancer cells. In contrast, an antibody having potent antibody-dependent cell-mediated cytotoxic activity can cure cancers by injuring cancer cells through its cell killing effect, and therefore, it is more effective as a therapeutic agent which express the antingen than the general anti-tumor agents. Particularly, in the therapeutic agent for cancers, an anti-tumor effect of an antibody medicament alone is insufficient at the present so that combination therapy with chemotherapy has been carried out [Science, 280, 1197 (1998)). If more potent anti-tumor effect is found by the antibody composition of the present invention alone, the dependency on chemotherapy will be decreased and side effects will be reduced.

The antibody composition of the present invention can be administered as a therapeutic agent alone. Generally, it is preferable to mix the antibody composition with at least one pharmaceutical acceptable carrier and provide it as a pharmaceutical formulation produced by an appropriate method well known in the technical field of manufacturing pharmacy.

It is preferable to select a route of administration which is the most effective in treatment. Examples include oral administration and parenteral administration such as buccal, tracheal, rectal, subcutaneous, intramuscular and intravenous. In an antibody preparation, intravenous administration is preferable.

The dosage form includes sprays, capsules, tablets, granules, syrups, emulsions, suppositories, injections, ointments, tapes and the like.

Examples of the pharmaceutical preparation suitable for oral administration include emulsions, syrups, capsules, tablets, powders, granules and the like.

Liquid preparations, such as emulsions and syrups, can be produced using, as additives, water, saccharides such as sucrose, sorbitol and fucose; glycols such as polyethylene glycol and propylene glycol; oils such as sesame oil olive oil and soybean oil; antiseptics such as p-hydroxybenzoic acid esters; flavors such as strawberry flavor and peppermint; and the like.

Capsules, tablets, powders, granules and the like can be produced using, as additive, excipients such as lactose, glucose, sucrose and mannitol; disintegrating agents such as starch and sodium arginate; lubricants such as magnesium stearate and talc; binders such as polyvinyl alcohol, hydroxypropylcellulose and gelatin; surfactants such as fatty acid ester; plasticizers such as glycerine; and the like.

The pharmaceutical preparation suitable for parenteral administration includes injections, suppositories, sprays and the like.

Injections may be prepared using a carrier such as a salt solution, a glucose solution or a mixture of both thereof or the like. Also, powdered injections can be prepared by freeze-drying the antibody composition in the usual way and adding sodium chloride thereto.

Suppositories may be prepared using a carrier such as cacao butter, hydrogenated fat or carboxylic acid.

Also, sprays may be prepared using the antibody composition as such or using a carrier which does not stimulate the buccal or airway mucous membrane of the patient and can facilitate absorption of the antibody composition by dispersing it as fine particles.

The carrier includes lactose, glycerine and the like. Depending on the properties of the antibody composition and the carrier, it is possible to produce pharmaceutical preparations such as aerosols and dry powders. In addition, the components exemplified as additives for oral preparations can also be added to the parenteral preparations.

Although the clinical dose or the frequency of administration varies depending on the objective therapeutic effect, administration method, treating period, age, body weight and the like, it is usually 10 μg/kg to 20 mg/kg per day and per adult.

Also, as the method for examining antitumor effect of the antibody composition against various tumor cells, in vitro tests include CDC activity measuring method, ADCC activity measuring method, and the like; and in vivo tests include antitumor experiments using a tumor system in an experimental animal such as a mouse, and the like.

CDC activity and ADCC activity measurements and antitumor experiments can be carried out in accordance with the methods described in Cancer Immunology Immunotherapy, 36, 373 (1993); Cancer Research, 54, 1511 (1994) and the like.

The present invention will be described below in detail based on Examples; however, Examples are only simple illustrations of the present invention, and the scope of the present invention is not limited thereto.

### EXAMPLE 1

Preparation of an Anti-CD20 Human Chimeric Antibody:

1. Preparation of Anti-CD20 Vector for Expression of Human Chimeric Antibody

(1) Construction of a cDNA Encoding the V Region of L Chain of an Anti-CD20 Mouse Monoclonal Antibody

A cDNA (represented by SEQ ID NO:11) encoding the amino acid sequence of the V region of L chain (hereinafter referred to as “VL”) of an anti-CD20 mouse monoclonal antibody 2B8 described in WO 94/11026 was constructed using PCR as follows.

First, binding nucleotide sequences of primers for amplification at the time of the PCR and restriction enzyme recognizing sequences for cloning into a vector for humanized antibody expression were added to the 5′-terminal and 3′-terminal of the nucleotide sequence of the VL described in WO 94/11026. A designed nucleotide sequence was divided from the 5′-terminal side into a total of 6 nucleotide sequences each having about 100 bases (adjacent nucleotide sequences are designed such that their termini have an overlapping sequence of about 20 bases), and 6 synthetic DNA fragments, actually those represented by SEQ ID NOs:15, 16, 17, 18, 19 and 20, were prepared from them in alternate order of a sense chain and an antisense chain (consigned to GENSET).

Each oligonucleotide was added to 50 μl of a reaction mixture [KOD DNA polymerase-attached PCR Buffer #1 (manufactured by TOYOBO), 0.2 mM dNTPs, 1 mM magnesium chloride, 0.5 μM M13 primer M4 (manufactured by Takara Shuzo) and 0.5 μM M13 primer RV (manufactured by Takara Shuzo)] to give a final concentration of 0.1 μM, and using a DNA thermal cycler GeneAmp PCR System 9600 (manufactured by Perkin Elmer), the reaction was carried out by heating at 94° C. for 3 minutes, adding 2.5 units of KOD DNA Polymerase (manufactured by TOYOBO) thereto, subsequent 25 cycles of heating at 94° C. for 30 seconds, 55° C. for 30 seconds and 74° C. for 1 minute as one cycle and then further heating at 72° C. for 10 minutes. After 25 μl of the reaction mixture was subjected to agarose gel electrophoresis, a VL PCR product of about 0.44 kb was recovered by using QIAquick Gel Extraction Kit (manufactured by QIAGEN).

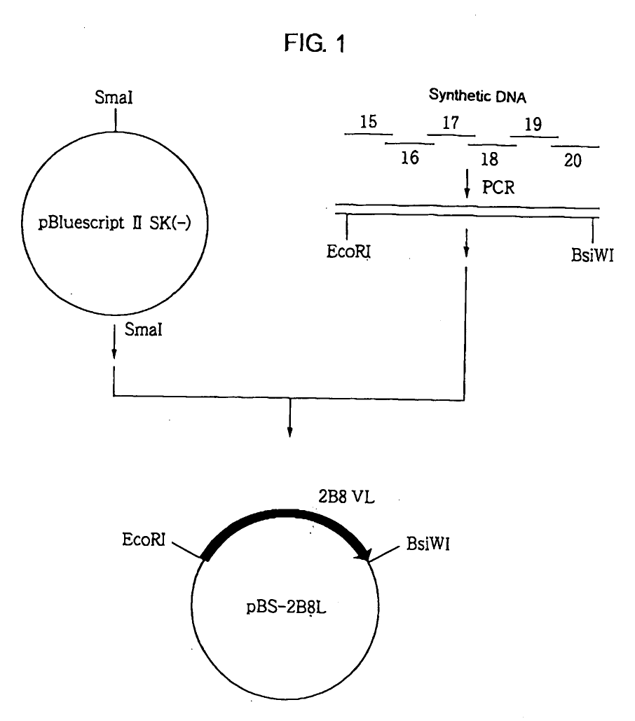

Next, 0.1 μg of a DNA fragment obtained by digesting a plasmid pbluescript II SK(−) (manufactured by Stratagene) with a restriction enzyme SmaI (manufactured by Takara Shuzo) and about 0.1 μg of the PCR product obtained in the above were added to sterile water to adjust the total volume to 7.5 μl and then 7.5 μl of solution I of TAKARA ligation kit ver. 2 (manufactured by Takara Shuzo) and 0.3 μl of the restriction enzyme SmaI (manufactured by Takara Shuzo) were added thereto to carry out the reaction at 22° C. for 2 hours. Using the recombinant plasmid DNA solution obtained in this manner, E. coli DH5α (manufactured by TOYOBO) was transformed. Each plasmid DNA was prepared from the transformant clones and allowed to react using BigDye Terminator Cycle Sequencing Ready Reaction Kit v2.0 (manufactured by Applied Biosystems) in accordance with the instructions attached thereto, and then the nucleotide sequence was analyzed by a DNA sequencer ABI PRISM 377 manufactured by the same company. In this manner, the plasmid pBS2B8L shown in FIG. 1 having the objective nucleotide sequence was obtained.

(2) Construction of a EDNA Encoding the V Region of H Chain of an Anti-CD20 Mouse Monoclonal Antibody

A cDNA (represented by SEQ ID NO:13) encoding the amino acid sequence of the V region of H chain (hereinafter referred to as “VH”) of the anti-CD20 mouse monoclonal antibody 2B8 described in WO 94/11026 was constructed using PCR as follows.

First, binding nucleotide sequences of primers for amplification at the time of the PCR and a restriction enzyme recognizing sequence for cloning into a vector for humanized antibody expression were added to the 5′-terminal and 3′-terminal of the nucleotide sequence of the VH described in WO 94/11026. A designed nucleotide sequence was divided from the 5′-terminal side into a total of 6 nucleotide sequences each having about 100 bases (adjacent nucleotide sequences are designed such that their termini have an overlapping sequence of about 20 bases), and 6 synthetic DNA fragments, actually those represented by SEQ ID NOs:25, 26, 27, 28, 29 and 30, were prepared from them in alternate order of a sense chain and an antisense chain (consigned to GENSET).

Each oligonucleotide was added to 50 μl of a reaction mixture [KOD DNA polymerase-PCR Buffer #1 (manufactured by TOYOBO), 0.2 mM dNTPs, 1 mM magnesium chloride, 0.5 μM M13 primer M4 (manufactured by Takara Shuzo) and 0.5 μM M13 primer RV (manufactured by Takara Shuzo)] to give a final concentration of 0.1 μM, and using a DNA thermal cycler GeneAmp PCR System 9600 (manufactured by Perkin Elmer), the reaction was carried out by heating at 94° C. for 3 minutes, adding 2.5 units of KOD DNA Polymerase (manufactured by TOYOBO), subsequent 25 cycles of heating at 94° C. for 30 seconds, 55° C. for 30 seconds and 74° C. for 1 minute as one cycle and then heating at 72° C. for 10 minutes. After 25 μl of the reaction mixture was subjected to agarose gel electrophoresis, a VH PCR product of about 0.49 kb was recovered by using QIAquick Gel Extraction Kit (manufactured by QIAGEN).

Next, 0.1 μg of a DNA fragment obtained by digesting the plasmid pBluescript II SK(−) (manufactured by Stratagene) with the restriction enzyme SmaI (manufactured by Takara Shuzo) and about 0.1 μg of the PCR product obtained in the above were added to sterile water to adjust the total volume to 7.5 μJ, and then 7.5 μl of solution I of TAKARA ligation kit ver. 2 (manufactured by Takara Shuzo) and 0.3 μl of the restriction enzyme SmaI (manufactured by Takara Shuzo) were added thereto to carry out the reaction at 22° C. overnight.

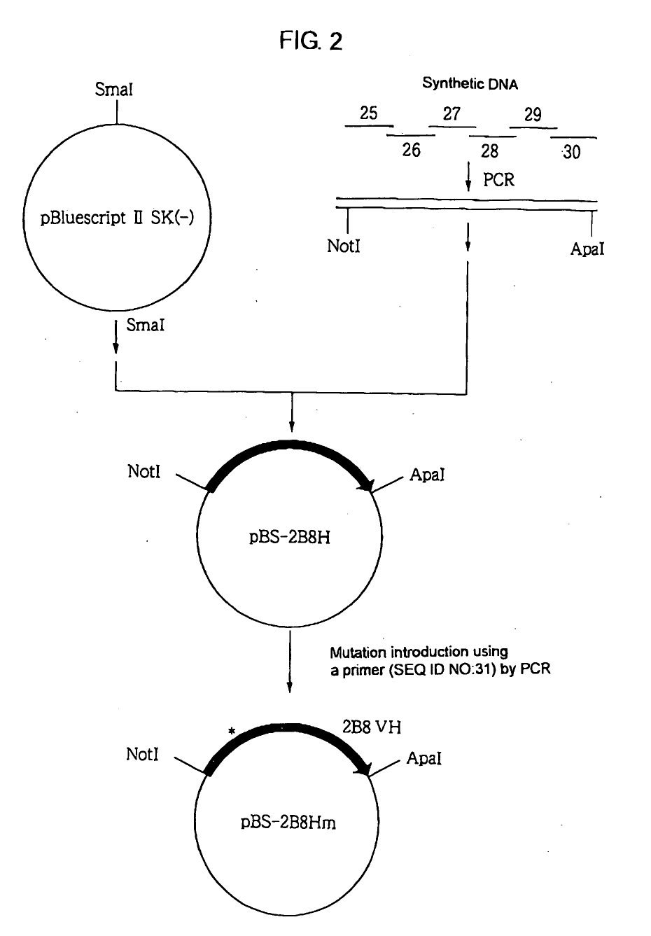

Using the recombinant plasmid DNA solution obtained in this manner, E. coli DH5α (manufactured by TOYOBO) was transformed. Each plasmid DNA was prepared from the transformant clones and allowed to react using BigDye Terminator Cycle Sequencing Ready Reaction Kit v2.0 (manufactured by Applied Biosystems) in accordance with the manufacture's instructions attached thereto, and then the nucleotide sequence was analyzed by the DNA sequencer ABI PRISM 377 manufactured by the same company. In this manner, the plasmid pBS-2B8H shown in FIG. 2 comprising the objective nucleotide sequence was obtained.

Next, in order to substitute the amino acid residue at position 14 from Ala to Pro, the synthetic DNA shown in SEQ ID NO:31 was designed, and base substitution was carried out by PCR using LA PCR in vitro Mutagenesis Primer Set for pBluescript II (manufactured by Takara Shuzo) as follows. After 50 μl of a reaction mixture [LA PCR Buffer IE (manufactured by Takara Shuzo), 2.5 units of TaKaRa LA Taq, 0.4 mM dNTPs, 2.5 mM magnesium chloride, 50 nM T3 BcaBEST Sequencing primer (manufactured by Takara Shuzo) and 50 nM of the primer for mutagenesis (SEQ ID NO:31, manufactured by GENSET)] containing 1 ng of the plasmid pBS-2B8H was prepared, the PCR was carried out by using a DNA thermal cycler GeneAmp PCR System 9600 (manufactured by Perkdn Elmer) by 25 cycles of heating at 94° C. for 30 seconds, 55° C. for 2 minutes and 72° C. for 1.5 minutes as one cycle. After 30 μl of the reaction mixture was subjected to agarose gel electrophoresis, a PCR product of about 0.44 kb was recovered by using QIAquick Gel Extraction Kit (manufactured by QIAGEN) and made into 30 μl of an aqueous mixture. In the same manner, PCR was carried out by using 50 μl of a reaction mixture [LA PCR Buffer II (manufactured by Takara Shuzo), 2.5 units of TaKaRa LA Taq, 0.4 mM dNTPs, 2.5 mM magnesium chloride, 50 nM 17 BcaBEST Sequencing primer (manufactured by Takara Shuzo) and 50 nM MUT B1 primer (manufactured by Takara Shuzo)] containing 1 ng of the plasmid pBS-2B8R After 30 μl of the reaction mixture was subjected to agarose gel electrophoresis, a PCR product of about 0.63 kb was recovered by using QIAquick Gel Extraction Kit (manufactured by QIAGEN) and made into 30 μl of aqueous solution. Next, 0.5 μl of each of 0.44 kb PCR product and 0.63 kb PCR product thus obtained were added to 47.5 μl of a reaction mixture [LA PCR Buffer II (manufactured by Takara Shuzo), 0.4 mM dNTPs, and 2.5 mM magnesium chloride], and using a DNA thermal cycler GeneAmp PCR System 9600 (manufactured by Perkin Elmer), annealing of the DNA was carried out by heating the reaction mixture at 90° C. for 10 minutes, cooling it to 37° C. over 60 minutes and then keeping it at 37° C. for 15 minutes. After carrying out the reaction at 72° C. for 3 minutes by adding 2.5 units of TaKaRa LA Taq (manufactured by Takara Shuzo), 10 pmol of each of T3 BcaBEST Sequencing primer (manufactured by Takara Shuzo) and T BcaBEST Sequencing primer (manufactured by Takara Shuzo) were added thereto to make the volume of the reaction mixture to 50 μl, which was subjected to 10 cycles of heating 94° C. for 30 seconds, 55° C. for 2 minutes and 72° C. for 1.5 minutes as one cycle. After 25 μl of the reaction mixture was purified using QIA quick PCR purification kit (manufactured by QIAGEN), a half volume thereof was allowed to react at 37° C. for 1 hour using 10 units of a restriction enzyme KpnI (manufactured by Takara Shuzo) and 10 units of a restriction enzyme SacI (manufactured by Takara Shuzo). The reaction mixture was fractionated by using agarose gel electrophoresis to recover a KnI-SacI fragment of about 0.59 kb.

Net, 1 μg of pBluescript II SK(−) (manufactured by Stratagene) was allowed to react at 37° C. for 1 hour using 10 units of the restriction enzyme KpnI (manufactured by Takara Shuzo) and 10 units of the restriction enzyme SacI (manufactured by Takara Shuzo), and then the reaction mixture was subjected to agarose gel electrophoresis to recover a KpnI-SacI fragment of about 2.9 kb.

The PCR product-derived KpnI-SacI fragment and plasmid pBluescript II SK(−)-derived KpnI-SacI fragment thus obtained were ligated by using Solution I of DNA Ligation Kit Ver 2 (manufactured by Takara Shuzo) in accordance with the manufacture's instructions attached thereto, Using the recombinant plasmid DNA solution obtained in this manner, E. coli DH5α (manufactured by TOYOBO) was transformed. Each plasmid DNA was prepared from the transformant clones, and allowed to react by using BigDye Terminator Cycle Sequencing Ready Reaction Kit v2.0 (manufactured by Applied Biosystems) in accordance with the manufacturers instructions attached thereto, and then the nucleotide sequence was analyzed by the DNA sequencer ABI PRISM 377 manufactured by the same company.

In this manner, the plasmid pBS-2B8Hm shown in FIG. 2 comprising the objective nucleotide sequence was obtained,

(3) Construction of an Anti-CD20 Vector for Expression of Human Chimeric Antibody

By using pKTEX93, a vector for expression of humanized antibody, (Mol. Immunol, 37, 1035, 2000) and the plasmids pBS-2B8L and pBS-2B8Hm obtained in items 1(1) and (2) of Example 1, an anti-CD20 human chimeric antibody (hereinafter referred to as “anti-CD20 chimeric antibody”) expression vector pKANTEX2B8P was constructed as follows.

After 2 μg of the plasmid pBS-2B8L obtained in item 1(1) in Example 1 was allowed to react at 55° C. for 1 hour using 10 units of a restriction enzyme BsiWI (manufactured by New England Biolabs), followed by reaction at 37° C. for 1 hour using 10 units of a restriction enzyme EcoRI (manufactured by Takara Shuzo). The reaction mixture was fractionated by agarose gel electrophoresis to recover a BsiWI-EcoRI fragment of about 0.41 kb.

Next, 2 μp of pKANTEX93, a vector for expression of humanized antibody, was allowed to react at 55° C. for 1 hour using 10 units of the restriction enze BsiWI (manufactured by New England Biolabs), followed by reaction at 37° C. for 1 hour using 10 units of the restriction enzyme EcoRI (manufactured by Takara Shuzo). The reaction mixture was fractionated by agarose gel electrophoresis to recover a BsiWI-EcoRI figment of about 12.75 kb.

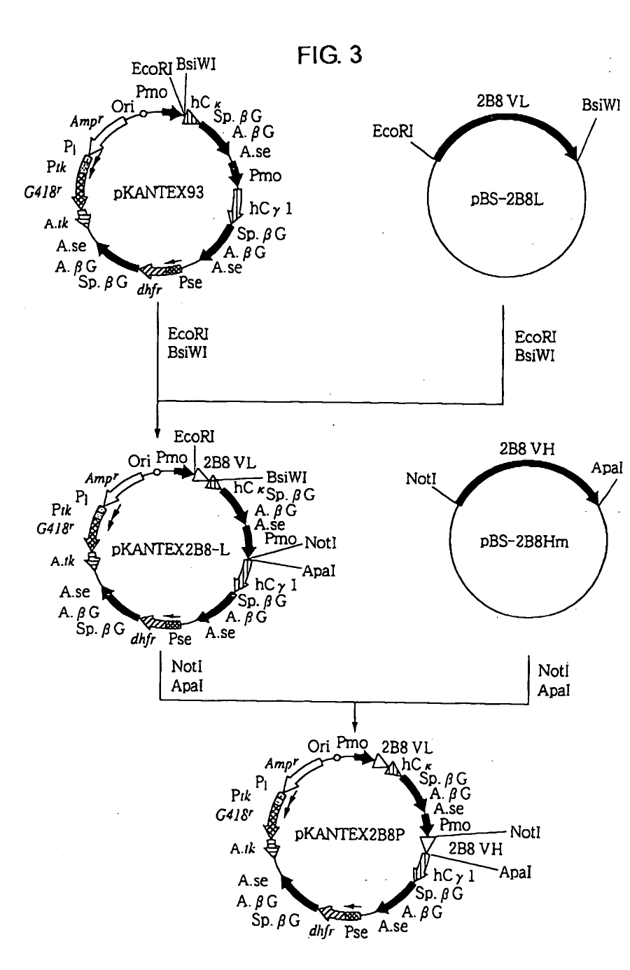

Next, the plasmid pBS-2B8L-derived BsiWI-EcoRI fragment and plasmid pKANTEX93-derived BsiWI-EcoRI fragment thus obtained were ligated by using Solution I of DNA Ligation Kit Ver, 2 (manufactured by Takara Shuzo) in accordance with the manufacture's instructions attached thereto. By using the recombinant plasmid DNA solution obtained in this manner, E. coli DH5α (manufactured by TOYOBO) was transformed to obtain the plasmid pKANTEX2B8-L shown in FIG. 3.

Next, 2 μg of the plasmid pBS-2B8Hm obtained in item 1(2) of Example 1 was allowed to react at 37° C. for 1 hour by using 10 units of a restriction enzyme ApaI (manufactured by Takara Shuzo), followed by reaction at 37° C. for 1 hour using 10 units of a restriction enzyme NotI (manufactured by Takara Shuzo). The reaction mixture was fractionated by agarose gel electrophoresis to recover an ApaI-NotI fragment of about 0.45 kb.

Next, 3 μg of the plasmid pKANTEX2B8-L was allowed to react at 37° C. for 1 hour by using 10 units of the restriction enzyme ApaI (manufactured by Takara Shuzo), followed by reaction at 37° C. for 1 hour using 10 units of the restriction enzyme NotI (manufactured by Takara Shuzo). The reaction mixture was fractionated by agarose gel electrophoresis to recover an ApaI-NotI fragment of about 13.16 kb.

Next, the plasmid pBS-2B8Hm-derived ApaI-NotI fragment and plasmid pKANTEX2B8-L-derived ApaI-NotI fragment thus obtained were ligated by using Solution I of DNA Ligation Kit Ver. 2 (manufactured by Takara Shuzo) in accordance with the manufacture's instructions attached thereto. By using the recombinant plasmid DNA solution obtained in this manner, E. coli DH5α (manufactured by TOYOBO) was transformed, and each plasmid DNA was prepared from the transformant clones.

The nucleotide sequence of the thus obtained plasmid was analyzed by using BigDye Terminator Cycle Sequencing Ready Reaction Kit v 2.0 (manufactured by Applied Biosystems) and the DNA sequencer 377 of the same company, and it was confirmed that the plasmid pKANTEX2B8P shown in FIG. 3 into which the objective DNA had been cloned was obtained.

2. Stable Expression of an Anti-CD20 Chimeric Antibody by Using Animal Cells

(1) Preparation of a Production Cell by Using Rat Myeloma YB2/0 Cell

By using the anti-CD20 chimeric antibody expression vector, pKANTEX2B8P, obtained in item 1(3) of Example 1, the anti-CD20 chimeric antibody was expressed in animal cells as follows.

After 10 μg of the plasmid pKANTEX2B8P was introduced into 4×10¹⁶ cells of a rat myeloma cell line YB2/0 cell (ATCC CRL 1662) by electroporation [Cytotechnology, 3, 133 (1990)], the cells were suspended in 40 ml of H—SFM medium (manufactured by GIBCO-BRL supplemented with 5% fetal calf serum (FCS)) and dispensed at 200 μl/well into a 96 well microtiter plate (manufactured by Sumitomo Bakelite). After culturing at 37° C. for 24 hours in a 5% CO₂ incubator, G418 was added thereto to give a concentration of 1 mg/ml, followed by culturing for 1 to 2 weeks. Culture supernatants were recovered from wells where colonies of transformants showing G418 resistance were formed and transformants became confluent, and the produced amount of the human IgG antibody in the culture supernatant was measured by ELISA described in item 2(2) of Example 1.

Regarding a transformant in a well where expression of human IgG antibody was found in the culture supernatant, in order to increase the antibody expression level using a dhfr gene amplification system, it was suspended in H—SFM medium containing 1 mg/ml G418 and 50 nM methotrexate (hereinafter referred to as “MTX”, manufactured by SIGMA) as an inhibitor of the dhfr gene product dihydrofolate reductase (hereinafter referred to as “DHFR”) to give a density of 1 to 2×10⁵ cells/ml, and the suspension was dispensed at 1 ml into each well of a 24 well plate (manufactured by Greiner). Culturing was cared out at 37° C. for 1 to 2 weeks in a 5% CO₂ incubator to induce transformants showing 50 DM MTX resistance. When a transformant became confluent in a well, the produced amount of the human IgG antibody in the culture supernatant was measured by ELISA described in item 2(2) of Example 1. Regarding a transformant in well where expression of human IgG antibody was found in the culture supernatant, the M concentration was increased to 100 nM and then to 200 nM by the same method to finally obtain a transformant which can grow in H—SFM containing 1 mg/ml of G418 and 200 nM of MTX and also can perform high expression of the anti-CD20 chimeric antibody. The obtained transformant was cloned by limiting dilution, whereby a clone KM3065 which expresses an anti-CD20 chimeric antibody was obtained. Also, using the determination method of transcription product of α1,6-fucosyltransferase gene described in Example 8 of WO 00/61739, a cell line producing a relatively low level of the transcription product was selected and used as a suitable cell line.

The obtained transformant clone KM3065 which produces the anti-CD20 chimeric antibody has been deposited on Dec. 21, 2001, as FERM 7834 in International Patent Organism Depositary, National Institute of Advanced Industrial Science and Technology (AIST Tsukuba Central 6, 1-1, Higashi 1-Chome Tsukuba-shi, Ibaraki-ken, Japan).

(2) Measurement of a Human IgG Antibody Concentration in Culture Supernatant (ELISA)

A goat anti-human IgG (H & L) antibody (manufactured by American Qualex) was diluted with a phosphate buffered saline (hereinafter referred to as “PBS”) to give a concentration of 1 μg/ml, dispensed at 50 μl/well into a 96 well ELISA plate (manufactured by Greiner) and then allowed to stand at 4° C. overnight for adhesion. After washing with PBS, 1% bovine serum albumin (hereinafter referred to as “BSA”, manufactured by AMPC)-containing PBS (hereinafter referred to as “1% BSA-PBS”) was added thereto at 100 μl/well and allowed to react at room temperature for 1 hour to block the remaining active groups. After discarding 1% BSA-PBS, culture supernatant of a transformant and variously diluted solutions of a purified human chimeric antibody were added thereto at 50 μl/well and allowed to react at room temperature for 2 hours. After the reaction, each well was washed with 0.05% Tween 20-containing PBS (hereinafter referred to as “Tween-PBS”), and then, as a secondary antibody solution, a peroxidase-labeled goat anti-human IgG (H & L) antibody solution (manufactured by American Qualex) 3,000 folds-diluted with 1% BSA-PBS was added thereto at 50 μl/well and allowed to react at room temperature for 1 hour. After the reaction and subsequent washing with Tween-PBS, an ABTS substrate solution (a solution prepared by dissolving 0.55 g of 2,2′-azino-bis(3-ethylbenzothiazoline-6-sulfonic acid)ammonium in 1 liter of 0.1 M citrate buffer (pH 4.2), and adding 1 μl/ml hydrogen peroxide just before use) was dispensed at 50 μl/well for coloration, and the absorbance at 415 nm (hereinafter referred to as “OD415”) was measured.

3. Purification of Anti-CD20 Chimeric Antibody from Culture Supernatant

The transformant cell clone KM3065 capable of expressing the anti-CD20 chimeric antibody, obtained in item 2(1) of Example 1, was suspended in H—SFM (manufactured by GIBCO-BRL) containing 200 nM MTX and 5% of Daigo's GF21 (manufactured by Wako Pure Chemical Industries), to give a density of 1×10⁵ cells/ml, and dispensed at 50 ml into 182 cm² flasks (manufactured by Greiner). The cells were cultured at 37° C. for 7 days in a 5% CO₂ incubator, and the culture supernatant was recovered when they became confluent. The anti-CD20 chimeric antibody KM3065 was purified from the culture supernatant using a Prosep-A (manufactured by Millipore) column in accordance with the manufacture's instructions attached thereto. About 3 μg of the obtained anti-CD20 chimeric antibody KM3065 was subjected to electrophoresis in accordance with the known method [Nature, 227, 680 (1970)] to examine its molecular weight and purification degree. As a result, the purified anti-CD20 chimeric antibody KM3065 was about 150 kilodaltons (hereinafter referred to as “Kd”) under non-reducing condition, and two bands of about 50 Kd and about 25 Kd were observed under reducing conditions. These sizes of protein coincided with reports stating that an IgG type antibody has a molecular weight of about 150 Kd under non-reducing condition and is degraded into H chain having a molecular weight of about 50 Kd and L chain having a molecular weight of about 25 Kd under reducing condition due to cutting of the intramolecular disulfide bond (hereinafter referred to as “S—S bond”) [Antibodies: A Laboratory Manual, Cold Spring Harbor Laboratory, Chapter 14 (1988), Monoclonal Antibodes: Principles and Practice, Academic Press Limited (1996)] and also almost coincided with the electrophoresis pattern of Rituxan™, and accordingly, it was confirmed that the anti-CD20 chimeric antibody KM3065 is expressed as the antibody molecule of a correct structure.

### EXAMPLE 2

Activity Evaluation of an Anti-CD20 Chimeric Antibody:

1. Binding Activity of an Anti-CD20 Chimeric Antibody to CD20-Expressing Cells (Immunofluorescent Method)

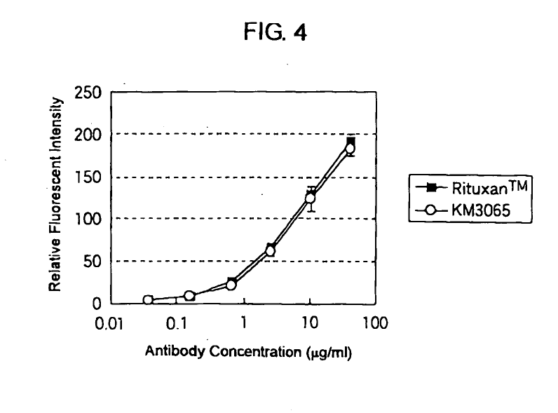

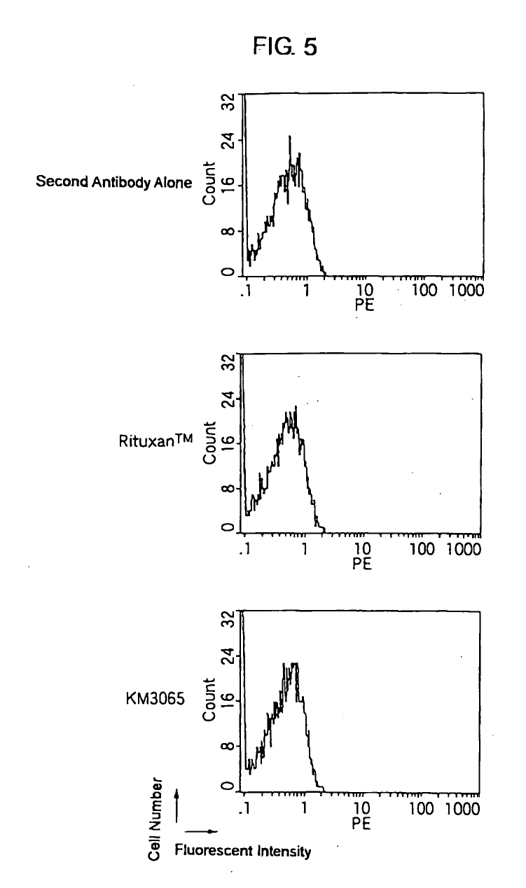

Binding activity of the purified CD20 chimeric antibody obtained in item 3 of Example 1 was evaluated by an immunofluorescent method using a flow cytometry. A human lymphoma cell line, Raji cell (JCRB 9012), as a CD20-positive cell was dispensed at of 2×10⁵ cells into each well of a 96 well U-shape plate (manufactured by Falcon). An antibody solution (a concentration of 0.039 to 40 μg/ml) prepared by diluting the anti-CD20 chimeric antibody with an FACS buffer (1% BSA-PBS, 0.02% EDTA, 0.05% NaN₃) was added thereto at 50 μl/well and allowed to react on ice for 30 minutes. After washing twice with 200 μl/well of the FACS buffer, a solution prepared by diluting a PE-labeled anti-human IgG antibody (manufactured by Coulter) 100 folds with FACS buffer was added thereto at 50 μl/well. After 30 minutes of the reaction on ice under a shade and subsequent three times of washing at 200 μl/well, the cells were finally suspended at 500 μl of the mixture to measure the fluorescence intensity by a flow cytometer. The results are shown in FIG. 4. Antibody concentration-dependent increase in the fluorescence intensity was observed in both of KM3065 and Rituxan™, and it was confirmed that they show almost the same binding activity. Also, their activity to bind to a CD20-negative cell, human CCRF—CEM cell (ATCC CCL 119), was examined in the same manner by adjusting the antibody concentration to 40 μg/ml. The results are shown in FIG. 5. Since neither K3065 nor Rituxcn™ bound thereto, it was suggested that KM065 specifically binds to CD20.

2. In Vitro Cytotoxic Activity (ADCC Activity) of an Anti-CD20 Chimeric Antibody

In order to evaluate in vitro cytotoxic activity of the purified anti-CD20 chimeric antibodies obtained in item 3 of Example 17 the ADCC activity was measured in accordance with the following method.

(1) Preparation of a Target Cell Solution

A human B lymphocyte cultured cell line WEL2-S cell (ATCC CRLS885), Ramos cell (ATCC CRL1596) or Raji cell (JCRB9012) cultured in RPW1640-FCS(10) medium (RPMI1640 medium (manufactured by GIBCO BRL) containing 10% FCS) were washed with RPMI1640-FCS(5) medium (RPMI1640 medium (manufactured by GIBCO BRL) containing 5% FCS) by centrifugation and suspension, and then adjusted to 2×10⁵ cells/ml by adding the RPMI1640-FCS(5) medium as the target cell solution.

(2) Preparation of an Effector Cell Solution

From a healthy person, 50 ml of venous blood was collected, and 0.5 ml of heparin sodium (manufactured by Shimizu Pharmaceutical) was added thereto and gently mixed. The mixture was centrifuged to isolate a mononuclear cell layer using Lymphoprep (manufactured by AXIS SHIED) in accordance with the manufacture's instructions (800×g, 20 minutes). After washing with the RPMI1640-FCS(5) medium by centrifugation three times, the resulting precipitate was re-suspended to give a density of 4×10⁶ cells/ml using the same medium and used as an effector cell solution.

(3) Measurement of ADCC Activity

Into each well of a 96 well U-shaped bottom plate (manufactured by Falcon), 50 μl of the target cell solution prepared in the above (1) (1×10⁴ cells/well) was dispensed. Next, 50 μl of the effector cell solution prepared in the above (2) was added thereto (2×10⁵ cells/well, the ratio of effector cells to target cells became 20:1). Subsequently, each of the anti-CD20 chimeric antibodies was added thereto to give a final concentration from 0.3 to 3000 ng/ml, and the total volume was made up to 150 μl, followed by reaction at 37° C. for 4 hours. After the reaction, the plate was centrifuged, and the lactate dehydrogenase (LDH) activity in the supernatant was measured by obtaining the absorbance data using CytoTox96 Non-Radioactive Cytotoxicity Assay (manufactured by Promega) according to the manufacturers instructions. The absorbance data of spontaneously released target cells and the absorbance data of spontaneously released effector cells were obtained in the same manner as the above, except that the medium alone was used instead of the effector cell solution and the antibody solution, and that the medium alone was used instead of the target cell solution and the antibody solution, respectively. The absorbance data of the total released target cells was obtained by measuring the LDH activity in the supernatant in the same manner as the above, by using the medium instead of the antibody solution and the effector cell solution and adding 15 μl of a 9% Triton X-100 solution to the medium 45 minutes before the reaction termination. The ADCC activity was measured by the following equation.

$$\begin{matrix}Cytotoxic \\ activity ( \% )\end{matrix} = \frac{\begin{matrix}( \begin{matrix}Absorbance \\ of sample\end{matrix} ) - ( \begin{matrix}Absorbance of \\ spontaneously \\ released effector cells\end{matrix} ) - \\ ( \begin{matrix}Absorbance of \\ spontaneously \\ released target cells\end{matrix} )\end{matrix}}{( \begin{matrix}Absorbance of total \\ released target cells\end{matrix} ) - ( \begin{matrix}Absorbance of \\ spontaneously \\ released target cells\end{matrix} )} \times 100$$

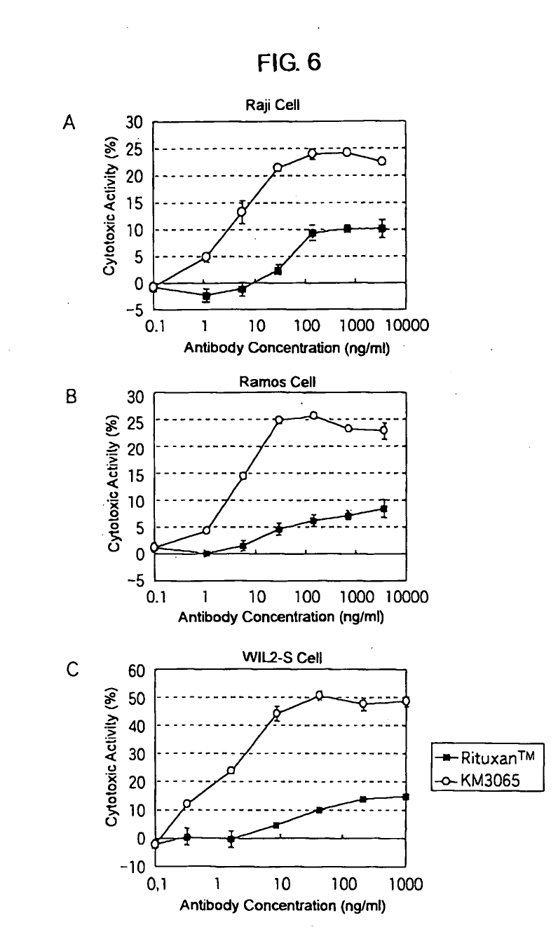

FIG. 6 shows results of using 3 cell lines as the target. FIGS. 6A, 6B and 6C show results of using Raji cell (JCRB9012), Ramos cell (ATCC CRL1596) and WIL2-S cell (ATCC CRL8885), respectively. As shown in FIG. 6, KM3065 show higher ADCC activity at all antibody concentrations and higher maximum cytotoxic activity than Rituxan™.

### EXAMPLE 3

Sugar Chain Analysis of Anti-CD20 Chimeric Antibodies:

Sugar chains of the anti-CD20 antibodies purified in item 3 of Example 1 were analyzed. The sugar chains were cleaved from proteins by subjecting KM3065 and Rituxan™ to hydrazinolysis [Method of Enzymology, 13, 263 (1982)]. After removing hydrazine by evaporation under a reduced pressure, N-acetylation was carried out by adding an aqueous ammonium acetate solution and acetic anhydride. After freeze-drying, fluorescence labeling by 2-aminopyridine was carried out [Journal of Biochemistry, 9, 197 (1984)]. A fluorescence-labeled sugar chain group (hereinafter “PA-treated sugar chain group”) was separated from excess reagents using Superdex Peptide HR 10/30 column (manufactured by Pharmacia). The sugar chain fractions were dried using a centrifugation concentrator and used as a purified PA-treated sugar chain group. Next, the purified PA-treated sugar chain group was subjected to reverse phase HPLC analysis using a CLC-ODS column (manufactured by Shimadzu).

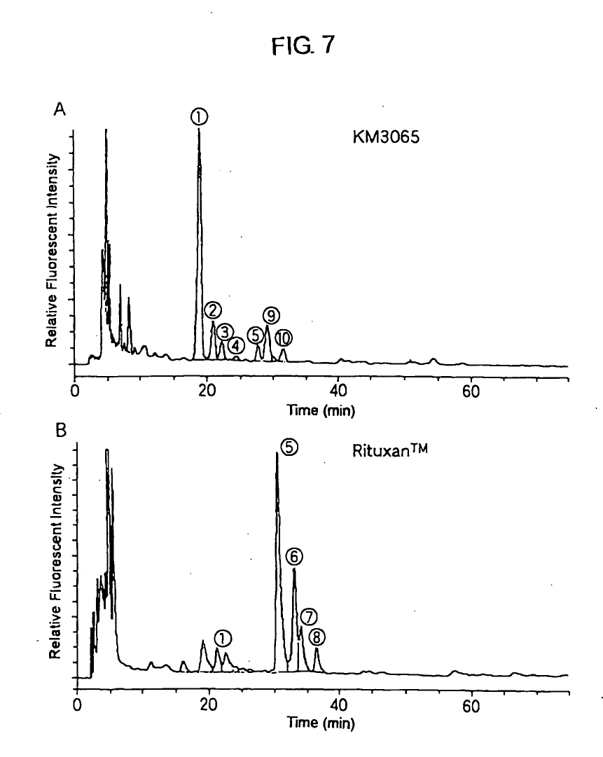

FIG. 7 shows elution patterns obtained by carrying out reverse phase HPLC analysis of each of PA-treated sugar chains prepared from the anti-CD20 chimeric antibodies. FIGS. 7A and 7B show elution patterns of KM3065 and Rituxan™, respectively. The ordinate and the abscissa show the relative fluorescence intensity and the elution time, respectively. Using a 10 mM sodium phosphate buffer (pH 3.8) as buffer A and a 10 mM sodium phosphate buffer (pH 3.8)+0.5% 1-butanol as buffer B, the analysis was carried out by the following gradient.

TABLE 1

| | | | | | |
|---|---|---|---|---|---|
| Time (minutes) | 0 | 80 | 90 | 90.1 | 120 |
| Buffer B (%) | 0 | 60 | 60 | 0 | 0 |

Peaks \mathring{1} to \mathring{10} in FIG. 7 show the following structures.

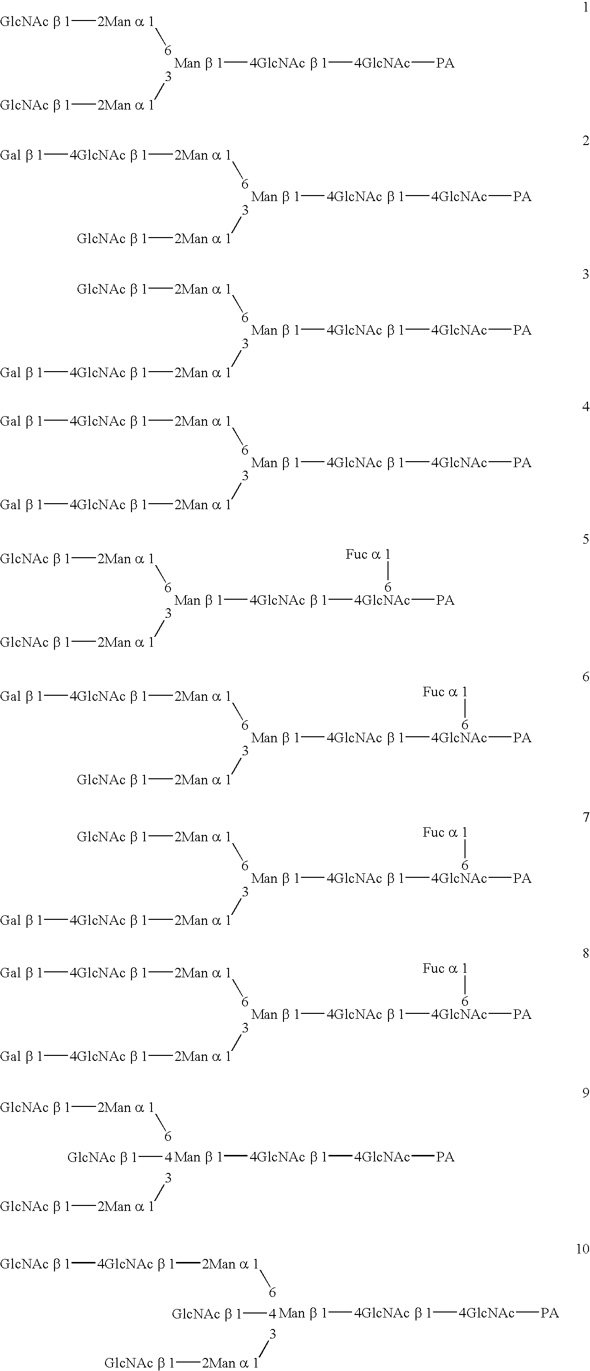

GlcNAc, Gal, Man, Fuc and PA represent N-acetylglucosamine, galactose, mannose, fucose and a pyridylamino group, respectively. In FIG. 7, the ratio of the sugar chain group in which 1-position of fucose is not bound to 6-position of N-acetylglucosamine in the complex N-glycoside-inked reducing end through α-bond (hereinafter referred to as “α1,6-fucose-free sugar chain group” or “α1,6-fucose-not-bound sugar chain group”) was calculated from the area occupied by the peaks \mathring{1} to \mathring{4}, \mathring{9} and \mathring{10} among the areas occupied by the peaks \mathring{1} to \mathring{10}. Also, the ratio of the sugar chain group in which 1-position of fucose is bound to 6-position of N-acetylglucosamine in the complex N-glycoside-linked reducing end through α-bond (hereinafter referred to as “α1,6-fucose-bound sugar chain group”) was calculated from the area occupied by the peaks \mathring{5} to \mathring{8} among the areas occupied by the peaks of \mathring{1} to \mathring{10}.

As a result, in Rituxan™, the ratio of the α1,6-fucose-not-bound sugar chains was 6%, whereas the ratio of the α1,6-fucose-bound sugar chains was 94%. In KM3065, the ratio of the α1,6-fucose-not-bound sugar chains was 96%, whereas the ratio of the α1,6-fucose-bound sugar chains was 4%. The results show that KM3065 has a much higher ratio of the α1,6-fucose-not-bound sugar chains than Rituxan™.

### EXAMPLE 4

Preparation of α1,6-fucosyltransferase (FUT8) Gene Derived from CHO Cell:

(1) Preparation of α1,6-fucosyltransferase (FUT8) cDNA Sequence from CHO Cell

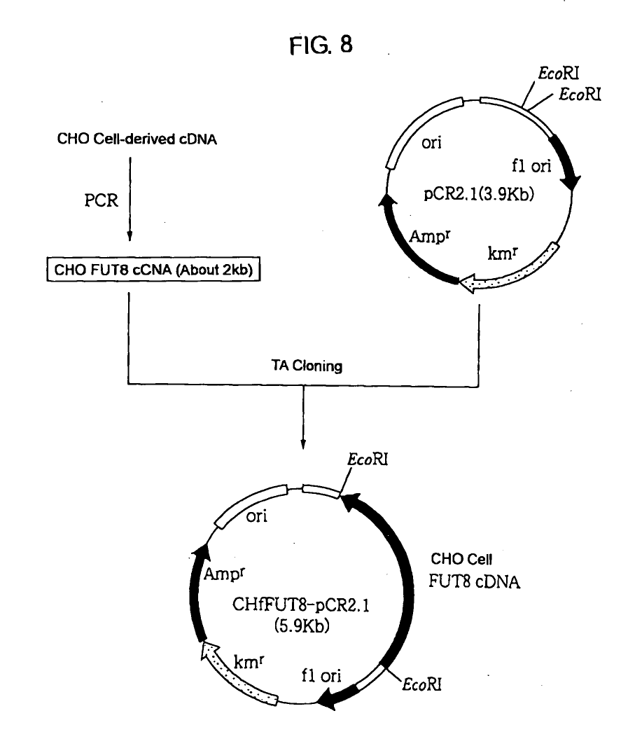

From a single-stranded cDNA prepared from CHO/DG44 cells on the 2nd day of culturing in Example 8(1) of WO00/61739, Chinese hamster FUT8 cDNA was obtained by the following procedure (FIG. 8).

First, a forward primer specific for a 5′-terminal non-translation region (shown in SEQ ID NO:21) and a reverse primer specific for a 3′-terminal non-translation region (shown in SEQ ID NO:22) were designed from a mouse FUT8 cDNA sequence (GeBank, AB025198).

Next, 25 μl of a reaction mixture [ExTaq buffer (manufactured by Takara Shuzo), 0.2 mmol/l dNTPs, 4% DMSO and 0.5 μmol/l specific primers (SEQ ID NOs:21 and 22)) containing 1 μl of the CHO/DG44 cell-derived cDNA was prepared and PCR was carried out by using a DNA polymerase ExTaq (manufactured by Takara Shuzo). The PCR was carried out by heating at 94° C. for 1 minute, subsequent 30 cycles of heating at 94° C. for 30 seconds, 55° C. for 30 seconds and 72° C. for 2 minutes as one cycle, and final heating at 72° C. for 10 minutes.

After the PCR, the reaction mixture was subjected to 0.8% agarose gel electrophoresis, and a specific amplified fragment of about 2 Kb was purified. Into a plasmid pCR2.1, 4 μl of the DNA fragment was introduced in accordance with the manufacture's instructions attached to TOPO TA Cloning Kit (manufactured by Invitrogen), and E. coli DH5α: was transformed with the reaction mixture. Plasmid DNAs were isolated from cDNA-inserted 8 clones among the obtained kanamycin-resistant colonies in accordance with a known method.

The nucleotide sequence of each cDNA inserted into the plasmid was determined using DNA Sequencer 377 (manufactured by Applied Biosystems) and BigDye Terminator Cycle Sequencing FS Ready Reaction Kit (manufactured by Applied Biosystems) in accordance with the method of the manufacture's instructions. It was confirmed by the method that all of the inserted cDNAs encode a sequence containing the fill ORF of CHO cell FUT8. Among these, a plasmid DNA containing absolutely no reading error of bases by the PCR in the sequences was selected. Herein, the plasmid is referred to as CHfFUT8-pCR2.1. The determined nucleotide sequence of the cDNA of CHO FUT8 is represented by SEQ ID NO:1. The translation region (open reading frame: ORF) in SEQ ID NO:1 is nucleotides at position 100-1827, and the amino acid sequence corresponding to nucleotides at positions 100 to 1824 excluding the termination codon is represented by SEQ ID NO-23.

(2) Preparation of α1,6-fucosyltransferase (FUT8) Genomic Sequence from CHO Cell

Using the ORF full length cDNA fragment of CHO cell FUT8 obtained in item (1) as a probe, a CHO cell FUT8 genomic clone was obtained from CHO-K1 cell-derived λ-phage genome library (manufactured by Strategene) in accordance with a known genome screening method described, e.g., in Molecular Cloning, Second Edition, Current Protocols in Molecular Biology, A Laboratory Manual, Second Edition (1989). Next, after digesting the obtained genomic clone using various restriction enzymes, the Southern hybridization was carried out by using an AfaI-Sau3AI fragment (about 280 bp) containing initiation codon of the CHO cell FUT8 cDNA as a probe, and then a XbaI-XbaI fragment (about 2.5 Kb) and a SacI-SacI fragment (about 6.5 Kb) were selected from restriction enzyme fragments showing positive reaction, inserted into pBluescript II KS(+) (manufactured by Stratagene), respectively.

The nucleotide sequence of each of the obtained genomic fragments was determined by using DNA Sequencer 377 (manufactured by Applied Biosystems) and BigDye Terminator Cycle Sequencing FS Ready Reaction Kit (manufactured by Parkin Elmer) in accordance with the method of the manufacture's instructions. Thereby, it was confirmed that the XbaI-XbaI fragment encodes a sequence of an upstream intron of about 2.5 Kb containing exon 2 of the CHO cell FUT8, and the SacI-SacI fragment encodes a sequence of a downstream intron of about 6.5 Kb containing exon 2 of the CHO cell FUT8. Herein, the plasmid containing XbaI-XbaI fragment and the plasmid containing SacI-SacI fragment are referred to as pFUT8fgE2-2 and pFUT8fgE2-4, respectively. The determined nucleotide sequence (about 9.0 Kb) of the genome region containing exon 2 of the CHO cell FUT8 is shown in SEQ ID NO:3.

### EXAMPLE 5

Preparation of CHO Cell in Which α1,6-fucosyltransferase Gene is Disrupted:

A CHO cell from which the genomic region comprising exon 2 of α1,6-fucosyltransferase FUT8) gene derived from the CHO cell was deleted was prepared and the ADCC activity of an antibody produced by the cell was evaluated.

1. Construction of Chinese Hamster α1,6-fucosyltransferase (FUT8) Gene Exon 2 Targeting Vector Plasmid pKOFUT8Puro

(1) Construction of Plasmid ploxPPuro

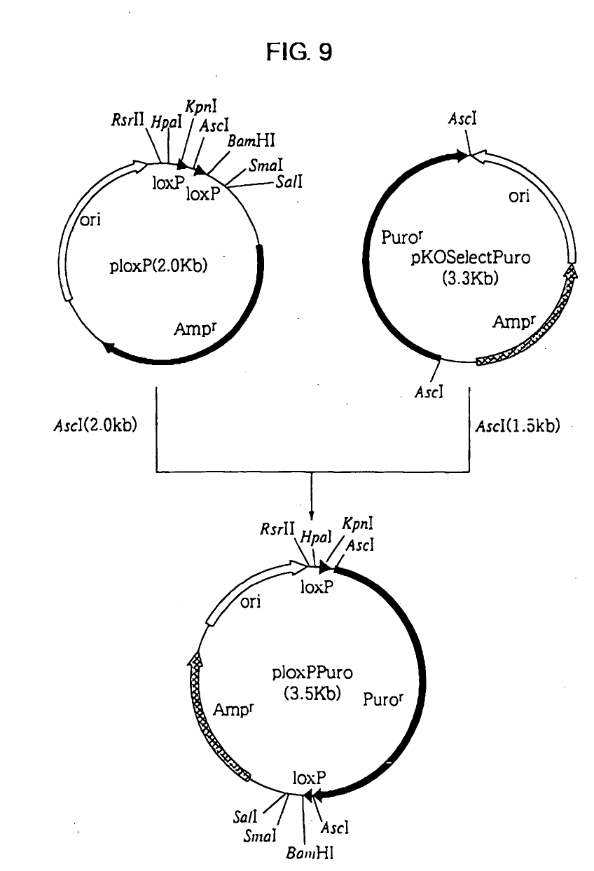

A plasmid ploxPPuro was constructed by the following procedure (FIG. 9).

In 35 μl of NEBuffer 4 (manufactured by New England Biolabs), 1.0 μg of a plasmid pKOSelectPuro (manufactured by Lexicon) was dissolved, and 20 units of a restriction enzyme AscI (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 1.5 Kb containing a puromycin resistance gene expression unit.

Separately, 1.0 μg of a plasmid ploxP described in Japanese Published Examined Patent Application No. 314512/99 was dissolved in 35 μl of NEBuffer 4 (manufactured by New England Biolabs), and 20 units of a restriction enzyme AscI (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 2.0 Kb.

The obtained AscI-AscI fragment (45 μl, about 1.5 Kb) derived from the plasmid pKOSelectPuro, 0.5 μl of the AscI-AscI fragment (about 2.0 Kb) derived from the plasmid ploxP and 5.0 μl of Ligation High (manufactured by Toyobo) were mixed, followed by ligation reaction at 16° C. for 30 minutes. E. coli DH5α was transformed by using the reaction mixture, and a plasmid DNA was isolated in accordance with a known method from the obtained ampicillin-resistant clones. Herein, the plasmid is referred to as ploxPPuro.

(2) Construction of Plasmid pKOFUT8 gE2-1

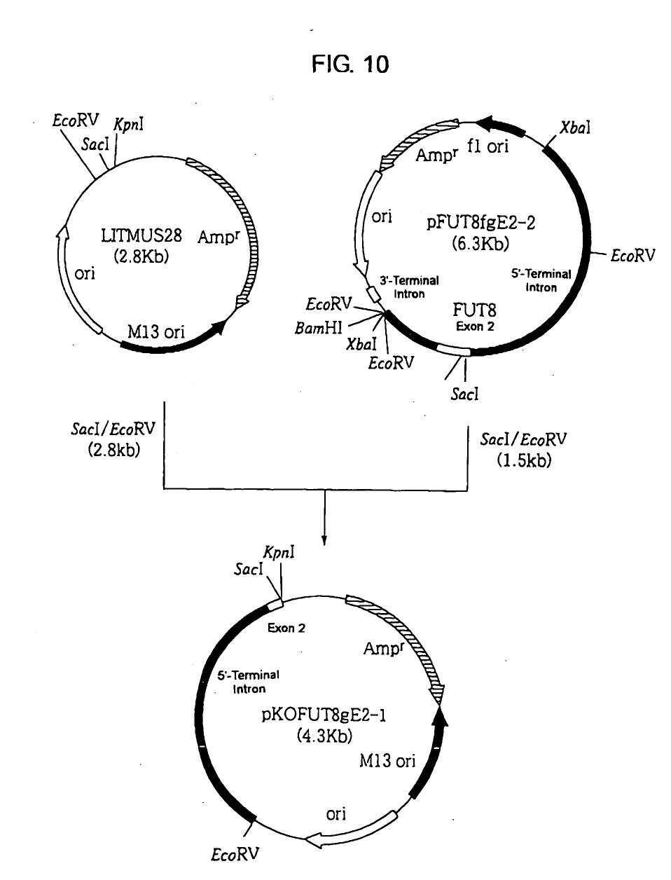

A plasmid pKOFUT8 g2-1 was constructed by the following procedure, by using the plasmid pFUT8fgE2-2 having a genome region comprising exon 2 of Chinese hamster FUT8 obtained in Example 4(2) (FIG. 10).

In 35 μl of NEBuffer 1 (manufactured by New England Biolabs) containing 100 μg/ml of BSA (manufactured by New England Biolabs), 2.0 μg of the plasmid pFUT8fgE2-2 was dissolved, and 20 units of a restriction enzyme SacI (manufactured by New England Biolabs) were added thereto, followed by digestion reaction at 37° C. for 2 hours. A DNA fragment was recovered from the reaction mixture by ethanol precipitation and dissolved in 35 μl of NEBuffer 2 (manufactured by New England Biolabs) containing 100 μg/ml BSA (manufactured by New England Biolabs), and 20 units of a restriction enzyme EcoRV (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 1.5 Kb.

Separately, 1.0 μg of a plasmid LITMUS28 (manufactured by New England Biolabs) was dissolved in 35 μl of NEBuffer I (manufactured by New England Biolabs) containing 100 μg/ml of BSA (manufactured by New England Biolabs), and 20 units of a restriction enzyme SacI (manufactured by New England Biolabs) were added thereto, followed by digestion reaction at 37° C. for 2 hours. A DNA fragment was recovered from the reaction mixture by ethanol precipitation and dissolved in 35 μl of NEBuffer 2 (manufactured by New England Biolabs) containing 100 μg/ml BSA (manufactured by New England Biolabs), and 20 units of a restriction enzyme EcoRV (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 2.8 Kb.

The obtained EcoRV-SacI fragment (4.5 μl, about 1.5 Kb) derived from the plasmid pFUT8fgE2-2, 0.5 μl of the EcoRV-SacI fragment (about 2.8 Kb) derived from the plasmid LITMUS28 and 5.0 μl of Ligation High (manufactured by Toyobo) were mixed, followed by ligation reaction at 16° C. for 30 minutes. E coli DH5α was transformed by using the reaction mixture, and a plasmid DNA was isolated from the obtained ampicillin-resistant clones in accordance with a known method. Herein, the plasmid is referred to as pKOFUT8gE2-1.

(3) Construction of Plasmid pKOFUT8gE2-2

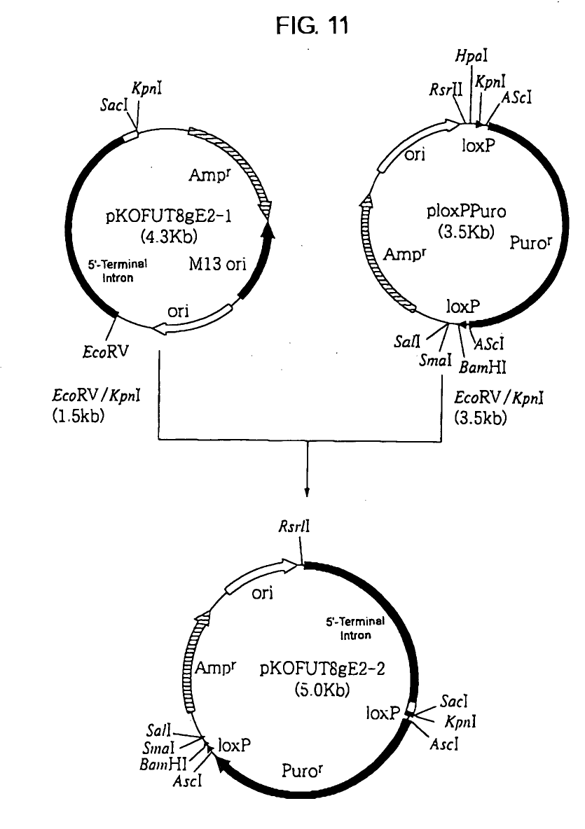

A plasmid pKOFUT8gE2-2 was constructed by the following procedure, by using the plasmid pKOFUT8gE2-1 obtained in item (2) (FIG. 11).

In 30 μl of NEBuffer 2 (manufactured by New England Biolabs) containing 100 μg/nm of BSA (manufactured by New England Biolabs), 2.0 μg of the plasmid pKOFUT8gE2-1 was dissolved, and 20 units of a restriction enzyme EcoRV (manufactured by New England Biolabs) were added thereto, followed by digestion reaction at 37° C. for 2 hours. A DNA fragment was recovered from the reaction mixture by ethanol precipitation and dissolved in 30 μl of NEBuffer 1 (manufactured by New England Biolabs) containing 100 μg/ml BSA (manufactured by New England Biolabs), and 20 units of a restriction enzyme KpnI (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 1.5 Kb.

Separately, 1.0 μg of the plasmid ploxPPuro was dissolved in 30 μl of NEBuffer 4 (manufactured by New England Biolabs), and 20 units of a restriction enzyme HpaI (manufactured by New England Biolabs) were added thereto, followed by digestion reaction at 37° C. for 2 hours. A DNA fragment was recovered from the reaction mixture by ethanol precipitation and dissolved in 30 μl of NEBuffer 1 (manufactured by New England Biolabs) containing 100 μg/mil BSA (manufactured by New England Biolabs), and 20 units of a restriction enzyme KpnI (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 3.5 Kb.

The obtained EcoRV-KpnI fragment (4.0 μl, about 1.5 Kb) derived from the plasmid pKOFUT8gE2-1, 1.0 μl of the HpaI-KpnI fragment (about 3.5 Kb) derived from the plasmid ploxPPuro and 5.0 μl of Ligation High (manufactured by Toyobo) were mixed, followed by ligation reaction at 16° C. for 30 minutes. E. coli DH5α was transformed by using the reaction mixture, and a plasmid DNA was isolated in accordance with a known method from the obtained ampicillin-resistant clones. Herein, the plasmid is referred to as pKOFUT8gE2-2.

(4) Construction of Plasmid pscFUT8gE2-3

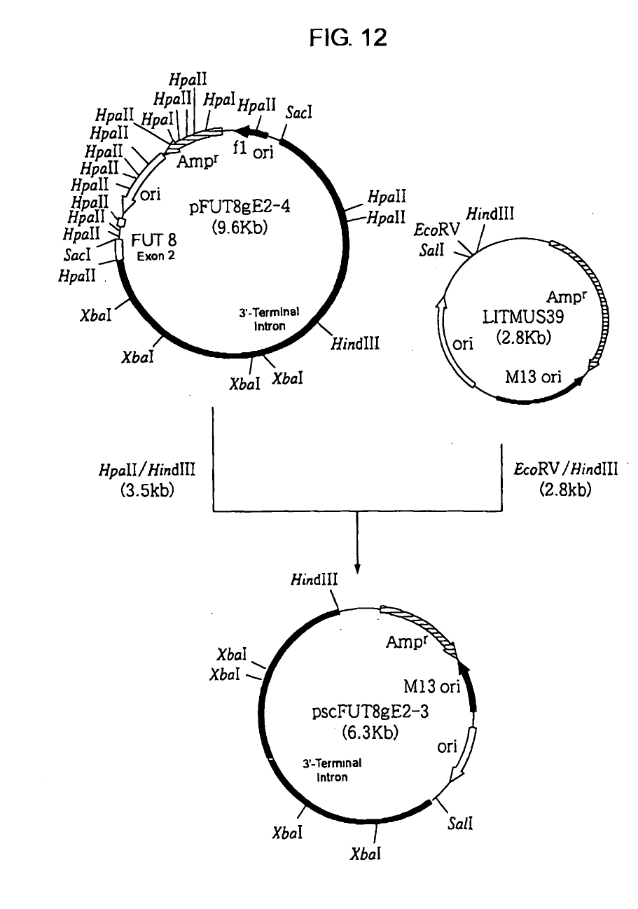

A plasmid pscFUT8gE2-3 was constructed by the following procedure, by using the plasmid pFUT8fgE24 having a genome region comprising exon 2 of Chinese hamster FUT8 obtained in Example 4(2) (FIG. 12).

In 35 μl of NEBuffer 1 (manufactured by New England Biolabs), 2.0 μg of the plasmid pFUT8fgE2-4 was dissolved, and 20 units of a restriction enzyme HpaII (manufactured by New England Biolabs) were added thereto, followed by digestion reaction at 37° C. for 2 hours. A DNA fragment was recovered from the reaction mixture by ethanol precipitation, and then the DNA termini were changed to blunt ends by using Blunting High (manufactured by Toyobo) in accordance with the manufacture's instructions. The DNA fragment was recovered by carrying out phenol/chloroform extraction and ethanol precipitation and dissolved in 35 μl of NEBuffer 2 (manufactured by New England Biolabs), and 20 units of a restriction enzyme HindIII (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 3.5 Kb.

Separately, 1.0 μg of a plasmid LITMUS39 (manufactured by New England Biolabs) was dissolved in 35 μl of NEBuffer 2 (manufactured by New England Biolabs), and the mixture was mixed with 20 units of a restriction enzyme EcoRV (manufactured by New England Biolabs) and 20 units of a restriction enzyme HindIII (manufactured by New England Biolabs) and subjected to the digestion reaction at 37° C. for 2 hours. After the digestion reaction, the mixture was subjected to 0.8% (w/v) agarose gel electrophoresis to purify a DNA fragment of about 2.8 Kb.

The obtained HpaII-HindIII fragment (4.0 μl, about 3.5 Kb) derived from the plasmid pFUT8fgE24, 1.0 μl of the EcoRV-HindIII fragment (about 2.8 Kb) derived from the plasmid LITMUS39 and 5.0 μl of Ligation High (manufactured by Toyobo) were mixed, followed by ligation reaction at 16° C. for 30 minutes. E. coli DH5α was transformed by using the reaction mixture, and a plasmid DNA was isolated in accordance with a known method from the obtained ampicillin-resistant clones. Herein, the plasmid is referred to as pscFUT8gE2-3.

(5) Construction of Plasmid pKOFUT8gE2-3

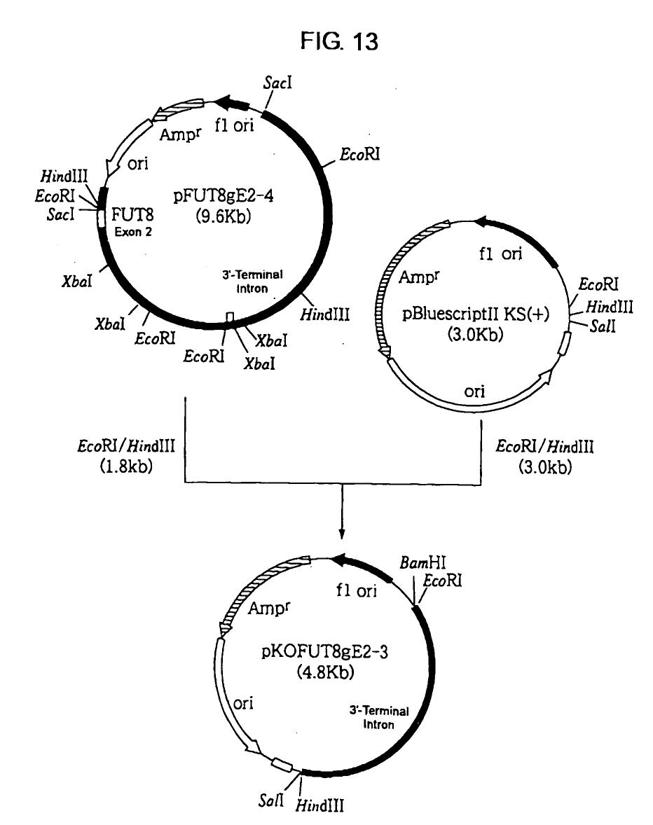

A plasmid pKOFUT8gE2-3 was constructed by the following procedure, by using the plasmid pFUT8fgE2-4 obtained in Example 4(2) having a genome region comprising exon 2 of Chinese hamster FUT8 (FIG. 13).

In 35 μl of NEBuffer for EcoRI (manufactured by New England Biolabs), 2.0 μg of the plasmid pFUT8fgE2-4 was dissolved, and 20 units of a restriction enzyme EcoRI (manufactured by New England Biolabs) and 20 units of a restriction enzyme HindIII (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 1.8 Kb.

Separately, 1.0 μg of a plasmid pBluescript II KS(+) (manufactured by Stratagene) was dissolved in 35 μl of NEBuffer for EcoRI (manufactured by New England Biolabs). Then 20 units of a restriction enzyme EcoRI (manufactured by New England Biolabs) and 20 units of a restriction enzyme HindIII (manufactured by New England Biolabs) were added thereto, followed by digestion reaction at 37° C. for 2 hours. After the digestion reaction, the mixture was subjected to 08% (w/v) agarose gel electrophoresis to purify a DNA fragment of about 3.0 Kb.

The obtained HindIII-EcoRI fragment (4.0 pd, about 1.8 Kb) derived from the plasmid pFUT8fgE2-4, 1.0 μl of the HindIII-EcoRI fragment (about 3.0 Kb) derived from the plasmid pBluescript II KS(+) and 5.0 μl of Ligation High (manufactured by Toyobo) were mixed, followed by ligation reaction at 16° C. for 30 minutes. E. coli DH5α was transformed by using the reaction mixture, and a plasmid DNA was isolated in accordance with a known method from the obtained ampicillin-resistant clones. Herein, the plasmid is referred to as pKOFUT8gE2-3.

(6) Construction of Plasmid pKOFUT8gE2-4

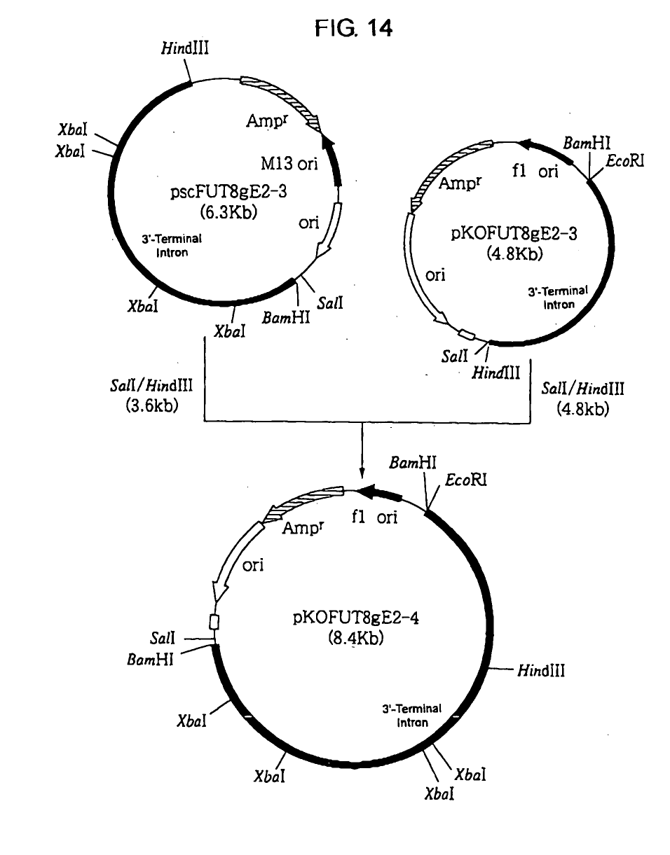

A plasmid pKOFUT8gE2-4 was constructed by the following procedure, by using the plasmids pscFUT8gE2-3 and pKOFUT8gE2-3 obtained in items (4) and (5) (FIG. 14).

In 35 μl of NEBuffer for SalI (manufactured by New England Biolabs) containing 100 μg/ml of BSA (manufactured by New England Biolabs), 1.0 μg of the plasmid pscFUT8gE2-3 was dissolved, and 20 units of a restriction enzyme SalI (manufactured by New England Biolabs) were added thereto, followed by digestion reaction at 37° C. for 2 hours. A DNA fragment was recovered from the reaction mixture by ethanol precipitation and dissolved in 30 μl of NEBuffer 2 (manufactured by New England Biolabs), containing 100 μg/ml BSA (manufactured by New England Biolabs), and 20 units of a restriction enzyme HindIII (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 3.6 Kb.

Separately, 1.0 μg of the plasmid pKOFUT8gE2-3 was dissolved in 35 μl of NEBuffer for SalI (manufactured by New England Biolabs), containing 100 μg/ml BSA (manufactured by New England Biolabs), and 20 units of a restriction enzyme SalI (manufactured by New England Biolabs) were added thereto, followed by digestion reaction at 37° C. for 2 hours. A DNA fragment was recovered from the reaction mixture by ethanol precipitation and dissolved in 35 μl of NEBuffer 2 (manufactured by New England Biolabs), and 20 units of a restriction enzyme HindIII (manufactured by New England Biolabs) were added thereto, followed by digestion reaction at 37° C. for 2 hours. After the digestion reaction, 35 μl of 1 mol/l Tris-HCl buffer (pH 8.0) and 3.5 μl of E. coli C15-derived alkaline phosphatase (manufactured by Takara Shuzo) were added thereto, followed by reaction at 65° C. for 30 minutes to dephosphorylate the DNA termini. After the dephosphorylation treatment, a DNA fragment was recovered by carrying out phenol/chloroform extraction and ethanol precipitation, and dissolved in 10 μl of sterile water.

The obtained SalI-HindIII fragment (4.0 μl, about 3.1 Kb) derived from the plasmid pscFUT8gE2-3, 1.0 μl of the SalI-HindIII fragment (about 4.8 Kb) derived from the plasmid pKOFUT8gE2-3 and 5.0 μl of Ligation High (manufactured by Toyobo) were mixed, followed by ligation reaction at 16° C. for 30 minutes. E coli DH5α was transformed by using the reaction mixture, and a plasmid DNA was isolated in accordance with a known method from the obtained ampicillin-resistant clones. Herein, the plasmid is referred to as pKOFUT8gE2-4.

(7) Construction of Plasmid pKOFUT8gE2-5

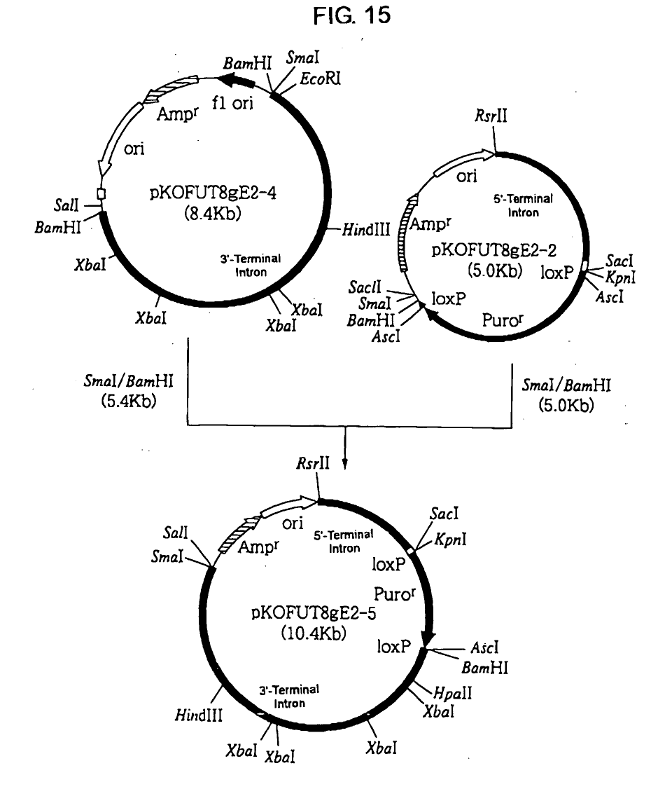

A plasmid pKOFUT8gE2-5 was constructed by the following procedure, by using the plasmids pKOFUT8gE2-2 and pKOFUT8gE2-4 obtained in items (3) and (6) (FIG. 15).

In 30 μl of NEBuffer 4 (manufactured by New England Biolabs), 1.0 μg of the plasmid pKOFUT8gE2-2 was dissolved, and 20 units of a restriction enzyme SmaI (manufactured by New England Biolabs) were added thereto, followed by digestion reaction at 25° C. for 2 hours. A DNA fragment was recovered from the reaction mixture by ethanol precipitation and dissolved in 30 μl of NEBuffer 2 (manufactured by New England Biolabs), and 20 units of a restriction enzyme BamHI (manufactured by New England Biolabs) were added thereto, followed by digestion reaction at 37° C. for 2 hours. After the digestion reaction, 30 μl of 1 mol/l Tris-HCl buffer (pH 8.0) and 3.0 μl of E. coli C15-derived alkaline phosphatase (manufactured by Takara Shuzo) were added thereto, followed by reaction at 65° C. for 1 hour to dephosphorylate the DNA termini. After the dephosphorylation treatment, the DNA fragment was recovered by carrying out phenol/chloroform extraction and ethanol precipitation, and dissolved in 10 μl of sterile water.

Separately, 1.0 μg of the plasmid pKOFUT8gE2-4 was dissolved in 30 μl of NEBuffer 4 (manufactured by New England Biolabs), and 20 units of a restriction enzyme SmaI (manufactured by New England Biolabs) were added thereto, followed by digestion reaction at 25° C. for 2 hours. A DNA fragment was recovered from the reaction mixture by ethanol precipitation and dissolved in 30 W of NEBuffer 2 (manufactured by New England Biolabs), and 20 units of a restriction enzyme BamHI (manufactured by New England Biolabs) were added thereto followed by digestion reaction at 37° C. for 2 hours. After the digestion reaction, the mixture was subjected to 0.8% (w/v) agarose gel electrophoresis to purify a DNA fragment of about 5.2 Kb.

The obtained SmaI-BamHI fragment (0.5 μl, about 5.0 Kb) derived from the plasmid pKOFUT8gE2-2, 4.5 μl of the SmaI-BamHI fragment (about 5.2 Kb) derived from the plasmid pKOFUT8gE2-4 and 5.0 μl of Ligation High (manufactured by Toyobo) were mixed, followed by ligation reaction at 16° C. for 15 hours. E. coli DH5α was transformed by using the reaction mixture, and a plasmid DNA was isolated in accordance with a known method from the obtained ampicillin-resistant clones. Herein, the plasmid is referred to as pKOFUT8gE2-5.

(8) Construction of Plasmid pKOFUT8Puro

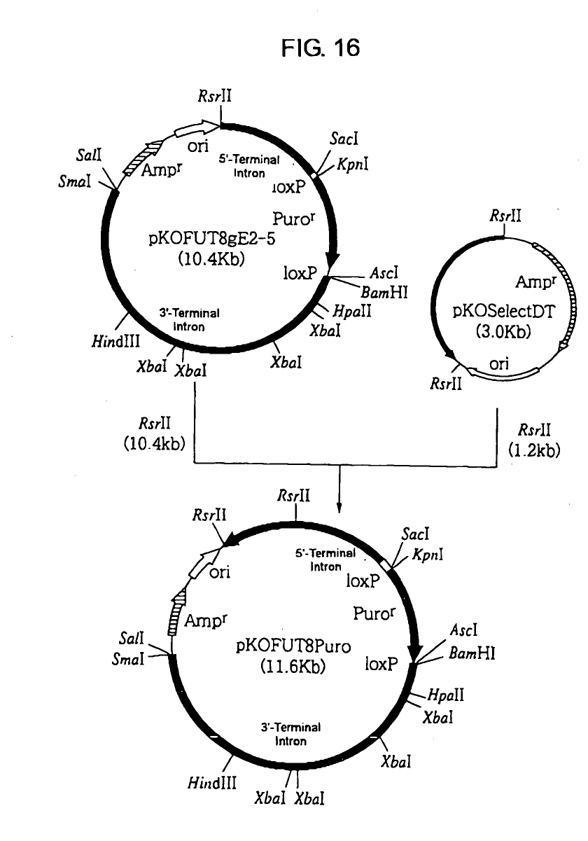

A plasmid pKOFUT8Puro was constructed by the following procedure, by using the plasmid pKOFUT8gE2-5 obtained in item (7) (FIG. 16).

In 50 μl of NEBuffer 4 (manufactured by New England Biolabs), 1.0 μg of a plasmid pKOSelectDT (manufactured by Lexicon) was dissolved, and 16 units of a restriction enzyme RsrII (manufactured by New England Biolabs) were added thereto, followed by digestion reaction at 37° C. for 2 hours. After the digestion reaction, the mixture was subjected to 0.8% (w/v) agarose gel electrophoresis to purify a DNA fragment of about 1.2 Kb comprising a diphtheria toxin expression unit.

Separately, 1.0 μg of the plasmid pKOFUT8gE2-5 was dissolved in 50 μl of NEBuffer 4 (manufactured by New England Biolabs), and 16 units of a restriction enzyme RsrII (manufactured by New England Biolabs) were added thereto, followed by digestion reaction at 37° C. for 2 hours. After the digestion reaction, 30 μl of 1 mol/l Tris-HCl buffer (pH 8.0) and 3.0 μl of E coli C15-derived alkaline phosphatase (manufactured by Takara Shuzo) were added thereto, followed by reaction at 65° C. for 1 hour to dephosphorylate the DNA termini. After the dephosphorylation treatment, the DNA fragment was recovered by carrying out phenol/chloroform extraction and ethanol precipitation, and dissolved in 10 μl of sterile water.

1.0 μg of the obtained RsrII-RsrII fragment (about 1.2 Kb) derived from the plasmid pKOSelectDT, 1.0 μl of the RsrII-RsrII fragment (about 10.4 Kb) derived from the plasmid pKOFUT8gE2-5, 3.0 μl of sterile water and 5.0 μl of Ligation High (manufactured by Toyobo) were mixed, followed by ligation reaction at 16° C. for 30 minutes. E. coli DH5α was transformed by using the reaction mixture, and a plasmid DNA was isolated in accordance with a known method from the obtained ampicillin-resistant clones. Herein, the plasmid is referred to as pKOFUT8Puro. The plasmid is used as a targeting vector for constructing CHO cell-derived FUT8 gene knock out cell.

### EXAMPLE 6

Preparation of Lectin-Resistant CHO/DG44 Cell and Production of Antibody Using the Cell:

1. Preparation of Lectin-Resistant CHO/DG44

CHO/DG44 cells were grown until they reached a stage of just before confluent, by culturing in a 75 cm² flask for adhesion culture (manufactured by Greiner) using IMDM-FBS(10) medium MOM medium comprising 10% of fetal bovine serum (FBS) and 1× concentration of HT supplement (manufactured by GIBCO BPL)]. After washing the cells with 5 ml of Dulbecco's PBS (manufactured by Invitrogen), 1.5 ml of 0.05% trypsin (manufactured by Invitrogen) diluted with Dulbecco's PBS was added thereto and allowed to stand at 37° C. for 5 minutes to dissociate the cells from the flask bottom. The disociated cells were recovered by a centrifugation operation generally used in cell culture and suspended in lMDM-EBS(10) medium at a density of 1×10⁵ cells/ml. To the cell suspension, and then 0.1 μg/ml of an alkylating agent N-methyl-N′-nitro-N-nitrosoguanidine (hereinafter referred to as “MNNG”, manufactured by Sigma) may be added, if necessary. After incubating them at 37° C. for 3 days in a CO₂ incubator (manufactured by TABAI), the culture supernatant was discarded, and the cells were again washed, dissociated and recovered by the same operations, suspended in IMDM-FBS(10) medium and then inoculated into a tissue culture 96 well plate (manufactured by IWAKI Glass) at a density of 1,000 cells/well. To each well, as the final concentration in medium, 1 mg/ml Lens culinaris agglutinin (hereinafter referred to as “LCA”, manufactured by Vector), 1 mg/ml Aleuria aurantia agglutinin (Aleuria aurantia lectin; hereinafter referred to as “AAL”, manufactured by Vector) or 1 mg/ml kidney bean agglutinin (Phaseolus vulgaris leucoagglutinin; hereinafter referred to as “L-PHA”, manufactured by Vector) was added. After culturing them at 37° C. for 2 weeks in a CO₂ incubator, the appeared colonies were obtained as lectin-resistant CHO/DG44. Regarding the obtained lectin-resistant CHO/DG44, an LCA-resistant cell line, an AAL-resistant cell line and an L-PHA-resistant cell line were named CHO-LCA, CHO-AAL and CHO—PHA, respectively. When the resistance of these cell lines to various kinds of lectin was examined, it was found that the CHO-LCA was also resistant to AAL, and the CHO-AAL was also resistant LCA. In addition, the CHO-LCA and CHO-AAL also showed a resistance to a lectin which recognizes a sugar chain structure identical to the sugar chain structure recognized by LCA and AAL, namely a lectin which recognizes a sugar chain structure in which 1-position of fucose is bound to 6-position of N-acetylglucosamine residue in the reducing end through α-bond in the N-glycoside-linked sugar chain. Specifically, it was found that the CHO-LCA and CHO-AAL can show resistance and survive even in a medium supplemented with a pea agglutinin (Pisum sativum agglutinin; hereinafter referred to as “PSA”, manufactured by Vector) at a final concentration of 1 mg/ml. In addition, even when the alkylating agent MNNG was not added, it was able to obtain lectin-resistant cell lines by increasing the number of cells to be treated. Hereinafter, these cell lines were used in analyses.

2. Preparation of anti-CD20 Human Chimeric Antibody-Producing Cells

Into 1.6×10⁶ cells of the CHO/DG44 cell which was the lectin-resistant cell line obtained in the above item 1, 4 μg of an anti-CD20 vector for expression of human chimeric antibody pKANTEX2B8P was introduced by electroporation [Cytotechnology, 3, 133 (1990)], the cells were suspended in 10 ml of IMDM-dFBS(10)-HT(1) [IMDM medium (manufactured by Invitrogen) containing 10% dFBS (manufactured by Invitrogen) and HT supplement (manufactured by Invitrogen) at 1× concentration) and the suspension was dispensed into a 96-well culture plate (manufactured by Iwaki Glass) at 100 μl/well. The cells were cultured in a 5% CO₂ incubator at 37° C. for 24 hours, and then its medium was changed to IMDM-dFBS(10) (IMM medium containing 10% dialyzed FBS), followed by culturing for 1 to 2 weeks, Since colonies of transformants showing IT-independent growth were observed, the transformants in the wells in which growth was observed were subjected to a DHFR gene amplification, and the amount of the antibody production was increased. Specifically, the cells were suspended in IMDM-dFBS(10) medium containing 50 nM MTX at a density of 1 to 2×10⁵ cells/ml, and the suspension was dispensed to a 24-well plate (manufactured by Iwaki Glass) at 0.5 ml/well. The cells were cultured in a 5% CO₂ incubator at 37° C. for 1 to 2 weeks to induce transformants showing 50 r MTX resistance. Regarding the transformants in wells in which growth was observed, the MTX concentration of the medium was increased to 200 nM, and then a transformant capable of growing in the IMDM-dFBS(10) medium containing 200 nM MTX and of producing the anti-CD20 human chimeric antibody in a large amount was finally obtained in the same manner as described above.

3. Culturing of an Antibody-Expressing Cell Line and Purification of an Antibody

The LCA Lectin-Resistant CHO/DG44 transformant cells capable of producing the anti-CD20 human chimeric antibody in a large amount obtained in the above item 2 was named R92-3-1. R92-3-1 has been deposited on Mar. 26, 2002, as FERM BP-7976 in International Patent Organism Depositary, National Insitute of Advanced Industrial Science and Technology (AIST Tsukuba Central 6, 1-1, Higashi 1-Chome Tsukaba-shi, Ibaraki-ken, Japan).

R92-3-1 was cultured in IMDM-dFBS(10) containing 200 nM M until the cells became confluent and was washed with Dulbecco's PBS (manufactured by Invitrogen), and then the medium was changed to EX-CELL301 (manufactured by JRH). The cells were cultured in a 5% CO₂ incubator at 37° C. for 7 days and the culture supernatant was collected. An anti-CD20 chimeric antibody was purified by using Prosep-A column (manufactured by Millipore) from the culture supernatant, and was named R92-3-1 antibody.

### EXAMPLE 7

Purification of an Anti-CD20 Chimeric Antibody Produced by Lectin-Resistant CHO/DG44 Cell and Evaluation of its Activity

1. Evaluation of Binding Activity of the Antibody Derived from Lectin-Resistant CHO/DG44 Cell (Immunofluorescent Method)

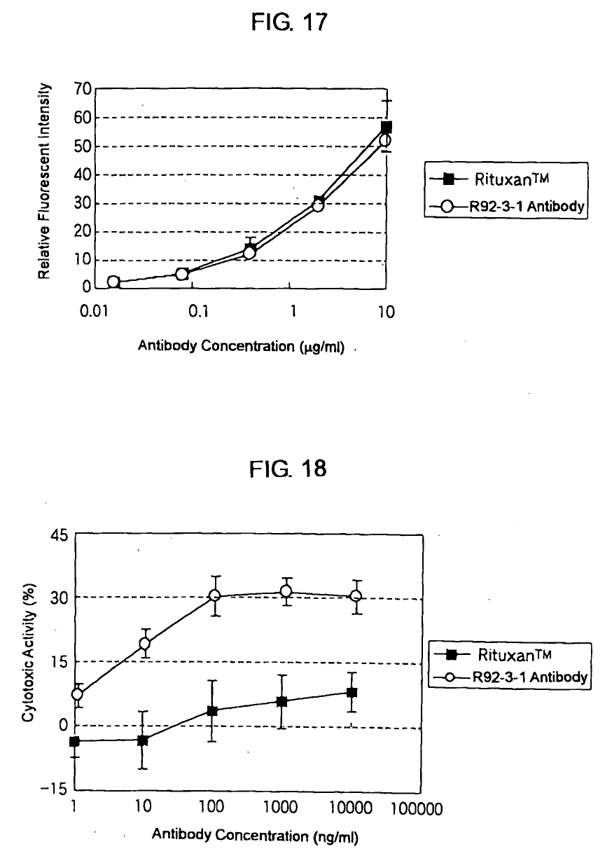

Binding activity of R92-3-1 antibody obtained in above item 3 of Example 6 to Raji cell line, in which CD20 is expressed, was examined according to the immunofluorescent method described in the above item 1 of Example 2 and compared with that of commercially available antibody Rituxan™ derived from ordinary CHO cell. As shown in FIG. 17, the fluorescent intensity was increased in dependence on antibody concentration in both R92-3-1 antibody and Rituxan™, and it was confirmed that they have almost similar binding activity.

2. Evaluation of In Vitro Cytotoxic Activity of the Antibody Derived from Lectin-Resistant CHO/DG44 Cell (ADCC Activity)

In order to evaluate in vitro ADCC activity of R92-3-1 antibody obtained in item 3 of Example 6, the ADCC activity was measured according to the method described in the above item 2 of Example 2. The ratio of the effector cell and the target cell, Raji cell, was 25:1, the final antibody concentration was 0.001 to 10 μg/mL, and the reaction was carried out at a total volume of 200 μl. The results are shown in FIG. 18.

The results show that R92-3-1 antibody derived from LCA lectin-resistant CHO/DG44 Cell has Higher ADCC Activity than Rituxan™.

3. Sugar Chain Analysis of the Antibody Derived from Lectin-Resistant CHO/DG44 Cell

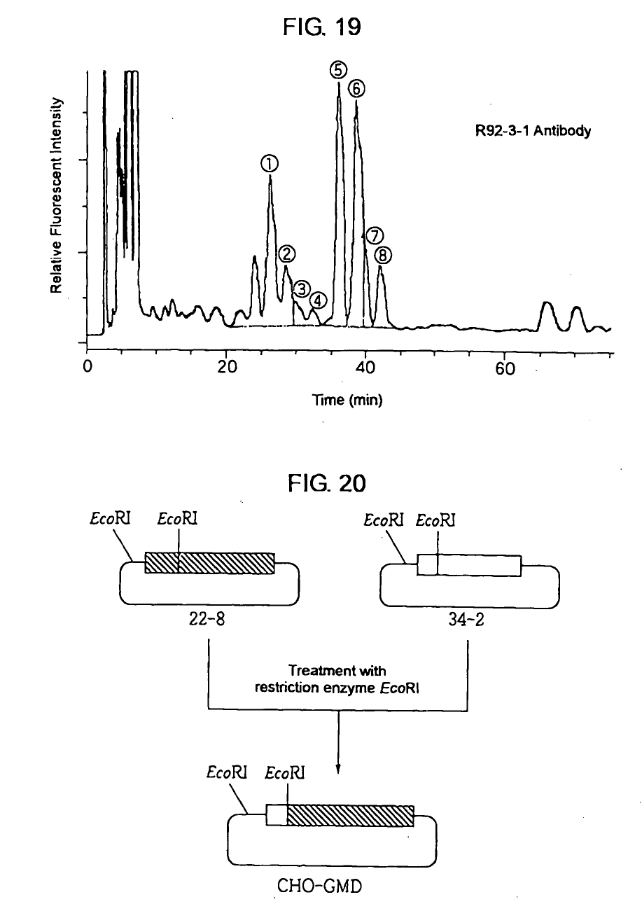

Sugar chain analysis of R92-3-1 antibody obtained in the above item 3 of Example 6 was carried out according to the method described in Example 3. The results are shown in FIG. 19. The sugar chain structures of peaks \mathring{1} to \mathring{8} in FIG. 19 are the same as those of peaks \mathring{1} to (a in FIG. 7, respectively.

In FIG. 19, the ratio of the α1,6-fucose-free sugar chain group was calculated from the area occupied by the peaks \mathring{1} to \mathring{4}, \mathring{9} and \mathring{10} among \mathring{1} to \mathring{10}. Also, the ratio of the α1,6-fucose-bound sugar chain group was calculated from the area occupied by the peaks \mathring{5} to \mathring{8} among \mathring{1} to \mathring{10}.

As a result, in R92-3-1 antibody, the ratio of the α1,6-fucose-not-bound sugar chain group was 33%, whereas the ratio of the c1,6-fucose-bound sugar chains was 67%. When compared with the sugar chain analysis of Rituxan™ carried out in Example 3, the antibody produced by LCA lectin-resistant CHO/DC44 cells has a higher ratio of α1,6-fucose-not bound sugar chains.

### EXAMPLE 8

Preparation of CHO Cell-Derived GMD Gene:

1. Determination of cDNA Sequence of CHO Cell-Derived GMD gene

(1) Preparation of cDNA of CHO Cell-Derived GND Gene (Preparation of Partial cDNA Excluding 5′- and 3′-terminal Sequences)

cDNA of rodents-derived GMD was searched in a public data base (BLAST) by using cDNA sequence of a human-derived GND (GenBank Accession No. AF042377) registered at Genank as a query, and three kinds of mouse EST sequences were obtained (GenBank Accession Nos. BE986856, BF158988 and BE284785). By ligating these EST sequences, a deduced cDNA sequence of mouse GMD was determined.

On the base of cDNA sequence of the mouse-derived GMD, a 28 mer primer having the sequence represented by SEQ D NO:32, a 27 mer primer having the sequence represented by SEQ ID NO:33, a 25 mer primer having the sequence represented by SEQ ID NO:34, a 24 mer primer having the sequence represented by SEQ ID NO:35 and a 25 mer primer having the sequence represented by SEQ D NO:36 were prepared.

Next, CHO/DG44 cell was subcultured in a 5% CO₂ incubator at 37° C., followed by culturing. After culturing, a total RNA was prepared from 1×10⁷ cells of each cell line by using RNeasy Protect Mini Kit (manufactured by QIAGEN) according to the manufacture's instructions, and a single-stranded cDNA was synthesized from 5 μg of each RNA in a 20 μl of a reaction mixture using RT-PCR (manufactured by GIBCO BRL) according to the manufacture's instructions.

Next, in order to amplify the CHO cell-derived cDNA, PCR was carried out by the following method. Specifically, 20 μl of a reaction mixture [1×Ex Taq buffer (manufactured by Takara Shuzo), 0.2 nmM dNTPs, 0.5 unit of Ex Taq polymerase (manufactured by Takara Shuzo) and 0.5 μM of two synthetic DNA primers] containing 0.5 μl of the CHO cell-derived single-stranded cDNA as the template was prepared. In this case, combinations of SEQ ID NO:32 with SEQ ID NO:33, SEQ ID NO:34 with SEQ ID NO:33, SEQ ID NO:32 with SEQ ID NO:35 and SEQ ID NO:32 with SEQ ID NO:36 were used as the synthetic DNA primers. The reaction was carried out by using DNA Thermal Cycler 480 (manufactured by Perkin Elmer) by heating at 94° C. for 5 minutes and subsequent 30 cycles of heating at 94° C. for 1 minute and 68° C. for 2 minutes as one cycle.

The PCR reaction mixture was subjected to agarose electrophoresis for fractionation to find that a DNA fragment of about 1.2 kbp was amplified in the PCR product when synthetic DNA primers of SEQ ID NOs:32 and 33 were used, a fragment of about 1.1 kbp was amplified in the PCR product when synthetic DNA primers of SEQ ID NOs:33 and 34 were used, a fragment of about 350 bp was amplified in the PCR product when synthetic DNA primers of SEQ ID NOs:32 and 35 were used and a fragment of about 1 klbp was amplified in the PCR product when synthetic DNA primers of SEQ ID NOs:32 and 36 were used. The DNA fragments were recovered by using Gene Clean II Kit (manufactured by BIO101) in accordance with the manufacture's instructions. The recovered DNA fragments were ligated to a pT7Blue(R) vector (manufactured by Novagen) using DNA Ligation Kit (manufactured by Takara Shuzo), and E. coli DH (manufactured by Toyobo) was transformed by using the obtained recombinant plasmid DNA samples to thereby obtain plasmids 22-8 (having a DNA fragment of about 1.2 kbp amplified from synthetic DNA primers of SEQ ID NO:32 and SEQ ID NO:33), 23-3 (having a DNA fragment of about 1.1 kbp amplified from synthetic DNA primers of SEQ ID NO:34 and SEQ ID NO:33), 31-5 (a DNA fragment of about 350 bp amplified from synthetic DNA primers of SEQ ID NO:32 and SEQ ID NO:35) and 34-2 (having a DNA fragment of about 1 kbp amplified from synthetic DNA primers of SEQ]ID NO:32 and SEQ ID NO:36). The cDNA sequence of CHO cell-derived GMD contained in these plasmids was determined by using a DNA sequencer ABI PRISM 377 (manufactured by Perkin Elmer) (since a sequence of 28 bases in downstream of the initiation codon methionine in the 5′-terminal side and a sequence of 27 bases in upstream of the termination codon in the 3′-terminal side are originated from synthetic oligo DNA sequences, they are mouse GMD cDNA sequences) in the usual method.

In addition, the following steps were carried out in order to prepare a plasmid in which cDNA fragments of the CHO cell-derived GMD contained in the plasmids 22-8 and 34-2 are combined. After 1 μg of the plasmid 22-8 was allowed to react with a restriction enzyme EcoRI (manufactured by Takara Shuzo) at 37° C. for 16 hours, the digest was subjected to agarose electrophoresis, and then a DNA fragment of about 4 kbp was recovered by using Gene Clean III Kit (manufactured by BIO101) in accordance with the manufacture's instructions. After 2 μg of the plasmid 34-2 was allowed to react with a restriction enzyme EcoRI at 37° C. for 16 hours, the digest was subjected to agarose electrophoresis and then a DNA fragment of about 150 bp was recovered by using Gene Clean II Kit (manufactured by BIO101) in accordance with the manufacture's instructions. The recovered DNA fragments were respectively subjected to terminal dephosphorylation by using Calf Intestine Alkaline Phosphatase (manufactured by Takara Shuzo) and then ligated by using DNA Ligation Kit (manufactured by Takara Shuzo), and E. coli DHSA (manufactured by Toyobo) was transformed by using the obtained recombinant plasmid DNA to obtain a plasmid CHO-GMD (FIG. 20).

(2) Determination of the 5′-terminal Sequence of CHO Cell-Derived GMD cDNA

A 24 mer primer having the nucleotide sequence represented by SEQ ID NO:37 was prepared from 5′-terminal side non-coding region nucleotide sequences of CHO cell-derived GMD cDNA, and a 32 mer primer having the nucleotide sequence represented by SEQ ID NO:38 from CHO cell-derived GMD cDNA sequence was prepared, and PCR was carried out by the following method to amplify cDNA. Then, 20 μl of a reaction mixture [1×Ex Taq buffer (manufactured by Takara Shuzo), 0.2 mM dNTPs, 0.5 unit of Ex Taq polymerase (manufactured by Takara Shuzo) and 0.5 μM of the synthetic DNA primers of SEQ ID NO:37 and SEQ ID NO:38] containing 0.5 μl of the single-stranded cDNA as the template derived from CHO cell was prepared, and the reaction was carried out therein by using DNA Thermal Cycler 480 (manufactured by Perkin Elmer) by heating at 94° C. for 5 minutes, subsequent 20 cycles of heating at 94° C. for 1 minute, 55° C. for 1 minute and 72° C. for 2 minutes as one cycle and further 18 cycles of heating at 94° C. for 1 minute and 68° C. for 2 minutes as one cycle. After fractionation of the PCR reaction mixture by agarose electrophoresis, a DNA fragment of about 300 bp was recovered by using Gene Clean II Kit (manufactured by BIO101) in accordance with the manufacture's instructions. The recovered DNA fragment was ligated to a pT7Blue(R) vector (manufactured by Novagen) using DNA Ligation Kit (manufactured by Takara Shuzo), and E. coli DH5α (manufactured by Toyobo) was transformed by using the obtained recombinant plasmid DNA samples to thereby obtain a plasmid 5′GMD. By using DNA Sequencer 377 (manufactured by Applied Biosystems), the nucleotide sequence of 28 bases in downstream of the initiation methionine of CHO cell-derived GMD cDNA contained in the plasmid was determined.

(3) Determination of the 3′-terminal Sequence of CHO Cell-Derived GMD cDNA

In order to obtain the 3′-terminal cDNA sequence of a CHO cell-derived GMD, RACE method was carried out by the following method. A single-stranded cDNA for 3′ RACE was prepared from the CHO cell-derived RNA by using SMART™ RACE cDNA Amplification Kit (manufactured by CLONTECH) in accordance with the manufacture's instructions. In this case, PowerScript™ Reverse Transcriptase (manufactured by CLONTECH) was used as the reverse transcriptase. The single-stranded cDNA after the preparation was diluted 10 folds with the Tricin-EDTA buffer attached to the kit and used as the template of PCR.

Next, 20 μl of a reaction mixture [ExTaq buffer (manufactured by Takara Shuzo), 0.2 mM dNTPs, 0.5 unit of EX Taq polymerase (manufactured by Takara Shuzo), 0.5 μM of the 24 mer synthetic DNA primer shown in SEQ ID NO:39 prepared on the base of cDNA sequence of the CHO cell-derived GM) determined in item (1)] and 1× concentration of Universal Primer Mix (attached to SMART™ RACE cDNA Amplification Kit; manufactured by CLONTECH] containing 1 μl of the cDNA for 3′ RACE as the template was prepared, and PCR was carried out by using DNA Thermal Cycler 480 (manufactured by Perkin Elmer) by heating at 94° C. for 5 minutes and subsequent 30 cycles of heating at 94° C. for 1 minute and 68° C. for 2 minutes as one cycle.

After completion of the reaction, 1 μl of the PCR reaction mixture was diluted 20 folds with Tricin-EDTA buffer (manufactured by CLONTECH). Then, 20 μl of a reaction mixture [ExTaq buffer (manufactured by Takara Shuzo), 0.2 mM dNTPs, 0.5 unit of EX Taq polymerase (manufactured by Takara Shuzo), 0.5 μM of the 25 mer synthetic DNA primer shown in SEQ ID NO:40 [prepared on the base of the cDNA sequence of CHO cell-derived GMD determined in item (1)] and 0.5 μM of Nested Universal Primer (attached to SMART™ RACE cDNA Amplification Kit; manufactured by CLONTECH) containing 1 μl of the 20 folds-diluted aqueous solution as the template] was prepared, and the reaction was carried out by using DNA Thermal Cycler 480 (manufactured by Perkin Elmer) by heating at 94° C. for 5 minutes and subsequent 30 cycles at 94° C. for 1 minute and 68° C. for 2 minutes as one cycle.

After completion of the reaction, the PCR reaction mixture was subjected to agarose electrophoresis for fractionation and then a DNA fragment of about 700 bp was recovered by using Gene Clean II Kit (manufactured by BIO101) in accordance with the manufacture's instructions. The recovered DNA fragment was ligated to a pT7Blue(R) vector (manufactured by Novagen) by using DNA Ligation Kit (manufactured by Takara Shuzo), and E. coli DH5α (manufactured by Toyobo) was transformed by using the obtained recombinant plasmid DNA to thereby obtain a plasmid 3′GM. By using DNA Sequencer 377 (manufactured by Applied Biosystems), the nucleotide sequences of 27 bases in upstream of the termination codon and 415 bases in the non-coding region in the 3′-terminal of CHO cell-derived GMD cDNA contained in the plasmid were determined.

The full length cDNA sequence of the CHO-derived GMD gene determined in items (1), (2) and (3) and the corresponding amino acid sequence are shown in SEQ ID NOs:41 and 61, respectively.

2. Determination of a Genomic Sequence Containing CHO/DG44-derived Cell GMD gene

A 25 mer primer having the nucleotide sequence represented by SEQ ID NO; 56 was prepared from the cDNA sequence of mouse Go determined in item 1 of Example 8. Next, a CHO cell-derived genomic DNA was obtained by the following method. CHO/DG44 cell was suspended in IMDM-dFBS(10)—HT(1) medium [IMDM-dFBS(10) medium comprising 1× concentration of HT supplement (manufactured by Invitrogen)] at a density of 3×10⁵ cells/ml, and the suspension was dispensed into a 6 well flat bottom tissue culture plate for adhesion cell (manufactured by Greiner) at 2 ml/well. After culturing them at 37° C. in a 5% CO₂ incubator until the cells became confluent on the plate, genomic DNA was prepared from the cells on the plate by a known method [Nucleic Acids Research, 3, 2303 (1976)] and dissolved overnight in 150 μl of TE-RNase buffer (pH 8.0) (10 mmol/l Tris-HCl, 1 mmol/l EDTA, 200 μg/ml RNase A).

Next, 100 ng of the obtained CHO/DG44 cell-derived genomic DNA and 20 μl of a reaction mixture [1×Ex Taq buffer (manufactured by Takara Shuzo), 0.2 mM dNTPs, 0.5 unit of EX Taq polymerase (manufactured by Takara Shuzo) and 0.5 μM synthetic DNA primers of SEQ ID NO:35 and SEQ ID NO:56] were prepared, and PCR was carried out by using DNA Thermal Cycler 480 (manufactured by Perkin Elmer) by heating at 94° C. for 5 minutes and subsequent 30 cycles of heating at 94° C. for 1 minute and 68° C. for 2 minutes as one cycle. After completion of the reaction, the PCR reaction mixture was subjected to agarose electrophoresis for fractionation and then a DNA fragment of about 100 bp was recovered by using Gene Clean II Kit (manufactured by BIO101) in accordance with the manufacture's instructions. The recovered DNA fragment was ligated to a pT7Blue(R) vector (manufactured by Novagen) by using DNA Ligation Kit (manufactured by Takara Shuzo), and E. coli DHS5α (manufactured by Toyobo) was transformed by using the obtained recombinant plasmid DNA, thereby obtaining a plasmid ex3. By using DNA Sequencer 377 (manufactured by Applied Biosystems), the nucleotide sequence of CHO cell-derived genomic DNA contained in the plasmid was determined. The determined nucleotide sequence is shown in SEQ ID NO: 57.

Next, a 25 mer primer having the nucleotide sequence represented by SEQ ID NO:58 and a 25 mer primer having the nucleotide sequence represented by SEQ ID NO:59 were prepared on the base of the cDNA sequence of CHO cell-derived GMD determined in item 1 of Example 8. Next, 100 ng of the CHO/DG44-derived genomic DNA and 20 μl of a reaction mixture [1×Ex Taq buffer (manufactured by Takara Shuzo), 0.2 mM dNTPs, 0.5 unit of EX Taq polymerase (manufactured by Takara Shuzo) and 0.5 μM synthetic DNA primers of SEQ ID NO:58 and SEQ ID NO:59] were prepared, and PCR was carried out by using DNA Thermal Cycler 480 (manufactured by Perkin Elmer) by heating at 94° C. for 5 minutes and subsequent 30 cycles of heating at 94° C. for 1 minute and 68° C. for 2 minutes as one cycle.

After completion of the reaction, the PCR reaction mixture was subjected to agarose electrophoresis for fractionation and then a DNA fragment of about 200 bp was recovered by using Gene Clean II Kit (manufactured by BIO111) in accordance with the manufacture's instructions. The recovered DNA fragment was ligated to a pT7Blue(R) vector (manufactured by Novagen) by using DNA Ligation Kit (manufactured by Takara Shuzo), and E. coli DH5α (manufactured by Toyobo) was transformed by using the obtained recombinant plasmid DNA, thereby obtaining a plasmid ex4. By using DNA Sequencer 377 (manufactured by Applied Biosystems), the nucleotide sequence of CHO cell-derived genomic DNA contained in the obtained plasmid was determined. The determined nucleotide sequence is shown in SEQ ID NO:60.

### EXAMPLE 9

Preparation of Various CHO Cell-Derived Genes Encoding Enzymes Relating to the Sugar Chain Synthesis:

1. Determination of CHO Cell-derived FX cDNA Sequence

(1) Extraction of Total RNA Derived from CHO/DG44 Cell

CHO/DG44 cells were suspended in IMDM medium containing 10% fetal bovine serum (manufactured by Life Technologies) and 1× concentration HT supplement (manufactured by Life Technologies), and 15 ml of the suspension was inoculated into a T75 tissue culture flask for adhesion cell culture (manufactured by Greiner) at a density of 2×10⁵ cells/ml. On the second day after culturing at 37° C. in a 5% CO₂ incubator, 1×10⁷ cells were recovered and a total RNA was extracted therefrom by using RNAeasy (manufactured by QIAGEN) in accordance with the manufacture's instructions.

(2) Preparation of CH0-DG44 Cell-Derived Single-Stranded cDNA

The total RNA prepared in item (1) was dissolved in 45 μl of sterile water, and 1 μl of RQ1 RNase-Free DNase (manufactured by Promega), δ 0 of the attached 10× DNase buffer and 0.5 μl of RNasin Ribonuclease Inhibitor (manufactured by Promega) were added thereto, followed by reaction at 37° C. for 30 minutes to degrade genomic DNA contaminated in the sample. After the reaction, the total RNA was purified again by using RNAeasy (manufactured by QIAGllN and dissolved in 50 μl of sterile water.

In a 20 μl of reaction mixture using oligo(dT) as a primer, single-stranded cDNA was synthesized from 3 μg of the obtained total RNA samples by carrying out reverse transcription reaction using SUPERSCRIPT™ Preamplification System for First Strand cDNA Synthesis (manufactured by Life Technologies) in accordance with the manufacture's instructions. A 50 folds-diluted aqueous solution of the reaction mixture was used in the cloning of GFPP and FX. This was stored at —SO° C. until use.

(3) Preparation of a cDNA Partial Fragment of Chinese Hamster-Derived FX

A cDNA partial fragment derived from Chinese hamster-derived FX was prepared by the following procedure. First, primers (shown in SEQ ID NOs:42 and 43) specific for nucleotide sequences common to a human FX cDNA (Genebank Accession No. U58766) and a mouse Fx EDNA (Genebank Accession No. M30127), were designed.

Next, 25 μl of a reaction mixture [ExTaq buffer (manufactured by Takara Shuzo), 0.2 mM dNTPs and 0.5 μmol/l gene-specific primers (SEQ ID NOs:42 and 43)] containing 1 μl of the CHO/DG44-derived single-stranded cDNA prepared in item (2) was prepared, and polymerase chain reaction (CR) was carried out by using a DNA polymerase ExTaq (manufactured by Takara Shuzo). The PCR was carried out by heating at 94° C. for 5 minutes, subsequent 30 cycles of heating at 94° C. for 1 minute, 58° C. for 2 minutes and 72° C. for 3 minutes as one cycle, and final heating at 72° C. for 10 minutes.

After the PCR, the reaction mixture was subjected to 2% agarose gel electrophoresis, and a specific amplified fragment of 301 bp was purified by using QiaexII Gel Extraction Kit (manufactured by QIAGEN) and eluted with 20 μl of sterile water (hereinafter, the method was used for the purification of DNA fragments from agarose gel). Into a plasmid pCR2.1, 4 μl of the amplified fragment was inserted by TOPO TA Cloning Kit (manufactured by Invitrogen) in accordance with the manufacture's instructions attached thereto, and E coli DH5α was transformed with the reaction mixture by the method of Cohen et al. [Proc. Natl. Acad. Sci. USA, 69, 2110 (1972)] (hereinafter, the method was used for the transformation of E. coli). Plasmid DNA was isolated in accordance with a known method [Nucleic Acids Research, 7, 1513 (1979)] (hereinafter, the method was used for the isolation of plasmid) from the obtained several kanamycin-resistant colonies to obtain 2 clones into which cDNA partial fragments of Fx were respectively inserted. They are referred to as pCRFX clone 8 and pCRFX clone 12.

The nucleotide sequence of the cDNA inserted into each of the FX clone 8 and FX clone 12 was determined by using DNA Sequencer 377 (manufactured by Applied Biosystems) and Bigye Terminator Cycle Sequencing FS Ready. Reaction kit (manufactured by Applied Biosystems) in accordance with the method of the manufacture's instructions. It was confirmed that each of the inserted cDNA whose sequence was determined encodes open reading fame (ORF) partial sequence of the Chinese hamster-derived FX.

(4) Synthesis of a Single-Stranded cDNA for RACE

Single-stranded cDNA samples for 5′ and 3′ RACE were prepared from the CHO/DG44 total RNA extracted in item (1) by using SMART™ RACE cDNA Amplification Kit (manufactured by CLONTECH) in accordance with the manufacture's instructions. As the reverse transcriptase, PowerScript™ Reverse Transcriptase (manufactured by CLONTECH) was used. Each of the prepared single-stranded cDNA was diluted 10 folds with the Tricin-EDTA buffer attached to the kit and used as the template of PCR Based on the partial sequence of Chinese hamster-derived Fx determined in item (3), primers FXGSP1-1 (SEQ ID NO:44) and FXGSP1-2 (SEQ ID) NO:45) for the Chinese hamster FX-specific 5′ RACE and primers FXGSP2-1 (SEQ ID NO:46) and FXGSP2-2 (SEQ ID NO:47) for the Chinese hamster FX-specific 3′ RACE were designed.

Next, polymerase chain reaction (PCR) was carried out by using Advantage2 PCR Kit (manufactured by CLONTECH), by preparing 50 μl of a reaction mixture [Advantage2 PCR buffer (manufactured by CLONTECH), 0.2 mM dNTPs, 0.2 μmol/l Chinese hamster FX-specific primers for RACE and 1× concentration of common primers (manufactured by CLONTECH)] containing 1 μl of the CHO/DG44-derived single-stranded cDNA for RACE prepared in item (4).

The PCR was carried out by repeating 20 cycles of heating at 94° C. for 5 seconds, 68° C. for 10 seconds and 72° C. for 2 minutes as one cycle.

After completion of the reaction, 1 μl of the reaction mixture was diluted 50-folds with the Tricin-EDTA buffer, and 1 μl of the diluted solution was used as a template, the reaction mixture was again prepared, and the PCR was carried out under the same conditions. The combination of primers used in the first and second PCRs and the length of amplified DNA fragments by the PCRs are shown in Table 2.

TABLE 2
Combination of primers used in Chinese hamster-derived
FX cDNA RACE PCR and the size of PCR products

| | | |
|---|---|---|
| FX-specific | | PCR-amplified |
| primers | Common primers | product size |

5′ RACE

| | | | |
|---|---|---|---|
| First | FXGSP1-1 | UPM (Universal primer mix) | |
| Second | FXGSP1-2 | NUP (Nested Universal | 300 bp |
| | | primer) | |
| 3′ RACE | | | |
| First | FXGSP2-1 | UPM (Universal primer mix) | |
| Second | FXGSP2-2 | NUP (Nested Universal | 1,100 bp |
| | | primer) | |

After the PCR, the reaction mixture was subjected to 1% agarose gel electrophoresis, and the specific amplified fragment of interest was purified by using QiaexII Gel Extraction Kit (manufactured by QIAGEN) and eluted with 20 μl of sterile water. Into a plasmid pCR2.1, 4 μl of the amplified fragment was inserted, and E. coli DH5α was transformed by using the reaction mixture in accordance with the manufacture's instructions attached to TOPO TA Cloning Kit (manufactured by Invituogen).

Plasmid DNAs were isolated from the appeared several kanamycin-resistant colonies, and 6 cDNA clones containing Chinese hamster FX 5′ region were obtained. They are referred to as FX5′ clone 25, FX5′ clone 26, FX5′ clone 27, FX5′ clone 28, FX5′ clone 31 and FX5′ clone 32.

In the same manner, S cDNA clones containing Chinese hamster FX 3′ region were obtained. These FX3′ clones are referred to as FX3′ clone 1, FX3′ clone 3, FX3′ clone 6, FX3′ clone 8 and MX3′ clone 9.

The nucleotide sequence constituting the cDNA of each of the clones obtained by the 5′ and 3′ RACE was determined by using DNA Sequencer 377 (manufactured by Applied Biosystems) in accordance with the method described in the manufacture's instructions. By comparing the cDNA nucleotide sequences determined by the method, reading errors of nucleotide in PCR were excluded, and the full length nucleotide sequence of Chinese hamster-derived FX cDNA was determined. The determined sequence is represented by SEQ ID NO:48. ORF of SEQ ID NO:48 corresponds to nucleotides at positions 95 to 1060, and the amino acid sequence corresponding to nucleotides at positions 95 to 1057 excluding the termination codon is represented by SEQ ID NO:62.

2. Determination of a GFPP cDNA Sequence of CHO Cell

(1) Preparation of a cDNA Partial Fragment of Chinese Hamster-Derived GFPP

A cDNA partial fragment of Chinese hamster GFPP was prepared by the following procedure.

First, a nucleotide sequence of a human-derived GFPP cDNA (Genebank Accession No. AF017445), mouse EST sequences having high homology with the nucleotide sequence (Genebank Accession Nos. AI467195, AA422658, BE304325 and AI466474) and rat EST sequences (Genebank Accession Nos. BF546372, AI058400 and AW144783), registered at public data bases, were compared, and primers of GFPP FW9 and GFPP RV9 (SEQ ID NOs:49 and 50), specific for rat GFPP were designed on a highly preserved region among these three species.

Next, polymerase chain reaction (PCR) was carried out by using a DNA polymerase ExTaq (manufactured by Takara Shuzo), by preparing 25 μl of a reaction mixture [ExTaq buffer (manufactured by Takara Shuzo), 0.2 mM dNTPs and 0.5 μmol/l GFPP-specific primers GFPP FW9 and (GFPP RV9 (SEQ ID NOs:49 and 50)] containing 1 μl of the CHO/DG44-derived single-stranded cDNA prepared in item 1(2). The PCR was carried out by heating at 94° C. for 5 minutes, subsequent 30 cycles of heating at 94° C. for 1 minute, 58° C. for 2 minutes and 72° C. for 3 minutes as one cycle, and final heating at 72° C. for 10 minutes.

After the PCR, the reaction mixture was subjected to 2% agarose gel electrophoresis, and a specific amplified fragment of 1.4 Kbp was purified using QuiaexII Gel Extraction Kit (manufactured by QIAGEN) and eluted with 20 μl of sterile water. Into a plasmid pCR2.1, 4 μl of the amplified fragment was inserted to insert in accordance with the manufacture's instructions attached to TOPO TA Cloning Kit (manufactured by Invitrogen), and E. coli DH5α was transformed by using the reaction mixture.

Plasmid DNAs were isolated from the appeared several kanamycin-resistant colonies, and 3 clones transfected with GFPP cDNA partial fragments were obtained. They are referred to as GFPP clone 8, GFPP clone 11 and GFPP clone 12.

The nucleotide sequence of the cDNA inserted into each of the GFPP clone 8, GFPP clone 11 and GFPP clone 12 was determined by using DNA Sequencer 377 (manufactured by Applied Biosystems) and BigDye Terminator Cycle Sequencing FS Ready Reaction kit (manufactured by Applied Biosystems) in accordance with the method described in the manufacture's instructions It was confirmed that the inserted cDNA whose sequence was determined encodes a partial sequence of the open reading frame (ORE) of the Chinese hamster-derived GFPP.

(2) Determination of Full Length cDNA of Chinese Hamster-Derived GFPP by RACE Method

Based on the Chinese hamster FX partial sequence determined in item 2(1), primers GFPP GSP1-1 (SEQ ID NO:52) and GFPP GSP1-2 (SEQ ID NO:53) for the Chinese hamster FX-specific 5′ RACE and primers GFPP GSP2-1 (SEQ ID NO:54) and GFPP GSP2-2 (SEQ ID NO:55) for the Chinese hamster GFPP-specific 3′ RACE were designed.

Next, polymerase chain reaction (PCR) was carried out by using Advantage2 PCR Kit (manufactured by CLONTECH), by preparing 50 μl of a reaction mixture [Advantage2 PCR buffer (manufactured by CLONTECH), 0.2 mM dNTPs, 0.2 μmol/l Chinese hamster GFPP-specific primers for RACE and Ix concentration of common primers (manufactured by CLONTECH)] containing 1 μl of the CHO/DG44-derived single-stranded cDNA for RACE prepared in item (4).

The PCR was carried out by repeating 20 cycles of heating at 94° C. for 5 seconds, 68° C. for 10 seconds and 72° C. for 2 minutes as one cycle.

After completion of the reaction, 1 μl of the reaction mixture was diluted 50 folds with the Tricin-EDTA buffer. By using 1 μl of the diluted solution as a template, the reaction mixture was again prepared and the PCR was carried out under the same conditions. The combination of primers used in the first and second PCRs and the size of amplified DNA fragments by the PCRs are shown in Table 3.

TABLE 3
Combination of primers used in Chinese hamster-derived
GFPP cDNA RACE PCR and the size of PCR products

| | | |
|---|---|---|
| | | PCR- |
| GFPP-specific | | amplified |
| primers | Common primers | product size |

5′ RACE

| | | | |
|---|---|---|---|
| First | GFPPGSP1-1 | UPM (Universal primer mix) | |
| Second | GFPPGSP1-2 | NUP (Nested Universal | 1,100 bp |
| | | primer) | |
| 3′ RACE | | | |
| First | GFPPGSP2-1 | UPM (Universal primer mix) | |
| Second | GFPPGSP2-2 | NUP (Nested Universal | 1,400 bp |
| | | primer) | |

After the PCR, the reaction mixture was subjected to 1% agarose gel electrophoresis, and the specific amplified fragment of interest was purified using QiaexII Gel Extraction Kit (manufactured by QIAGEN) and eluted with 20 μl of sterile water. Into a plasmid pCR2.1, 4 μl of the amplified fragment was inserted and E. coli DH5α was transformed with the reaction mixture in accordance with the manufacture's instructions attached to TOPO TA Cloning Kit (manufactured by Invitrogen).

Plasmid DNAs were isolated from the appeared several kanamycin-resistant colonies to obtain 4 cDNA clones containing Chinese hamster GFPP 5′ region. They are referred to as GFPP5′ clone 1, GFPP5′ clone 2, GFPP5′ clone 3 and GFPP5′ clone 4.

In the same manner, 3 cDNA clones containing Chinese hamster GFPP 3′ region were obtained. They are referred to as GFPP3′ clone 10, GFPP3′ clone 16 and GFPP3′ clone 20.

The nucleotide sequence of the cDNA of each of the clones obtained by the 5′ and 3′ RACE was determined by using DNA Sequencer 377 (manufactured by Applied Biosystems) in accordance with the method described in the manufacture's instructions. By comparing the cDNA nucleotide sequences after the nucleotide sequence determination, reading errors of bases in PCR were excluded and the full length nucleotide sequence of Chinese hamster GFPP cDNA was determined. The determined sequence is shown in SEQ ID NO:51. ORF of SEQ D NO:51 corresponds to nucleotides at positions 27 to 1799, and the amino acid sequence corresponding to nucleotides at positions 27 to 1796 excluding the termination codon is represented by SEQ ID NO:63.

### EXAMPLE 10

Evaluation of Activity of Anti-CD20 Chimeric Antibodies Having a Different Ratio of Antibody Molecules to Which an α1,6-fucose-free Sugar Chain is Bound

1. Preparation of Anti-CD20 Chimeric Antibodies Having a Different Ratio of Antibody Molecules to Which an α1,6-fucose-free Sugar Chain is Bound

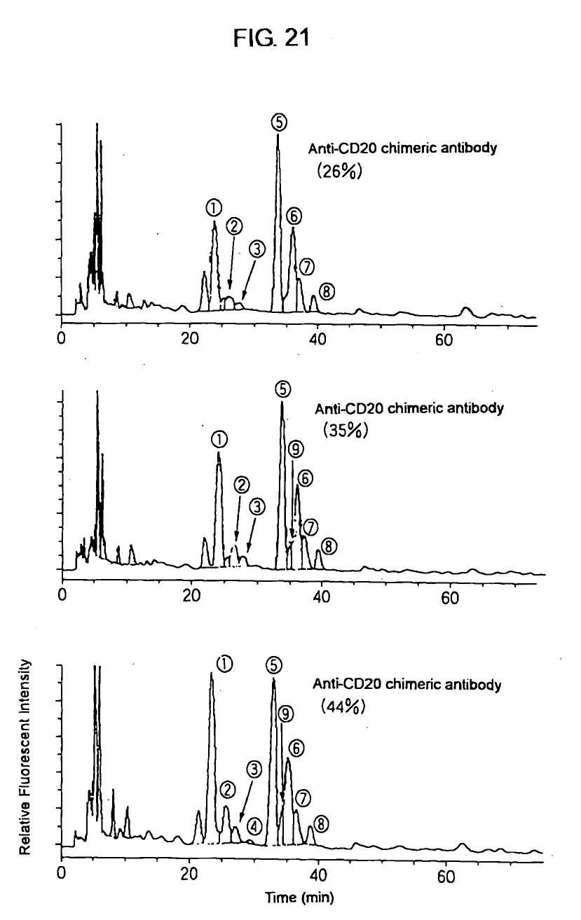

KM3065 purified in item 3 of Example 1 was mixed with CHO produced-Rituxan™ at a ratio of KM3065: Rituxan™=24:66, 34:56 or 44:46. Sugar chain analysis of these samples was carried out in accordance with the method of Example 3. Ratios of the antibody molecules to which an α1,6-fucose-free sugar chain was bound were 26%, 35% and 44%, respectively. Hereinafter, these samples are called anti-CD20 chimeric antibody (26%), anti-CD20 chimeric antibody (35%) and anti-CD20 chimeric antibody (44%). Results of the sugar chain analysis of each sample are shown in FIG. 21.

2. Evaluation of Binding Activity to CD20-Expressing Cell Line (Immunofluorescent Method)

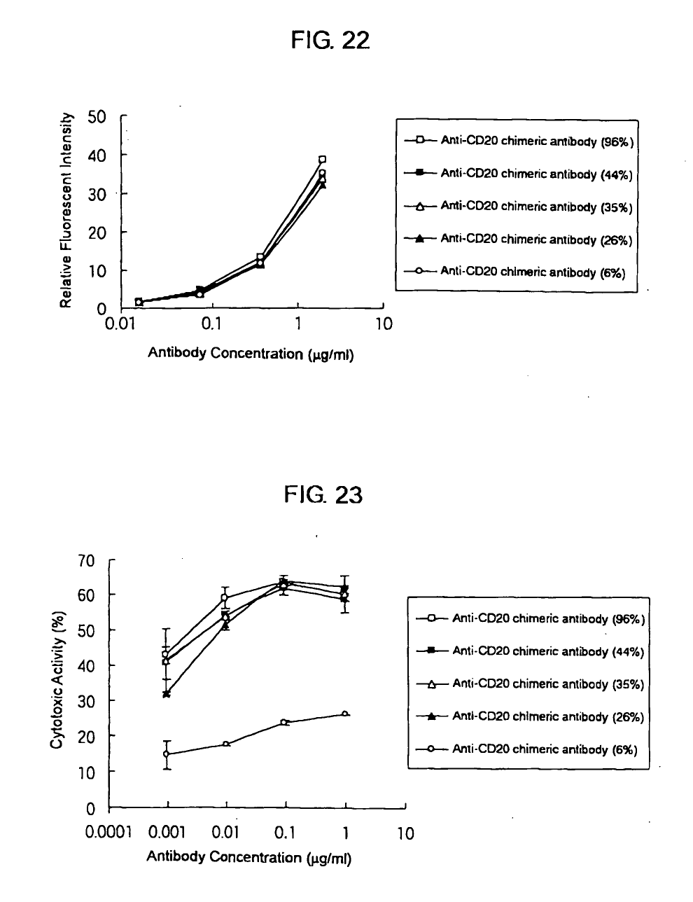

Binding activities of a total of five antibodies, including the 3 anti-CD20 chimeric antibodies having a different ratio of sugar chain of antibody molecules to which an α1,6-fucose-free sugar chain is bound, prepared in item 1 of Example 10, and KM3065 and Rituxan™ whose sugar chain analysis was carried out in Example 3 (referred to as “anti-CD20 chimeric antibody (96%)” and “anti-CD20 chimeric antibody (6%)”, respectively), were measured by the immunofluorescent method described in item 1 of Example 2. As shown in FIG. 22, all of these antibodies showed almost the same binding activity to the CD20-positive Raji cell (JCRB 9012) at an antibody concentration of 0.016 to 2 μg/ml, and it was found that the ratio of sugar chain of antibody molecules to which an α1,6-fucose-free sugar chain is bound does not have influence on the antigen-binding activity of antibodies.

3. Evaluation of Cytotoxic Activity to CD20-Expressing Cell Line (⁵¹Cr Release Method)

The ADCC activity against a CD20-positive human B lymphoid cell line WL2-S (ATCC CRY 8885) was measured as follows using effector cells collected from a healthy donor A.

(1) Preparation of Target Cell Suspension

After 2×10′ cells of the WIL2-S cell were prepared, the cells were isotope-labeled by adding 3.7 MBq equivalents of a radioactive substance Na₂⁵¹CrO₄ and carrying out the reaction at 37° C. for 1 hour. After the reaction, the cells were washed three times by repeating their suspension in PRM[1640-FCS(10) medium and subsequent centrifugation, re-suspended in the medium and then allowed to stand at 4° C. for 30 minutes in ice for spontaneous dissociation of the radioactive substance. After centrifugation, the cells were adjusted to a density of 2×10⁵ cells/ml by adding 10 ml of the medium and used as the target cell suspension.

(2) Preparation of Human Effector Cell Suspension

After 50 ml of peripheral blood was collected from a health person, 0.5 ml of heparin sodium (manufactured by Shimizu Pharmaceutical) was added thereto, followed by gently mixing. The mixture was centrifuged (800×g, 20 minutes) using Lymphoprep (manufactured by AXIS SHIELD) in accordance with the manufacture's instructions attached thereto to separate a mononuclear leukocyte layer. After washing with a medium three times by centrifugation (1,400 rpm, 5 minutes), the cells were resuspended by using the medium to a density of 2×10⁶ cells/ml and used as the human effector cell suspension.

(3) Measurement of ADCC Activity

The target cell suspension prepared in (1) (50 μl) was dispensed into wells of a 96-well U-bottom plate (manufactured by Falcon) (1 cells/well). Next, 100 μl of the human effector cell suspension prepared in (2) was dispensed (2×10⁵ cells/well, the ratio of human effector cells to target cells becomes 20:1). Subsequently, various anti-CD20 chimeric antibodies having a different ratio of (α1,6-fucose-free sugar chain group was bound were added thereto to give a respective final concentration of 0.001 to 1 μg/ml and then allowed to react at 37° C. for 4 hours. After the reaction, the plate was subjected to centrifugation and the amount of ⁵¹Cr in the supernatant was measured using a γ-counter. The amount of the spontaneously released ⁵¹Cr was calculated by carrying out the same procedure using the medium alone instead of the human effector cell suspension and antibody solution and measuring amount of ⁵¹Cr in the supernatant. The amount of the total released ⁵¹Cr was calculated by carrying out the same procedure by using 1 mol/l hydrochloric acid solution instead of the antibody solution and human effector cell suspension and measuring amount of ⁵¹Cr in the supernatant. The cytotoxic activity (%) was calculated based on the following equation.

$$\begin{matrix}Cytotoxic \\ activity ( \% )\end{matrix} = \frac{{}^{51} Cr in sample supernatant - spontaneously released {}^{51} Cr}{total released {}^{51} Cr - spontaneously released {}^{51} Cr} \times 100$$

FIG. 23 shows a result of the measurement of ADCC activity by using effector cells of a healthy donor A at various concentrations (from 0.001 to μg/ml) of the anti-CD20 chimeric antibodies having a different ratio of antibody molecules α1,6-fucose-free sugar chain group. As shown in FIG. 23, the ADCC activity of anti-CD20 chimeric antibodies showed a tendency to increase at each antibody concentration, as the ratio of antibody molecules to which an α1,6-fucose-free sugar chain is bound increased. When the antibody concentration is low, the ADCC activity is decreased. At an antibody concentration of 0.01 μg/ml, antibodies having 26%, 35%, 44% and 96% of the α1,6-fucose-not-bound sugar chain showed almost the same high ADCC activity, but the antibody having 6% of the α1,6-fucose-not-bound sugar chain showed low ADCC activity.

4. Evaluation of ADCC Activity to CD20-Expressing Cell Line (LDH Method)

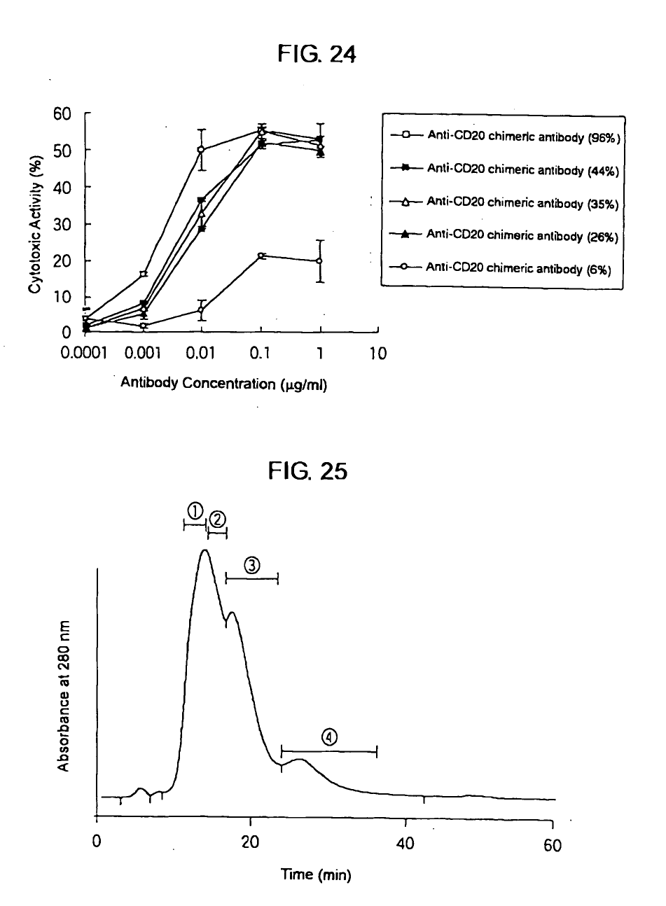

The ADCC activity to Raji cell was evaluated by the LDH (lactate dehydrogenase) activity measuring method described in item 2 of Example 2 using effector cells collected from a healthy donor B. The ratio of the effector cell to the target cell was 20:1, the final antibody concentration was 0.0001 to 1 μg/ml, the reaction was carried out at a total volume of 200 μL at 37° C. for 4 hours, and then the measurement was carried out in accordance with the item 2 of Example 2. FIG. 24 shows a result of the measurement of ADCC activity using effector cells of a healthy donor B at various concentrations (from 0.0001 to 1 μg/ml) of the anti-CD20 chimeric antibodies having a different ratio of antibody molecules to which an α1,6-fucose-free sugar chain is bound. As shown in FIG. 24, the ADCC activity of anti-CD20 chimeric antibodies showed a tendency to increase at each antibody concentration, as the ratio of antibody molecules to which an α1,6-fucose-free sugar chain is bound increased. When the antibody concentration is low, the ADCC activity is decreased. At an antibody concentration of 0.01 μg/ml, antibodies having 26%, 35%, 44% and 96% of the α1,6-fucose-not-bound sugar chain showed high ADCC activity, but the antibody having 6% of the α1,6-fucose-not-bound sugar chain showed low ADCC activity.

The results of FIG. 23 and FIG. 24 show that the ADCC activity increases in response to the ratio of antibody molecules to which an α1,6-fucose-free sugar chain is bound and that an antibody composition having about 20% or more of the ratio of antibody molecules to which an α1,6-fucose-free sugar chain is bound has sufficiently high ADCC activity, and the same results were obtained when the donor of human effector cells and the target cell was changed.

### EXAMPLE 11

Activity Evaluation of Anti-CD20 Chimeric Antibodies Having a Different Ratio of Antibody Molecules to Which a Sugar Chain Having Bisecting GlcNAc is Bound:

(1) Separation of an Anti-CD20 Chimeric Antibody by Lectin Chromatography

By using a column immobilized with lectin which has the affinity for sugar chains having bisecting GlcNAc, the anti-CD20 chimeric antibody KM3065 purified in item 3 of Example 1 was separated.

A solution containing the purified anti-CD20 chimeric antibody KM3065 was applied to a lectin column (LA-PHA-E₄, 4.6×150 mm, manufactured by Hohnen Corp.). By using LC-6A manufactured by Shimadzu as the BPLC system, the lectin chromatography was carried out at a flow rate of 0.5 ml/min and at room temperature as the column temperature. The column was equilibrated with 50 mM Tris-sulfuric acid buffer (pH 8.0), and then a solution containing the purified KM065 was injected and fluted by a linear gradient (35 minutes) of 0 M to 58 mM of potassium tetraborate (K₂B₄O₇, manufactured by Nakalai Tesque) in 50 mM Tris-sulfuric acid buffer (pH 8.0). Thereafter, the potassium tetraborate concentration was kept at 100 mM for 5 minutes, and then 50 mM Tris-sulfuric acid buffer (pH 8.0) was further passed through the column for 20 minutes to thereby separate the anti-CD20 chimeric antibody KM3065 into 4 fractions (fractions \mathring{1} to \mathring{4}) eluted during a period of 9 to 14 minutes, of 14 to 17 minutes, 17 to 22 minutes and 22 to 34 minutes (FIG. 25).

(2) Sugar Chain Analysis

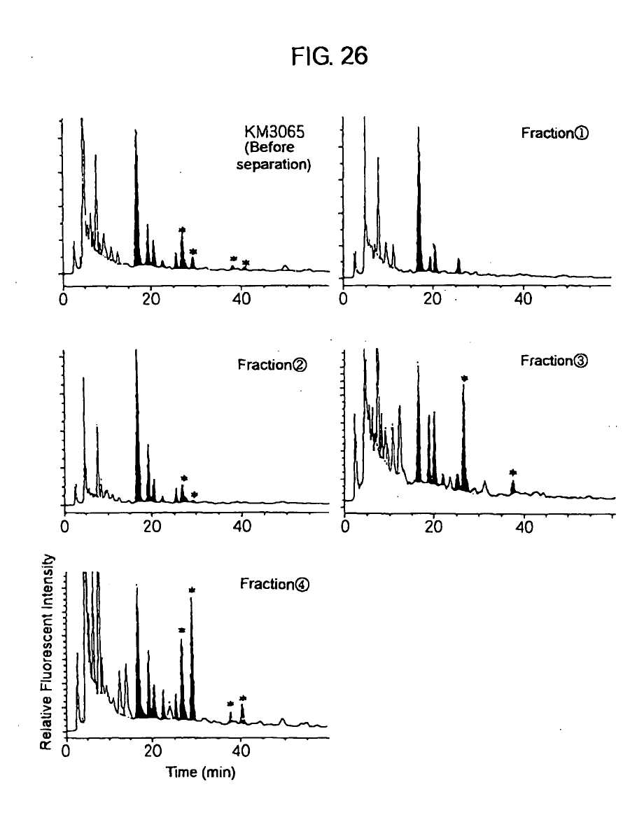

Sugar chain analysis of the thus separated 4 fractions (fractions ¢) to i) and the anti-CD20 chimeric antibody KM3065 before separation was carried out by the method described in Example 3. The PA-modified sugar chains were eluted during a period of 15 minutes to 45 minutes. When the ratio of the sugar chain having bisecting GlcNAc based on the total of peak area of each PA-modified sugar chain was calculated, the ratio of the sugar chain in the anti-CD20 chimeric antibody KM3065 before separation was 20%, whereas it was 0% in the fraction (1, 8% in the fraction \mathring{4}, 33% in the fraction \mathring{3} and 45% in the fraction \mathring{3} (FIG. 26). The ratio of the antibody molecule to which an α1,6-fucose-free sugar chain was bound was the anti-CD20 chimeric antibody KM3065 before separation: 96%, faction ( ): 93%, fraction \mathring{2}: 94%, fraction \mathring{3}: 92% and fraction \mathring{4}: 90%. Based on these results, it was confirmed that the ratio of antibody molecules to which an α1,6-fucose-free sugar chain was bound was almost uniform when calculated using a column immobilized with lectin which has the affinity for sugar chains having bisecting GlcNAc, and that anti-CD20 chimeric antibodies having different ratio of antibody molecules to which a sugar chain having bisecting GlcNAc is bound were prepared.

(3) Measurement of In Vitro Cytotoxic Activity (ADCC Activity)

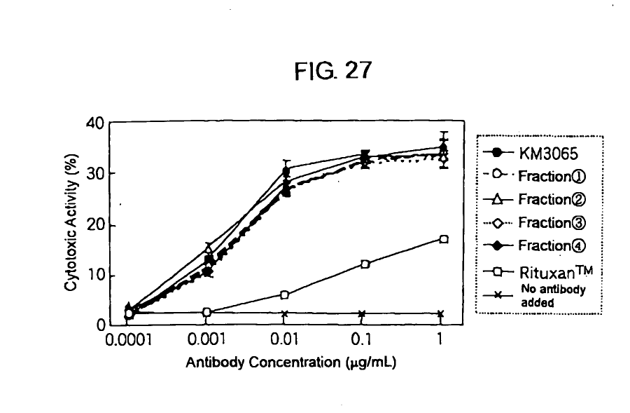

Measurement of in vitro cytotoxic activity (ADCC activity) of the 4 fractions (fractions \mathring{1} to \mathring{4}) separated by lectin chromatography and the anti-CD20 chimeric antibody KM3065 before separation was carried out by the method described in item 2 of Example 2 (FIG. 27). As a result, the 4 fractions separated by lectin chromatography showed almost the same strength of ADCC activity as that of the anti-CD20 chimeric antibody KM3065 before separation. Since antibody molecules to which an α1,6-fucose-free sugar chain is bound have almost the same ratio of 90% to 96% according to the results of the above item, it was considered that influences of the antibody molecules to which an α1,6-fucose-free sugar chain is bound on the ADCC activity are almost the same. Strength of the ADCC activity was not increased when the bisecting GlcNAc was further added to the antibody which has a high ratio of antibody molecule to which an α1,6-fucose-free sugar chain is bound and also has high ADCC activity. That is, it was found that the antibody having a high binding ratio of α1,6-fucose-free sugar chain has higher ADCC activity than the antibody having a high binding ratio of a sugar chain having α1,6-fucose, independent of the presence or absence of bisecting GlcNAc.

While the invention has been described in detail and with reference to specific embodiments thereof, it will be apparent to one skill in the art that various changes and modifications can be made therein without departing from the spirit and scope thereof. All references cited herein are incorporated in their entirety.

This application is based on Japanese application No. 2001-392753 filed on Dec. 25, 2001, No. 2002-106948 filed on Apr. 9, 2002 and No. 2002-319975 filed on Nov. 1, 2002, the entire contents of which are incorporated hereinto by reference.

What is claimed is:

1. A cell which produces an antibody composition comprising an antibody molecule which specifically binds to CD20 and has complex N-glycoside-linked sugar chains bound to the Fc region, wherein among the total complex N-glycoside-linked sugar chains bound to the Fc region in the composition, the ratio of a sugar chain in which fucose is not bound to N-acetylglucosamine in the reducing end in the sugar chain is 20% or more.

2. The cell according to claim 1, wherein the sugar chain to which fucose is not bound is a complex N-glycoside-linked sugar chain in which 1-position of fucose is not bound to 6-position of N-acetylglucosamine in the reducing end through α-bond.

3. The cell according to claim 1 or 2, wherein the activity of an enzyme relating to the synthesis of an intracellular sugar nucleotide, GDP-fucose, and/or the activity of an enzyme relating to the modification of a sugar chain in which 1-position of fucose is bound to 6-position of N-acetylglucosamine in the reducing end through α-bond in the complex N-glycoside-linked sugar chain is decreased or deleted.

4. The cell according to claim 3, wherein the enzyme relating to the synthesis of an intracellular sugar nucleotide, GDP-fucose is an enzyme selected from the group consisting of the following (a), (b) and (c):
(a) GMD (GDP-mannose 4,6-dehydratase);
(b) Fx (GDP-keto-6-deoxymannose 3,5-epimerase, 4-reductase);
(c) GFPP (GDP-beta-L-fucose pyrophosphorylase).

5. The cell according to claim 4, wherein the GMD is a protein encoded by a DNA of the following (a) or (b)
(a) a DNA comprising the nucleotide sequence represented by SEQ ID NO:41;
(b) a DNA which hybridizes with the DNA consisting of the nucleotide sequence represented by SEQ ID NO:41 under stringent conditions and encodes a protein having GMD activity.

6. The cell according to claim 4, wherein the GMD is a protein selected from the group consisting of the following (a), (b) and (c):
(a) a protein comprising the amino acid sequence represented by SEQ ID NO:61;
(b) a protein which consists of an amino acid sequence in which at least one amino acid is deleted, substituted, inserted and/or added in the amino acid sequence represented by SEQ ID NO:61 and has GMD activity;
(c) a protein which consists of an amino acid sequence having a homology of at least 80% with the amino acid sequence represented by SEQ ID NO:61 and has GMD activity.

7. The cell according to claim 4, wherein the Fx is a protein encoded by a DNA of the following (a) or (b):
(a) a DNA comprising the nucleotide sequence represented by SEQ ID NO:48;
(b) a DNA which hybridizes with the DNA consisting of the nucleotide sequence represented by SEQ. ID NO:48 under stringent conditions and encodes a protein having Fx activity.

8. The cell according to claim 4, wherein the Fx is a protein selected from the group consisting of the following (a), (b) and (c):
(a) a protein comprising the amino acid sequence represented by SEQ ID NO:62;
(b) a protein which consists of an amino acid sequence in which at least one amino acid is deleted, substituted, inserted and/or added in the amino acid sequence represented by SEQ ID NO:62 and has Fx activity;
(c) a protein which consists of an amino acid sequence having a homology of at least 80% with the amino acid sequence represented by SEQ ID NO:62 and has Fx activity.

9. The cell according to claim 4, wherein the GFPP is a protein encoded by a DNA of the following (a) or (b):
(a) a DNA comprising the nucleotide sequence represented by SEQ ID NO:51;
(b) a DNA which hybridizes with the DNA consisting of the nucleotide sequence represented by SEQ ID NO:51 under stringent conditions and encodes a protein having GFPP activity.

10. The cell according to claim 4, wherein the GFPP is a protein selected from the group consisting of the following (a), (b) and (c):
(a) a protein comprising the amino acid sequence represented by SEQ ID NO:63;
(b) a protein which consists of an amino acid sequence in which at least one amino acid is deleted, substituted, inserted and/or added in the amino acid sequence represented by SEQ ED NO:63 and has GFPP activity;
(c) a protein which consists of an amino acid sequence having a homology of at least 80% P with the amino acid sequence represented by SEQ ID NO:63 and has GFPP activity.

11. The cell according to claim 3, wherein the enzyme relating to the modification of a sugar chain in which 1-position of fucose is bound to 6-position of the N-acetylglucosamine in the reducing end through at-bond in the complex N-glycoside-linked sugar chain is α1,6-fucosyltransferase.

12. The cell according to claim 11, wherein the α1,6-fucosyltransferase is a protein encoded by a DNA of the following (a), (b), (c) and (d);
(a) a DNA comprising the nucleotide sequence represented by SEQ ID NO:1;
(b) a DNA comprising the nucleotide sequence represented by SEQ ID NO:2;
(c) a DNA which hybridizes with the DNA consisting of the nucleotide sequence represented by SEQ ID NO:1 under stringent conditions and encodes a protein having α1,6-fucosyltransferase activity;
(d) a DNA which hybridizes with the DNA consisting of the nucleotide sequence represented by SEQ ID NO:2 under stringent conditions and encodes a protein having c1,6-fucosyltransferase activity.

13. The cell according to claim 1, wherein the α1,6-fucosyltransferase is a protein selected from the group consisting of the following (a), (b), (c), (d), (e) and (f):
(a) a protein comprising the amino acid sequence represented by SEQ ID NO:23;
(b) a protein comprising the amino acid sequence represented by SEQ ID NO:24;
(c) a protein which consists of an amino acid sequence in which at least one amino acid is deleted, substituted, inserted and/or added in the amino acid sequence represented by SEQ ID NO:23 and has α1,6-fucosyltransferase activity;
(d) a protein which consists of an amino acid sequence in which at least one amino acid is deleted, substituted, inserted and/or added in the amino acid sequence represented by SEQ D NO:24 and has α1,6-fucosyltransferase activity;
(e) a protein which consists of an amino acid sequence having a homology of at least 80% with the amino acid sequence represented by SEQ ID NO:23 and has α1,6-fucosyltransferase activity;
(f) a protein which consists of an amino acid sequence having a homology of at least 80% with the amino acid sequence represented by SEQ ID NO:24 and has α1,6-fucosyltransferase activity.

14. The cell according to any one of claims 3 to 13, wherein the enzyme activity is decreased or deleted by a technique selected from the group consisting of the following (a), (b), (c), (d) and (e):
(a) a gene disruption technique targeting a gene encoding the enzyme;
(b) a technique for introducing a dominant negative mutant of a gene encoding the enzyme;
(c) a technique for introducing mutation into the enzyme;
(d) a technique for inhibiting transcription or translation of a gene encoding the enzyme,
(e) a technique for selecting a cell line resistant to a lectin which recognizes a sugar chain in which 1-position of fucose is bound to 6-position of-N-acetylglucosamine in the reducing end through α-bond in the complex N-glycoside-linked sugar chain.

15. The cell according to any one of claims 1 to 14, which is resistant to at least a lectin which recognizes a sugar chain in which 1-position of fucose is bound to 6-position of N-acetylglucosamine in the reducing end through α-bond in the complex N-glycoside-linked sugar chain.

16. The cell according to any one of claims 1 to 15, which is a cell selected from the group consisting of the following (a) to (j):
(a) a CHO cell derived from a Chinese hamster ovary tissue;
(b) a rat myeloma cell line, YB2/3HL.P2.G11.16Ag.20 cell;
(c) a mouse myeloma cell line, NS0 cell;
(d) a mouse myeloma cell line, SP2/0Ag14 cell;
(e) a BHK cell derived from a syrian hamster kidney tissue;
(f) a monkey COS cell;
(g) an antibody-producing hybridoma cell;
(h) a human leukemia cell line, Namalwa cell;
(i) an embryonic stem cell;
(j) a fertilized egg cell.

17. A transgenic non-human animal or plant or the progenies thereof into which an antibody molecule which specifically binds to CD20 and has complex N-glycoside-linked sugar chains bound to the Fc region is introduced, which produces an antibody composition comprising the antibody molecule, wherein among the total complex N-glycoside-linked sugar chains bound to the Fc region in the composition, the ratio of a sugar chain in which fucose is not bound to N-acetylglucosamine in the reducing end in the sugar chain is 20% or more.

18. The transgenic non-human animal or plant or the progenies thereof according to claim 17, wherein the sugar chain in which fucose is not bound to N-acetylglucosamine is a sugar chain in which 1-position of the fucose is not bound to 6-position of N-acetylglucosamine in the reducing end through α-bond in the N-glycoside-linked sugar chain.

19. The transgenic non-human animal or plant or the progenies thereof according to claim 17 or 18, wherein a genome is modified such that the activity of an enzyme relating to the synthesis of an intracellular sugar nucleotide, GDP-fucose and/or the activity of an enzyme relating to the modification of a sugar chain in which 1-position of fucose is bound to 6-position of N-acetylglucosamine in the reducing end through α-bond in the N-glycoside-linked sugar chain is decreased.

20. The transgenic non-human animal or plant or the progenies thereof according to claim 17 or 18, wherein a gene encoding the enzyme relating to the synthesis of an intracellular sugar nucleotide, GDP-fucose, and/or a gene encoding the enzyme relating to the modification of a sugar chain in which 1-position of fucose is bound to 6-position of N-acetylglucosamine in the reducing end through α-bond in the N-glycoside-linked sugar chain is knocked out.

21. The transgenic non-human animal or plant or the progenies thereof according to claim 19 or 20, wherein the enzyme relating to the synthesis of an intracellular sugar nucleotide, GDP-fucose is an enzyme selected from the group consisting of the following (a), (b) and (c):
(a) GMD (GDP-mannose 4,6-dehydratase);
(b) Fx (GDP-keto-6-deoxymannose 3,5-epimerase, 4-reductase);
(c) GFPP (GDP-beta-L-fucose pyrophosphorylase).

22. The transgenic non-human animal or plant or the progenies thereof according to claim 21, wherein the GMD is a protein encoded by a DNA of the following (a) or (b):
(a) a DNA comprising the nucleotide sequence represented by SEQ ID NO:41;
(b) a DNA which hybridizes with the DNA consisting of the nucleotide sequence represented by SEQ ID NO:41 under stringent conditions and encodes a protein having GMD activity.

23. The transgenic non-human animal or plant or the progenies thereof according to claim 21, wherein the Fx is a protein encoded by a DNA of the following
(a) or (b):
(a) a DNA comprising the nucleotide sequence represented by SEQ ID NO:48;
(b) a DNA which hybridizes with the DNA consisting of the nucleotide sequence represented by SEQ ID NO:48 under stringent conditions and encodes a protein having Fx activity.

24. The transgenic non-human animal or plant or the progenies thereof according to claim 21, wherein the GFPP is a protein encoded by a DNA of the following (a) or (b):
(a) a DNA comprising the nucleotide sequence represented by SEQ ID NO:51;
(b) a DNA which hybridizes with the DNA consisting of the nucleotide sequence represented by SEQ ID NO:51 under stringent conditions and encodes a protein having GFPP activity.

25. The transgenic non-human animal or plant or the progenies thereof according to claim 19 or 20, wherein the enzyme relating to the modification of a sugar chain in which 1-position of fucose is bound to 6-position of N-acetylglucosamine in the reducing end through α-bond in the N-glycoside-linked sugar chain is α1,6-fucosyltransferase.

26. The transgenic non-human animal or plant or the progenies thereof according to claim 25, wherein the α1,6-fucosyltransferase is a protein encoded by a DNA selected from the group consisting of the following (a), (b), (c) and (d)(a) a DNA comprising the nucleotide sequence represented by SEQ ID NO:1;
(b) a DNA comprising the nucleotide sequence represented by SEQ ID NO:2;
(c) a DNA which hybridizes with the DNA consisting of the nucleotide sequence represented by SEQ ID NO:1 under stringent conditions and encodes a protein having α1,6-fucosyltransferase activity;
(d) a DNA which hybridizes with the DNA consisting of the nucleotide sequence represented by SEQ ID NO:2 under stringent conditions and encodes a protein having α1,6-fucosyltransferase activity.

27. The transgenic non-human animal or plant or the progenies thereof according to any one of claims 17 to 26, wherein the transgenic non-human animal is an animal selected from the group consisting of cattle, sheep, goat, pig, horse, mouse, rat, fowl, monkey and rabbit.

28. The cell according to any one of claims 1 to 16, wherein the antibody molecule is a molecule selected from the group consisting of (a), (b), (c) and (d); (a) a human antibody;
(b) a humanized antibody;
(c) an antibody fragment comprising an Fc region of (a) or (b); (d) a fusion protein comprising an Fc region of (a) or (b).

29. The cell according to any one of claims 1 to 16 and 28, wherein the antibody molecule belongs to an IgG class.

30. The cell according to any one of claims 1 to 16, 28 and 29, wherein the antibody molecule comprises complementarity determining regions 1, 2 and 3 of an antibody light chain variable region comprising the amino acid sequences represented by SEQ ID NOs:5, 6 and 7, respectively, and/or complementarity determining regions 1, 2 and 3 of an antibody heavy chain comprising the amino acid sequences represented by SEQ ID NOs:8, 9 and 10, respectively.

31. The cell according to any one of claims 1 to 16, 28, 29 and 30, wherein the antibody molecule comprises a light chain variable region comprising the amino acid sequence represented by SEQ ID NO:12 and/or a heavy chain variable region comprising the amino acid sequence represented by SEQ ID NO:14.

32. The transgenic non-human animal or plant or the progenies thereof according to any one of claims 17 to 27, wherein the antibody molecule is a molecule selected from the group consisting of (a), (b), (c) and (d):
(a) a human antibody;
(b) a humanized antibody,
(c) an antibody fragment comprising an Fc region of (a) or (b);
(d) a fusion protein comprising an Fc region of (a) or (b).

33. The transgenic non-human animal or plant or the progenies thereof according to any one of claims 17 to 27 and 32, wherein the antibody molecule belongs to an. IgG class.

34. The transgenic non-human animal or plant or the progenies thereof according to any one of claims 17 to 27, 32 and 33, wherein the antibody molecule comprises complementarity determining regions 1, 2 and 3 of an antibody light chain variable region comprising the amino acid sequences represented by SEQ ID NOs:5, 6 and 7, respectively, and/or complementarity determining regions 1, 2 and 3 of an antibody heavy chain comprising the amino acid sequences represented by SEQ ID NOs:8, 9 and 10, respectively.

35. The transgenic non-human animal or plant or the progenies thereof according to any one of claims 17 to 27, 32, 33 and 34, wherein the antibody molecule comprises a light chain variable region comprising the amino acid sequence represented by SEQ]OD NO:12 and/or a heavy chain variable region comprising the amino acid sequence represented by SEQ ID NO:14.

36. An antibody composition which is produced by the cell according to any one of claims 1 to 16 and 28 to 31.

37. An antibody composition which is obtainable by rearing the transgenic non-human animal or plant or the progenies thereof according to any one of claims 17 to 27 and 32 to 35.

38. An antibody composition comprising an antibody molecule which specifically binds to CD20 and has complex N-glycoside-linked sugar chains bound to the Fc region, wherein among the total complex N-glycoside-linked sugar chains bound to the Fc region in the composition, the ratio of a sugar chain in which fucose is not bound to N-acetylglucosamine in the reducing end in the sugar chain is 20% or more.

39. The antibody composition according to claim 38, wherein the sugar chain to which fucose is not bound is a complex N-glycoside-linked sugar chain in which 1-position of fucose is not bound to 6-position of N-acetylglucosamine in the reducing end through α-bond.

40. The antibody composition according to claim 38, wherein the antibody molecule is a molecule selected from the group consisting of (a), (b), (c) and (d):
(a) a human antibody;
(b) a humanized antibody;
(c) an antibody fragment comprising an Fc region of (a) or (b);
(d) a fusion protein comprising an Fc region of (a) or (b).

41. The antibody composition according to any one of claims 38 to 40, wherein the antibody molecule belongs to an IgG class.

42. The antibody composition according to any one of claims 38 to 41, wherein the antibody molecule comprises complementarity determining regions 1, 2 and 3 of an antibody light chain variable region comprising the amino acid sequences represented by SEQ ID NOs:5, 6 and 7, respectively, and/or complementarity determining regions 1, 2 and 3 of an antibody heavy chain comprising the amino acid sequences represented by SEQ ID NOs:8, 9 and 10, respectively.

43. The antibody composition according to any one of claims 38 to 42, wherein the antibody molecule comprises a light chain variable region comprising the amino acid sequence represented by SEQ ID NO:12 and/or a heavy chain variable region comprising the amino acid sequence represented by SEQ ID NO:14.

44. A process for producing the antibody composition according to any one of claims 36 and 38 to 43, which comprises culturing the cell according to any one of claims 1 to 16 and 28 to 31 to form and accumulate the antibody composition in the culture; and recovering the antibody composition from the culture.

45. A process for producing the antibody composition according to any one of claims 36 and 38 to 43, which comprises rearing the transgenic non-human animal or plant or the progenies thereof according to any one of claims 17 to 27 and 32 to 35; isolating tissue or body fluid from the reared animal or plant; and recovering the antibody composition from the isolated tissue or body fluid.

46. A medicament which comprises the antibody composition according to any one of claims 36 to 43 as an active ingredient.

47. An agent for treating diseases relating to CD20, which comprises the antibody composition according to any one of claims 36 to 43 as an active ingredient.

48. The agent according to claim 47, wherein the disease relating to CD20 is a cancer or an immunological disease.
